(19) United States
(12) Patent Application Publication
(10) Pub. No.: US 2026/0007735 A1
(43) Pub. Date: Jan. 8, 2026

(54) MRNA ENCODING INFLUENZA VIRUS-LIKE PARTICLE

(71) Applicant: SANOFI PASTEUR INC., Swiftwater, PA (US)

(72) Inventors: Sophie BUFFIN, Paris (FR); Isabelle LEGASTELOIS, Paris (FR)

(21) Appl. No.: 19/237,148

(22) Filed: Jun. 13, 2025

Related U.S. Application Data
(63) Continuation of application No. PCT/EP2023/086109, filed on Dec. 15, 2023.

(30) Foreign Application Priority Data
Dec. 15, 2022 (EP) 22315327.1

(51) Int. Cl. A61K 39/145; A61K 39/00; A61P 37/04; C12N 15/86

(52) U.S. Cl. CPC A61K 39/145; A61P 37/04; C12N 15/86; A61K 2039/5258; A61K 2039/53; C12N 2760/16122; C12N 2760/16134; C12N 2760/16143; C12N 2760/16171; C12N 2760/16222; C12N 2760/16234; C12N 2760/16243; C12N 2760/16271

(57) ABSTRACT

The present invention relates to a messenger RNA (mRNA)-based immunogenic composition that is capable of inducing a mammalian cell to produce an influenza virus-like particle (VLP). The immunogenic composition comprises one or more mRNAs encoding an influenza virus matrix 1 (M1) protein and one or more influenza virus hemagglutinin (HA) proteins and/or one or more influenza virus neuraminidase (NA) proteins.

### RELATED APPLICATIONS

This application is a continuation of International Patent Application No. PCT/EP2023/086109, filed Dec. 15, 2023, which claims priority to European Patent Application No. 22315327.1, filed Dec. 15, 2022, the entire disclosures of which are hereby incorporated herein by reference.

### FIELD OF THE INVENTION

The present invention relates to a messenger RNA (mRNA)-based influenza vaccine that is capable of inducing a mammalian cell to produce a virus-like particle (VLP). As VLPs can express various clusters of epitopes on their surface, but lack viral genetic material, the vaccine is expected to elicit an immune response that is effective against multiple influenza subtypes/lineages and that is long-lasting, whilst ensuring a higher safety profile than alternative vaccination strategies.

### BACKGROUND OF THE INVENTION

Influenza viruses are enveloped, negative-stranded RNA viruses of the Orthomyxoviridae family. There are four types of influenza virus, A, B, C, and D, of which only A, B, and C are known to infect humans. A and B are the most commonly circulating types. Subtypes of influenza A are classified based on the presence of two surface glycoproteins, hemagglutinin (HA) and neuraminidase (NA). Influenza B viruses are classified by two distinct lineages, influenza B/Yamagata and influenza B/Victoria. Influenza A viruses are further subdivided into 18 HA and 11 NA subtypes. The large number of potential subtypes accounts for the high antigenic variation observed in influenza viruses.

HA and NA are considered key determinants for the establishment of a productive infection and a host immune response. HA is responsible for the initial interaction between the virus and sialic acid on the host cell receptor to promote viral entry. Following HA-mediated binding to sialic acid residues, NA acts as a biological scissor and cleaves sialic acid to facilitate viral release (Buffin et al., Vaccine. 2019: 37(46): 6857-6867; Cohen et al, Virology Journal 2013 10:321). The matrix 1 (M1) protein interacts with viral ribonucleoproteins and is tightly associated with the inner surface of the viral membrane (Peukes et al., Nature. 2020; 587: 495-498). The matrix 2 (M2) protein is a structural, transmembrane protein that forms a proton channel within the viral envelope (Pielak & Chou, Biochim Biophys Acta. 2011; 1808(2): 522-529.

The most commonly reported symptoms associated with influenza virus infection include upper respiratory tract signs of tracheobronchitis and pharyngitis together with fever, malaise, and myalgia. High-risk patient populations are more likely to present with severe disease phenotypes such as pneumonia and acute respiratory distress syndrome, which may lead to hospitalization and potentially death (Flerlage et al., Nat Rev Microbiol. 2021; 19(7): 425-441). It is estimated that over 3-5 million severe cases of influenza occur each year; hundreds of thousands of these cases result in fatality (Ali & Cowling, Annu Rev Public Health. 2021; 42: 43-57).

Vaccination remains the most successful way to prevent influenza and reduce disease burden. Currently, several vaccine approaches are employed for immunization programs, including inactivated, live attenuated, and subunit vaccines. None of the currently available influenza vaccines are particularly efficacious. This can at least partially be explained by inappropriate vaccine strain selection and the lag time between strain selection and provision of a vaccine. For instance, many current influenza vaccines are made by propagating influenza viruses in embryonic hen eggs, where the long production time causes difficulty in responding efficiently to newly emerging strains.

Thus, a need remains for the development of an influenza vaccine that can be deployed more quickly against seasonally circulating strains and is effective in preventing or reducing infection.

mRNA-based vaccines can be deployed quickly, as was demonstrated during the recent COVID-19 pandemic caused by the SARS-CoV-2 virus. Indeed, although the virus was mutating rapidly as it spread through the human population, mRNA-based vaccines remained effective in reducing the severity of infection. In addition, mRNA technology has made it possible to provide an updated vaccine in a short period of time. The updated vaccine includes an mRNA sequence encoding the SARS-CoV-2 spike glycoprotein comprising the mutations acquired by the dominant circulating virus.

An mRNA-based vaccine strategy is highly attractive for preventing influenza infections or reducing the severity of these infections.

### SUMMARY OF THE INVENTION

Virus-like particles (VLPs) have drawn the attention of vaccine researchers for decades because of their effectiveness in eliciting an immune response. However, the production of a VLP-based vaccine has remained elusive for most viruses, in particular enveloped viruses, in part due to the difficulty of manufacturing VLPs at a commercial scale.

The present disclosure demonstrates that mRNA-based vaccine technology can be used to induce the expression of influenza VLPs in a mammalian cell. Specifically, by transfecting mammalian cells with one or more messenger RNAs (mRNAs) encoding (i) an influenza virus matrix 1 (M1) protein and (ii) one or more influenza virus hemagglutinin (HA) proteins and/or one or more influenza virus neuraminidase (NA) protein, the inventors were able to induce production of VLPs in these cells that were very similar in morphology and size to a wild-type influenza virus. Using this mRNA-based approach, the inventors were able to induce mammalian cells to produce VLPs that comprised HA proteins and NA proteins originating from different influenza viruses.

Notably, at least one of the HA proteins and/or at least one of the NA proteins was/were from an influenza virus that was different from the influenza virus from which the M1 protein originated. This indicated that a single M1 protein coding sequence may be sufficient to induce the formation of VLPs with multiple HA and NA proteins from different influenza viruses on their surface.

The composition described herein can be designed to elicit an immune response that is effective against multiple influenza subtypes/lineages. As VLPs can express clusters of epitopes on their surface, but lack the complete viral genetic material, the compositions (e.g., immunogenic compositions or vaccines) described herein are expected to maintain a good safety profile while being highly immunogenic.

In particular, the invention relates to a composition (e.g., an immunogenic composition or vaccine) comprising one or more messenger RNAs (mRNAs) encoding (i) an influenza virus matrix 1 (M1) protein and (ii) one or more influenza virus hemagglutinin (HA) proteins and/or one or more influenza virus neuraminidase (NA) proteins, wherein the M1 protein and the one or more HA proteins and/or the one or more NA proteins are capable of inducing a mammalian cell to produce a virus-like particle (VLP).

In some embodiments, the VLP is between about 50 nm and about 200 nm in size. In some embodiments, the VLP is about 100 nm in size.

In some embodiments, the mammalian cell is a human cell.

In some embodiments, the one or more mRNAs encode two or more HA proteins and/or two or more NA proteins, wherein each of the two or more HA proteins and each of the two or more NA proteins are from different influenza viruses.

In some embodiments, at least one of the two or more HA proteins and/or at least one of the two or more NA proteins is/are from an influenza virus that is different from the influenza virus from which the M1 protein originates. In some embodiments, the two or more HA proteins and/or the two or more NA proteins are from different influenza A subtypes. In some embodiments, at least one of the two or more HA proteins and/or at least one of the two or more NA proteins is/are from an influenza B virus.

In some embodiments, the M1 protein is from a pandemic influenza virus (e.g., A/California/07/2009). In some embodiments, the M1 protein is from an H1N1 influenza A virus. In some embodiments, the M1 protein has a serine at position 30, an alanine at position 142, an asparagine at position 207, and a threonine at position 209. In some embodiments, the M1 protein is encoded by a separate mRNA.

In some embodiments, at least one of the two or more NA proteins has an activity of 2000 micromolar/hour (μM/h) or greater, as determined by a neuraminidase activity assay. In some embodiments, the two or more NA proteins comprise two NA proteins from different influenza A subtypes. In some embodiments, the different influenza A subtypes are N1 and N2. In some embodiments, the composition (e.g., the immunogenic composition or vaccine) is capable of inducing expression of a VLP in the mammalian cell wherein the VLP comprises the two NA proteins from different influenza A subtypes.

In some embodiments, the two or more HA proteins comprise two HA proteins from different influenza A subtypes. In some embodiments, the different influenza A subtypes are H1 and H3. In some embodiments, the composition (e.g., the immunogenic composition or vaccine) is capable of inducing expression of a VLP in the mammalian cell wherein the VLP comprises the two HA proteins from different influenza A subtypes.

In some embodiments, the one or more mRNAs are encapsulated in one or more lipid nanoparticles (LNPs).

In some embodiments, each HA protein and each NA protein are encoded by separate mRNAs. In some embodiments, the composition (e.g., the immunogenic composition or vaccine) comprises three, four, five, six, seven, eight, or nine mRNA molecules encoding the M1 protein and (i) two, three, four, five, six, seven, or eight HA proteins, (ii) two, three, four, five, six, seven, or eight NA proteins, or (iii) one, two, three, or four HA protein and one, two, three, or four NA protein. In some embodiments, the three, four, five, six, seven, eight, or nine mRNAs are encapsulated in the same LNP.

In some embodiments, wherein the composition (e.g., the immunogenic composition or vaccine) comprises one mRNA encoding the M1 protein and at least one mRNA encoding one HA protein and one NA protein. In some embodiments, the mRNAs are encapsulated in the same LNP.

In some embodiments, the composition (e.g., the immunogenic composition or vaccine) comprises a first mRNA encoding a first HA protein and a first NA protein, a second mRNA encoding a second HA protein and a second NA protein, and a third mRNA encoding the M1 protein, wherein the first HA protein and the first NA protein are from a first influenza virus, and wherein the second HA protein and the second NA protein are from a second influenza virus.

In some embodiments, the first and second influenza viruses are influenza A viruses of different subtypes. In some embodiments, the different subtypes are H1N1 and H3N2.

In some embodiments, the composition (e.g., the immunogenic composition or vaccine) further comprises a fourth mRNA encoding a third HA protein and a third NA protein, wherein the third HA protein and the third NA protein are from a third influenza virus. In some embodiments, the third influenza virus is an influenza B virus.

In some embodiments, the composition (e.g., the immunogenic composition or vaccine) further comprises a fifth mRNA encoding a fourth HA protein and a fourth NA protein, wherein the fourth HA protein and the fourth NA protein are from a fourth influenza virus. In some embodiments, the third and fourth influenza viruses are of the influenza B/Yamagata lineage and influenza B/Victoria lineage, respectively.

In some embodiments, the one, first, second, fourth and fifth mRNA, as applicable, comprise in 5′ to 3′ order (i) the coding sequence of the HA protein, (ii) a nucleotide sequence encoding an internal ribosome entry site (IRES) or a 2A peptide, and (iii) the coding sequence of the NA protein.

In some embodiments, the first, second, third, fourth and fifth mRNAs, as applicable, are encapsulated in the same LNP.

In some embodiments, the first, second and third mRNAs are encapsulated in a first LNP and the fourth and fifth mRNA are encapsulated in a second LNP. In some embodiments, the second LNP further comprises a sixth mRNA encoding an M1 protein. In some embodiments, the M1 protein encoded by the sixth mRNA is from an influenza B virus. In some embodiments, the first LNP and the second LNP comprise the same lipid components.

In some embodiments the composition (e.g., the immunogenic composition or vaccine) further comprises an mRNAs further encode an influenza virus matrix 2 (M2) protein.

In some embodiments, the one or more mRNAs are sequence-optimized.

In some embodiments, the mRNA comprises a polyadenylation (polyA) sequence comprising between about 100 nucleotides to about 500 nucleotides. In some embodiments, the polyA sequence comprises about 200 nucleotides. In some embodiments, the polyA sequence comprises about 500 nucleotides.

In some embodiments, the lipid component of the LNP(s) comprises or consists of a cationic lipid, a non-cationic lipid, a PEG-modified lipid, and optionally a sterol-based lipid.

In some embodiments, the cationic lipid is selected from cKK-E12, cKK-E10, HGT5000, HGT5001, ICE, HGT4001, HGT4002, HGT4003, TL1-01D-DMA, TL1-04D-DMA, TL1-08D-DMA, TL1-10D-DMA, OF-Deg-Lin, OF-02, GL-HEPES-E3-E12-DS-4-E10, GL-TES-SA-DMP-E18-2, GL-TES-SA-DME-E18-2, SY-3-E14-DMAPr, TL1-10D-DMA, HEP-E3-E10, HEP-E4-E10, RL3-DMA-07D, RL2-DMP-07D, cHse-E-3-E10, cHse-E-3-E12, cDD-TE-4-E12, SI-4-E14-DMAPr, TL-1-12D-DMA, SY-010, SY-011, and 4-hydroxybutyl)azanediyl)bis(hexane-6,1-diyl)bis(2-hexyldecanoate (ALC-0315).

In some embodiments, the non-cationic lipid selected from DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DEPE 1,2-dierucoyl-sn-glycero-3-phosphoethanolamine, DOPC (1,2-dioleyl-sn-glycero-3-phosphotidylcholine), DPPE (1,2-dipalmitoyl-sn-glycero-3-phosphoethanolamine), DMPE (1,2-dimyristoyl-sn-glycero-3-phosphoethanolamine), and DOPG (1,2-dioleoyl-sn-glycero-3-phospho-(1′-rac-glycerol)).

In some embodiments, the PEG-modified lipid is selected from DMG-PEG-2K and 2-[(polyethylene glycol)-2000]-N,N-ditetradecylacetamide (ALC-0159).

In some embodiments, the sterol-based lipid is cholesterol.

In some embodiments, the cationic lipid is selected from cKK-E10, OF-02, GL-HEPES-E3-E12-DS-4-E10, and ALC-0315. In some embodiments, the PEG-modified lipid is selected from DMG-PEG2K or ALC-0159. In some embodiments, the non-cationic lipid is selected from DOPE or DSPC.

In some embodiments, the LNP(s) is/are between about 70 nm and about 150 nm in size.

The invention also relates to a pharmaceutical composition that comprise a composition (e.g., an immunogenic composition) disclosed herein and one or more pharmaceutically acceptable excipients. In some embodiments, the one or more pharmaceutically acceptable excipients is selected from a salt, a sugar, a buffering reagent and combinations thereof. In some embodiments, the salt is sodium chloride, potassium chloride or a combination of both. In some embodiments, the sugar is a disaccharide. In some embodiments, the disaccharide is sucrose or trehalose. In some embodiments, the buffering reagent is selected from phosphate, Tris, imidazole and histidine. In some embodiments, the buffering reagent is phosphate or Tris.

The invention also relates to a method of eliciting an immune response against one or more influenza viruses in a subject, wherein the method comprises administering a composition (e.g., an immunogenic composition, vaccine, or a pharmaceutical composition) disclosed herein to the subject. In some embodiments, the immune response is effective in reducing the severity of one or more symptoms of an infection with the one or more influenza viruses in the subject. In some embodiments, the immune response is effective in preventing an infection with the one or more influenza viruses in the subject.

In some embodiments, the subject is human. In some embodiments, the subject is pregnant. In some embodiments, the subject is 65 years or older. In some embodiments, the subject is 70 years or older.

### BRIEF DESCRIPTION OF THE DRAWINGS

Embodiments of the invention will be described, by way of example, with reference to the following drawings, in which:

FIG. 1 illustrates NA activity in the supernatant of HEK293T cells transfected with a single mRNA encoding an NA protein of subtype N1 or N2, or with multiple mRNAs encoding HA, NA and M1 proteins, as indicated in the bar graph. Enzymatic activity of NA is reported as M/h and mean values (N=3) are indicated above the bars. No NA activity was detected in the supernatant of mock-transfected HEK293T cells (no mRNA). PBS served as a negative control.

FIG. 2 illustrates protein expression of an H3-subtype HA protein in the lysate of HEK293T cells transfected with an mRNA encoding the H3-subtype HA protein (H3) and one or more additional mRNA encoding an H1-subtype HA protein (H1), an M1 protein (M1), or one or more NA proteins (N1, N2), as indicated. The lysates were separated on a gel. A Western blot was performed with a polyclonal anti-H3 antibody. Mock-transfected cells (no mRNA) served as a negative control (NC). Purified VLPs obtained from HEK293T cells transiently transfected with expression plasmids encoding the H1, N1, H3, N2, and M1 proteins served as a positive control (PC). The lanes of the gel were loaded from left to right as follows: Molecular weight marker (M); H3; H3/M1; H3/N2/M1, H1N1/H3N2/M1; H1/H3/M1; negative control (NC); positive control (PC); molecular weight marker (M).

FIG. 3 shows representative cryogenic transmission electron microscopy (Cryo-TEM) images of VLPs from the supernatant of mock and mRNA-transfected HEK293T cells. The transfected cell supernatant was concentrated to Amicon® Ultra Centrifugal Filters with pore size at 100 kDa to remove cell debris and small proteins. Concentrated VLPs were kept at −80 C.° for further examinations. Panel A shows mock-transfected cells (no mRNA). No VLPs were visible in this condition. In contrast, panel B illustrates the presence of spherical vesicles on the surface of cells transfected with separate mRNAs encoding the influenza virus HA, NA and M1 proteins (H3/N2/M1). Similarly, panel C illustrates the presence of spherical vesicles on the surface of cells transfected with separate mRNAs encoding the HA and NA proteins of two different influenza viruses (H1, H3, N1 and N2, respectively) and an mRNA encoding the M1 protein. The VLPs have a size of approximately 100 nm, similar to the size of influenza viruses. Notably, the expression of HA and NA proteins from different influenza viruses did not interfere with VLP formation.

FIG. 4 illustrates NA activity in the supernatant of Expi293F cells transfected with an mRNA encoding an NA protein, an mRNA encoding an HA protein, and an mRNA encoding an M1 protein. The NA and HA proteins were matched and derived from Darwin (type A), Wisconsin (type A), Phuket (type B) and Austria (type B) influenza strains, respectively. The M1 protein was from a strain different to that from which the HA and NA protein sequences were derived (see Example 6 for further details). Enzymatic activity of NA is reported as M/h. No NA activity was detected in the supernatant of mock-transfected Expi293F cells (lipofectant only, negative control). Purified recombinant VLPs served as a positive control.

FIG. 5 illustrates expression of influenza type A (panel A) and type B (panel B) HA proteins in the lysates and supernatant (SN; concentrated and non-concentrated) of Expi293F cells transfected with an mRNA encoding an NA protein, an mRNA encoding an HA protein, and an mRNA encoding an M1 protein. The NA and HA proteins were matched and derived from Darwin (type A), Wisconsin (type A), Phuket (type B) and Austria (type B) strains, respectively. The M1 protein was from a strain different to that from which the HA and NA protein sequences were derived (see Example 6 for further details). After separation on a gel, a Western blot was performed with a monoclonal anti-HA antibody directed towards type A (panel A) or type B (panel B). Mock-transfected cells (lipofectant only) served as a negative control. Recombinant influenza type B HA (rHA_B) served as a positive control. A molecular weight marker (MW) was also included in a separate lane. Bands corresponding to full-length HA and the HA2 subunit are framed by dashed lines.

FIG. 6 illustrates expression of M1 protein in the lysates and concentrated supernatants (panel A) and non-concentrated supernatants (panel B) of Expi293F cells transfected with an mRNA encoding an NA protein, an mRNA encoding an HA protein, and an mRNA encoding an M1 protein. The NA and HA proteins were matched and derived from Darwin (type A), Wisconsin (type A), Phuket (type B) and Austria (type B) strains, respectively. The M1 protein was from a strain different to that from which the HA and NA protein sequences were derived (see Example 6 for further details). After separation on a gel, a Western blot was performed with a polyclonal anti-M1 antibody. Mock-transfected cells (lipofectant only) served as a negative control. Purified VLPs served as a positive control. A molecular weight marker (MW) was also included in a separate lane. M1 was detected in the cell lysates and concentrated supernatant of transfected cells (panel A) but not the non-concentrated supernatant samples (panel B).

FIG. 7 shows representative cryogenic transmission electron microscopy (Cryo-TEM) images from the concentrated supernatants of mock and mRNA-transfected Expi293F cells. Panel A shows mock-transfected cells (lipofectant alone). No VLPs were visible in this condition. Panels B-E show representative images of supernatants of Expi293F cells transfected with an mRNA encoding an NA protein, an mRNA encoding an HA protein, and an mRNA encoding an M1 protein. The NA and HA proteins were matched and derived from the influenza strains Darwin (type A; panel B), Wisconsin (type A; panel C), Phuket (type B; panel D) and Austria (type B; panel E), respectively, as indicated above each panel. The M1 protein was from a strain different to that from which the HA and NA protein sequences were derived (see Example 6 for further details). Only heterogeneous smooth vesicles (white arrows), likely artefacts from the transfection process, and protein debris (black outlined arrows) were observed in supernatants of mock-transfected cells (see illustrative images in panel A). VLPs were detected in each of the supernatants of cells transfected with the mRNAs (see panels B-E; filled black arrows). VLPs could be clearly distinguished from protein debris (black outlined arrows; see panels B-D) and smooth vesicles (white arrows; see panel E). The VLPs had a size of approximately 50-150 nm.

### DEFINITIONS

In order for the present invention to be more readily understood, certain terms are first defined below. Additional definitions for the following terms and other terms are set forth throughout the specification.

As used in this specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

Exemplary methods and materials are described below, although methods and materials similar or equivalent to those described herein can also be used in the practice or testing of the present invention. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein.

As used herein, the term “mRNA” refers to a polyribonucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, in vitro transcribed, or chemically synthesized. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. A typical mRNA comprises a 5′ cap, a 5′ untranslated region (5′ UTR), a protein-coding region, a 3′ untranslated region (3′ UTR), and a 3′ tail. In some embodiments, the tail structure is a poly(C) tail. More typically, the tail structure is a poly(A) tail.

As used herein the term “sequence-optimized” is used to describe a nucleotide sequence that is modified relative to a naturally-occurring or wild-type nucleic acid. Such modifications may include, e.g., codon optimization as well as the use of 5′ UTRs and 3′ UTRs which are not normally associated with the naturally-occurring or wild-type nucleic acid. As used herein, the terms “codon optimization” and “codon-optimized” refer to modifications of the codon composition of a naturally-occurring or wild-type nucleic acid encoding a peptide, polypeptide or protein that do not alter its amino acid sequence, thereby improving protein expression of said nucleic acid. In the context of the present invention, “codon optimization” may also refer to the process by which one or more optimized nucleotide sequences are arrived at by removing with filters less than optimal nucleotide sequences from a list of nucleotide sequences, such as filtering by guanine-cytosine content, codon adaptation index, presence of destabilizing nucleic acid sequences or motifs, and/or presence of pause sites and/or terminator signals.

As used herein, the term “template DNA” (or “DNA template”) relates to a DNA molecule comprising a nucleic acid sequence encoding an mRNA transcript to be synthesized by in vitro transcription (IVT). The template DNA is used as template for IVT in order to produce the mRNA transcript encoded by the template DNA. The template DNA comprises all elements necessary for IVT, particularly a promoter element for binding of a DNA-dependent RNA polymerase, such as, e.g., T3, T7 and SP6 RNA polymerases, which is operably linked to the DNA sequence encoding a desired mRNA transcript. Furthermore the template DNA may comprise primer binding sites 5′ and/or 3′ of the DNA sequence encoding the mRNA transcript to determine the identity of the DNA sequence encoding the mRNA transcript, e.g., by PCR or DNA sequencing. The “template DNA” in the context of the present invention may be a linear or a circular DNA molecule. As used herein, the term “template DNA” may refer to a DNA vector, such as a plasmid DNA, which comprises a nucleic acid sequence encoding the desired mRNA transcript.

As used herein, the term “subject” refers to a mammal, such as a human or other animal. Typically, a subject is a human. The subject can be male or female and can be any suitable age, including infant, juvenile, adolescent, adult, and geriatric subjects.

As used herein, the term “vaccine composition” or “vaccine” refers to a composition that is capable of generating a protective immune response in a subject. As used herein, a “protective immune response” refers to an immune response that protects a subject from infection (prevents infection or prevents the development of disease associated with infection) or reduces the symptoms of infection (for instance an infection by an influenza virus). Vaccines may elicit both prophylactic (preventative) and therapeutic responses.

All technical and scientific terms used herein have the same meaning as commonly understood by one of ordinary skill in the art to which this application belongs and as commonly used in the art to which this application belongs. The publications and other reference materials referenced herein to describe the background of the invention and to provide additional detail regarding its practice are hereby incorporated by reference.

### DETAILED DESCRIPTION OF THE INVENTION

The present invention provides a composition (e.g., an immunogenic composition or vaccine) comprising one or more mRNAs that encode (i) an influenza virus matrix 1 (M1) protein and (ii) one or more influenza virus haemagglutinin (HA) proteins and/or one or more influenza virus neuraminidase (NA) proteins, wherein the M1 protein and the one or more HA proteins and/or the one or more NA proteins are capable of inducing a mammalian cell to produce a virus-like particle (VLP).

### Virus-Like Particles

Virus-like particles (VLPs) are structures that assemble upon expression of viral proteins and can mimic native virion structure. They have a similar structure and shape to native viruses but lack the viral genome. VLPs of enveloped viruses typically include lipid membranes that derive from the cell in which they were expressed. One or more viral glycoproteins are typically incorporated into the lipid membrane and act as target antigens that can be recognized by immune cells to produce neutralizing antibodies.

The budding of influenza VLPs from cells is dependent on the expression of HA and NA and, in particular, the cytoplasmic tails of HA and NA. The efficient release of VLPs from the cell surface requires the presence of HA and sialidase activity provided by NA. Additionally, the effective production of VLPs in mammalian cells, particularly human cells, is dependent on the presence of M1 protein. VLPs produced in the presence of M1 protein are very similar to complete VLPs.

Accordingly, in a particular embodiment, the present invention provides a composition (e.g., an immunogenic composition or vaccine) comprising one or more mRNAs that encode (i) an influenza virus matrix 1 (M1) protein and (ii) one or more influenza virus haemagglutinin (HA) proteins, and (iii) one or more influenza virus neuraminidase (NA) proteins. Such compositions are particularly effective in inducing mammalian, and particularly human, cells to produce virus-like particles (VLPs). VLP production can be visualized using electron microscopy (EM) methods, as described herein, e.g., in Examples 4 and 5. For example, the one or more mRNAs that encode the M1 protein, the one or more HA proteins, and the one or more NA proteins can be used to transfect HEK293 cells or other mammalian cells, and VLP production can be observed using cryogenic EM methods as described herein.

In some embodiments, the VLP is between about 50 nm and about 200 nm in size. In a specific embodiment, the VLP is about 100 nm in size.

### M1 Protein

The matrix 1 (M1) protein is a 252-residue structural protein that forms a coat underneath the lipid bilayer of the viral particles. As a consequence, it does not experience the same evolutionary pressure as the surface proteins, haemagglutinin (HA) and neuraminidase (NA). Indeed, the M1 protein is one of the slowest-evolving proteins encoded by the influenza virus genome. It is encoded by the M gene of the influenza virus genome. The M gene was found to evolve 5- to 10-fold more slowly than the HA gene.

M1 protein includes both MHC class-I and MHC class-II T-cell epitopes. A particularly common MHC class-II T-cell epitope overlaps with a nuclear transport sequence.

The high conservation of its amino acid sequence coupled with the presence of T-cell epitopes suggest that inclusion of an mRNA encoding the M1 protein may be effective in eliciting an immune response against multiple strains of influenza virus.

In some embodiments, the M1 protein is from a pandemic influenza virus. In some embodiments, the M1 protein is from an H1N1 influenza A virus (e.g., A/California/07/2009 or A/Puerto Rico/8/1934). In some embodiments, the M1 protein comprises M1 protein comprises the amino acid serine (S) at position 30, the amino acid alanine (A) at position 142, the amino acid asparagine (N) at position 207 and the amino acid threonine (T) at position 209. In some embodiments, the M1 protein is capable of forming VLPs comprising NA and HA protein from any influenza A strain. In some embodiments, the M1 protein is capable of forming VLPs comprising NA and HA protein from any influenza B strain.

When transfecting mammalian cells with one or more mRNAs encoding an HA protein, one or more mRNAs encoding an NA protein and an mRNA encoding an M1 protein derived from an H1N1 influenza A virus (A/California/07/2009), the inventors observed efficient budding of VLPs at the plasma membrane, even when the HA and NA proteins were derived from an influenza virus other than A/California/07/2009.

### Haemagglutinin

The hemagglutinin (HA) protein is an integral membrane protein that is associated with the viral envelope. HA is the major antigen of the influenza virus, and outnumbers NA by five- to ten-fold on the virion surface. There are 18 known HA subtypes subdivided into group 1 (H1, H2, H5, H6, H8, H9, H11, H12, H13, H16, H17, and H18) and group 2 (H3, H4, H7, H10, H14, and H15).

In some embodiments, a composition of the invention (e.g., an immunogenic composition or a vaccine of the invention) comprises at least one mRNA encoding at least one HA protein of subtype H1, H2, H3, H4, H5, H6, H7, H8, H9, H10, H11, H12, H13, H14, H15, H16, H17, or H18. Common seasonal influenza A subtypes include H1 and H3. Thus, in some embodiments, the at least one mRNA encodes at least one HA protein of subtype H1 or H3. Influenza A strains with pandemic potential may include an H5, H7, H9 or H10 subtype. Accordingly, in some embodiments, the at least one mRNA encodes at least one HA protein of subtype H5, H7, H9 or H10.

More typically, a composition of the invention (e.g., an immunogenic composition or a vaccine of the invention) comprises more than one (e.g., two or more) mRNAs encoding HA proteins, wherein each HA protein is selected from a different subtype. The HA proteins may be selected from any of subtypes H1, H2, H3, H4, H5, H6, H7, H8, H9, H10, H11, H12, H13, H14, H15, H16, H17, and H18. More commonly, the more than one mRNAs encode at least one HA protein of each of groups 1 and 2. For example, the more than one mRNAs may encode an HA protein of subtype H1 and an HA protein of subtype H3.

In some embodiments, a composition of the invention (e.g., the immunogenic composition or vaccine of the invention) comprises at least one mRNA encoding at least one HA protein of influenza B virus. In some embodiments, the influenza B virus is of the Yamagata lineage. In some embodiments, the influenza B virus is of the Victoria lineage. In some embodiments, the composition of the invention (e.g., the immunogenic composition or vaccine of the invention) comprises one or more mRNA(s) encoding an HA protein of the influenza B/Yamagata lineage and an HA protein of the influenza B/Victoria lineage.

### Neuraminidase

The neuraminidase (NA) protein is another integral membrane protein associated with the viral envelope. NA assembles as a tetramer of four identical polypeptides which fold into distinct structural domains comprising the cytoplasmic tail, the transmembrane region, the stalk, and the catalytic head. High sequence conservation of the N-terminus region of the cytoplasmic tail has been observed. There are 11 known subtypes of NA (N1-N11). NA is responsible for the removal of sialic acids from cellular receptors and newly synthesized HA and NA on nascent virions. This enzymatic activity prevents virus aggregation, avoids viral binding onto the dying host cell and promotes viral release and infection of new cell targets.

In some embodiments, a composition of the invention (e.g., an immunogenic composition or vaccine of the invention) comprises at least one mRNA encoding at least one NA protein of subtype N1, N2, N3, N4, N5, N6, N7, N8, N9, N10, or N11. For example, common seasonal influenza A subtypes include N1 and N2. Thus, in some embodiments, the at least one mRNA encodes at least one NA protein of subtype N1 or N3. Influenza A strains with pandemic potential may include an N1, N2, N3, N9 or N7. Accordingly, in some embodiments, the at least one mRNA encodes at least one NA protein of subtype N1, N2, N3, N9 or N7.

More typically, a composition of the invention (e.g., an immunogenic composition or vaccine of the invention) comprises more than one (e.g., two or more) mRNAs encoding NA proteins, wherein each NA protein is selected from a different subtype. The NA proteins may be selected from any of subtypes N1, N2, N3, N4, N5, N6, N7, N8, N9, N10, and N11. More commonly, the more than one mRNAs encode at least one NA protein of the N1 subtype and at least one NA protein of the N2 subtype.

In some embodiments, the composition of the invention (e.g., the immunogenic composition or vaccine of the invention) comprises at least one mRNA encoding at least one NA protein of influenza B virus. In some embodiments, the influenza B virus is of the Yamagata lineage. In some embodiments, the influenza B virus is of the Victoria lineage. In some embodiments, the composition of the invention (e.g., the immunogenic composition or vaccine of the invention) comprises one or more mRNA(s) encoding an NA protein of the influenza B/Yamagata lineage and an NA protein of the influenza B/Victoria lineage.

The inventors have observed that the NA proteins of influenza A subtype N2 strains can be 2.5- to 3-fold more active than NA proteins of influenza A subtype N1. Accordingly, it may be desirable to include an mRNA encoding an NA protein of high activity of 2000 M/hour or greater in combination with an mRNA encoding an NA protein of lower activity (e.g., less than 1000 M/hour) to ensure efficient budding of the VLPs from mRNA-transfected cells. Accordingly, in some embodiments, a composition of the invention (e.g., an immunogenic composition or vaccine of the invention) comprises at least one mRNA encoding an NA protein of high activity (e.g., NA protein of the N2 subtype) and at least one mRNA encoding an NA protein of low activity (e.g., an NA protein of the N1 subtype). Enzymatic activity of the NA protein can be determined as described herein, e.g., in Example 2. Specifically, a fluorescence-based assay can be used to determine the activity of the NA protein that employs 4-methylumbelliferone-N-acetyl neuraminic acid (MUNANA) as a substrate.

### M2 Protein

Coexpression of an HA protein and an NA protein with the influenza virus matrix 2 (M2) protein may further enhance the release of these proteins from a host cell.

### VLP-Forming Compositions

A VLP-forming composition of the invention (e.g., an immunogenic composition or vaccine of the invention) typically comprises multiple mRNAs encoding an M1 protein, one or more HA proteins and one or more NA proteins. The one or more HA proteins and the one or more NA proteins may be encoded by the same mRNA or by separate mRNAs. In some embodiments, each HA protein and each NA protein are encoded by separate mRNAs.

Typically, the M1 protein is encoded by a separate mRNA. In some embodiments, the M1 protein is from an influenza virus that is distinct from the influenza virus from which at least one HA protein and/or at least on NA protein are derived that are encoded by the one or more other mRNAs in the composition (e.g., the immunogenic composition or vaccine). In some embodiments, the M1 protein is from an H1N1 influenza A virus (e.g., A/California/07/2009) and/or comprises the amino acid serine (S) at position 30, the amino acid alanine (A) at position 142, the amino acid asparagine (N) at position 207 and the amino acid threonine (T) at position 209.

In some embodiments, a composition of the invention (e.g., an immunogenic composition or vaccine of the invention) comprises two, three, four, five, six, seven, eight, nine, or more mRNA molecules encoding a M1 protein and (i) one or more HA proteins, (ii) one or more NA proteins, or (iii) a combination of one or more HA proteins and NA proteins.

In some embodiments, a composition of the invention (e.g., an immunogenic composition or vaccine of the invention) comprises three, four, five, six, seven, eight, nine, or more mRNA molecules encoding an M1 protein and (i) two or more HA proteins, (ii) two or more NA proteins, or (iii) at least one HA protein and at least one NA protein.

In some embodiments, the HA and NA proteins are from an influenza A virus. In some embodiments, the one or more HA proteins comprise an H1 subtype and the one or more NA proteins comprise an N1 subtype. In some embodiments, the one or more HA proteins comprise an H2 subtype and the one or more NA proteins comprise an N2 subtype. In some embodiments, the one or more HA proteins comprise an H5 subtype and the one or more NA proteins comprise an N1 subtype. In some embodiments, the one or more HA proteins comprise an H3 subtype and the one or more NA proteins comprise an N2 subtype. In some embodiments, the one or more HA proteins comprise an H7 subtype and the one or more NA proteins comprise an N3, N7 or N9 subtype. In some embodiments, the one or more HA proteins comprise an H9 subtype and the one or more NA proteins comprise an N2 subtype. In some embodiments, the one or more HA proteins comprise an H10 subtype and the one or more NA proteins comprise an N7 subtype.

In some embodiments, the one or more HA proteins comprise subtypes H1 and H3 and the one or more NA proteins comprise subtypes N1 and N2. In some embodiments, the one or more HA proteins comprise an HA protein of the influenza B/Yamagata lineage or influenza B/Victoria lineage. In some embodiments, the one or more NA proteins comprise an NA protein of the influenza B/Yamagata lineage or influenza B/Victoria lineage. In some embodiments, the one or more HA proteins comprise HA proteins of the subtypes H1 and H3 and one or both HA protein(s) of the influenza B/Yamagata lineage or influenza B/Victoria lineage and the one or more NA proteins comprise HA proteins of the subtypes N1 and N2 and one or both NA protein(s) of the influenza B/Yamagata lineage or influenza B/Victoria lineage.

In one embodiment, a composition of the invention (e.g., an immunogenic composition or vaccine of the invention) comprises one mRNA encoding an H3 HA protein, one mRNA molecule encoding an H1 HA protein, one mRNA encoding an HA protein of the influenza B/Yamagata lineage, one mRNA encoding an HA protein of the influenza B/Victoria lineage, and one mRNA encoding an M1 protein.

In one embodiment, a composition of the invention (e.g., an immunogenic composition or vaccine of the invention) comprises one mRNA encoding an H3 HA protein, one mRNA encoding an N2 NA protein, one mRNA encoding an H1 HA protein, one mRNA encoding an N1 NA protein, one mRNA encoding an HA protein from the influenza B/Yamagata lineage, one mRNA encoding an NA protein from the influenza B/Yamagata lineage, one mRNA encoding an HA protein from the influenza B/Victoria lineage, one mRNA encoding an NA protein from the influenza B/Victoria lineage, and one mRNA encoding an M1 protein.

In some embodiments, a composition of the invention (e.g., an immunogenic composition or vaccine of the invention) comprises a first mRNA encoding a first HA protein and a first NA protein, a second mRNA encoding a second HA protein and a second NA protein, and a third mRNA encoding an M1 protein. Typically, the first HA protein and the first NA protein are from a first influenza virus (e.g., H1 and N1 from influenza A), and the second HA protein and the second NA protein are from a second influenza virus (e.g., H3 and N2 from influenza A).

In some embodiments, the first influenza virus is an H1N1 influenza A virus and the second influenza virus is an H3N2 influenza A virus. In some embodiments, the first influenza virus is an influenza A virus (e.g., H1N1 or H3N2) and the second influenza virus is an influenza virus B virus (e.g., influenza B/Yamagata or influenza B/Victoria).

In some embodiments, a composition of the invention (e.g., an immunogenic composition or a vaccine of the invention) comprises a fourth mRNA encoding a third HA protein and a third NA protein from a third influenza virus. In some embodiments, the first influenza virus is an H1N1 influenza A virus, the second influenza virus is an H3N2 influenza virus and the third influenza virus is an influenza virus B virus (e.g., influenza B/Yamagata or influenza B/Victoria).

In some embodiments, a composition of the invention (e.g., an immunogenic composition or vaccine of the invention) comprises a fourth mRNA encoding a third HA protein and a third NA protein from a third influenza virus and a fifth mRNA encoding a fourth HA protein and a fourth NA protein from a fourth influenza virus. In some embodiments, the first influenza virus is an H1N1 influenza A virus, the second influenza virus is an H3N2 influenza A virus, the third influenza virus is an influenza virus B virus of the Yamagata lineage and the fourth influenza virus is of the Victoria lineage.

In some embodiments, the bicistronic first, second, fourth and fifth mRNAs comprise an internal ribosome entry site (IRES) between the two coding sequences encoding the HA and NA proteins. An IRES functions by acting as a second ribosome recruitment site, allowing translation initiation to occur additionally at an internal region of the mRNA.

In some embodiments, the IRES is derived from a virus. In some embodiments, the virus is a ribovirus. In some embodiments, the ribovirus is a picornavirus, hepacivirus, pestivirus, hepatitis virus, flavivirus, or retrovirus. In some embodiments, the ribovirus is a picornavirus. In some embodiments, the picornavirus is dicistrovirus or encephalomyocarditis virus (EMCV).

In some embodiments, the bicistronic first, second, fourth and fifth mRNAs comprise a nucleotide sequence encoding a self-cleaving peptide between the two coding sequences encoding the HA and NA proteins. In some embodiments, the self-cleaving peptide is a 2A peptide. In some embodiments, the self-cleaving peptide is a 2A peptide. The 2A peptide typically comprises approximately 18-25 amino acids. In some embodiments, the 2A peptide is derived from a virus. In some embodiments, the 2A peptide is P2A, T2A, E2A or F2A.

During translation of the upstream coding sequence, the 2A peptide makes the ribosome skip the synthesis of the glycine and proline peptide bond at the C-terminal end of the 2A peptide, causing separation between the 2A peptide and the protein encoded by the downstream coding sequence. As a result, the upstream protein comprises residues derived from the 2A peptide at its C terminus. The protein encoded by the downstream coding sequence comprises a proline at its N terminus, also derived from the 2A peptide.

The coding sequences of the HA protein and the NA protein in a bicistronic mRNA may be arranged in either order (HA followed by NA, or NA followed by HA). As the HA protein typically outnumbers NA by five- to ten-fold on the surface of native virus particles, it may be advantageous to position HA first (i.e., at the 5′ position of the polycistronic mRNA) such that the HA protein is expressed at higher levels than the NA protein.

For example, the coding sequence downstream of the IRES is typically expressed at much lower levels (typically 10-20%) as compared to the upstream coding sequence in the bicistron. Accordingly, a bicistronic mRNA comprising the coding sequence for the HA protein, the IRES and the coding sequence of the NA protein in 5′ to 3′ order may result in the formation of VLPs that comprise the HA protein and the NA protein at a ratio similar to that of a native virus particle.

Similarly, the coding sequence downstream of the nucleotide sequence encoding the self-cleaving peptide is typically expressed at lower levels as compared to the upstream coding sequence in the bicistron. For example, the translation product N-terminal of a 2A peptide routinely accumulates in 2- to 5-fold molar excess over that C-terminal of a 2A peptide. Accordingly, a bicistronic mRNA comprising the coding sequence for the HA protein, a nucleotide sequence encoding a self-cleaving peptide (e.g., a 2A peptide) and the coding sequence of the NA protein in 5′ to 3′ order may likewise result in the formation of VLPs that comprise the HA protein and the NA protein at a ratio similar to that of a native virus particle.

In some embodiments, a composition of the invention (e.g., an immunogenic composition or vaccine of the invention) comprises a first mRNA encoding at least a first HA protein and a second HA protein, a second mRNA encoding at least a first NA protein and a second NA protein, and a third mRNA encoding an M1 protein. Typically, the first HA protein and the second HA protein (and any further HA proteins) are from different subtypes (e.g., H1 and H3 from influenza A) and/or lineages (e.g., Victoria and Yamagata), and the first NA protein and the second NA protein (and any further NA proteins) are from different subtypes (e.g., N1 and N2 from influenza A) and/or lineages.

In some embodiments, the coding sequences for the first and second HA proteins (and any further HA proteins) and/or the coding sequences for the first and second NA proteins (and any further NA proteins) are separated by nucleotide sequence(s) encoding a self-cleaving peptide. In some embodiments, the self-cleaving peptide is a 2A peptide. The 2A peptide typically comprises about 18-25 amino acids. In some embodiments, the 2A peptide is derived from a virus. In some embodiments, the 2A peptide is P2A, T2A, E2A or F2A.

In some embodiments, the first mRNA encoding the first and second HA proteins (and any further HA proteins) and the second mRNA encoding the first and second NA proteins (and any further NA proteins) comprise the same 5′ UTR sequence. In some embodiments, the first mRNA encoding the first and second HA proteins and the second mRNA encoding the first and second NA proteins comprise different 5′ UTR sequences. For example, using different 5′ UTR sequences may be advantageous to achieve an HA to NA ratio similar to that found in native virus particles.

Coexpression of one or more HA proteins and one or more NA proteins with an M2 protein may further enhance the release of VLPs from cells. Accordingly, a composition according to the invention (e.g., an immunogenic composition or vaccine according to the invention) may further comprise an mRNA encoding a matrix 2 (M2) protein. In the influenza virus genome, the M1 protein and the M2 protein are encoded in different but partially overlapping reading frames. Thus, in some embodiments, the M2 protein is encoded by the same mRNA as the M1 protein, e.g., replicating the arrangement of the coding sequences for the M1 and M2 proteins in wild-type influenza virus genome. Alternatively, the M2 protein may be encoded by a separate mRNA.

### Strain Selection

Influenza viruses are constantly evolving and therefore frequent updates of the virus strains included in a seasonal influenza vaccine are necessary. The World Health Organization (WHO) monitors circulating respiratory viruses, such as influenza, and analyzes surveillance data to provide an annual recommendation for influenza vaccine compositions. In some embodiments, the one or more HA and NA proteins are from influenza virus strains recommended by the WHO in their annual recommendation for influenza vaccine compositions.

In certain embodiments, at least one of the one or more influenza virus proteins comprises an influenza virus HA protein and/or an influenza virus NA protein having a molecular sequence identified or designed from a machine learning model and in certain embodiments, at least one of the one or more ribonucleic acid molecules encode one or more influenza virus proteins having a molecular sequence identified or designed from a machine learning model.

In an embodiment, the composition comprises further comprise one or more mRNA molecules encoding a machine learning influenza virus HA having a molecular sequence identified or designed from a machine learning model, wherein the one or more machine learning influenza virus HA may be selected from an H1 HA, an H3 HA, an HA from an influenza B/Victoria lineage, an HA from an influenza B/Yamagata lineage, or a combination thereof.

When selecting one or more machine learning influenza virus HAs, any machine learning algorithm may be used. For example, envisioned herein are any of the machine learning algorithms and methods disclosed in PCT Application Nos. WO 2021/080990 A1, entitled Systems and Methods for Designing Vaccines, and WO 2021/080999 A1, entitled Systems and Methods for Predicting Biological Responses, both of which are incorporated by reference in their entireties herein.

mRNAs
Structural Elements of mRNAs

A typical mRNA in accordance with the invention comprises a 5′ cap, a 5′ untranslated region (5′ UTR), a protein-coding region, a 3′ untranslated region (3′ UTR), and a 3′ tail. The presence of the cap is important in providing resistance to nucleases found in most eukaryotic cells. The presence of a “tail” serves to protect the mRNA from exonuclease degradation. In some embodiments, a 5′ cap and/or a 3′ tail may be added after mRNA synthesis. In other embodiments, the 5′ cap and/or a 3′ tail sequences are included in the DNA template sequences used in in vitro transcription (IVT) reaction.

### 5′ Cap

In a specific embodiment, the mRNA of the invention comprises a 5′ cap with the following structure:

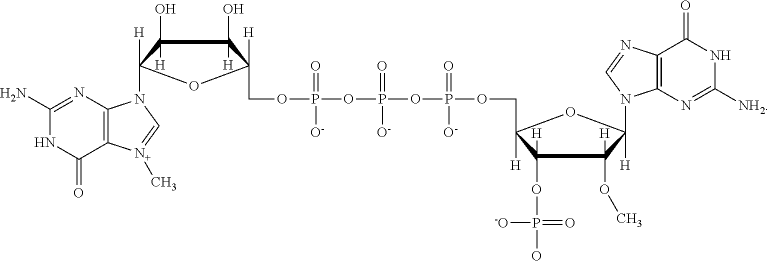

A 5′ cap may be added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G. Additional cap structures are described in published U.S. Application No. US 2016/0032356 and published U.S. Application No. US 2018/0125989, which are incorporated herein by reference.

### 3′ Tail

In a typical embodiment, the tail structure of the mRNA comprises a poly(A) tail. In some embodiments, the tail structure of the mRNA comprises a poly(C) tail. In some embodiments, the tail structure comprises at least 50 adenosine or cytosine nucleotides. In a typical embodiment, the tail structure is approximately 100-500 nucleotides in length. For example, a tail structure (e.g., a poly(A) tail) of 100-250 nucleotides in length may be particularly useful in therapeutic uses of mRNA.

A poly(A) or poly(C) tail on the 3′ terminus of mRNA typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, respectively. In some embodiments, a tail structure includes combination of poly(A) and poly(C) tails with various lengths described herein. In some embodiments, the mRNA comprises a polyA sequence comprising between 100 nucleotides to about 500 nucleotides. In one embodiment, the mRNA comprises a polyA sequence of about 200 nucleotides. In another embodiment, the mRNA comprises a polyA sequence of about 500 nucleotides.

In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

### 5′ UTRs and 3′ UTRs

In some embodiments, the mRNA disclosed herein may comprise a 5′ or 3′ UTR that is derived from a gene distinct from the one encoded by the mRNA transcript (i.e., the UTR is a heterologous UTR).

In certain embodiments, the 5′ and/or 3′ UTR sequences can be from mRNAs which are stable (e.g., globin, actin, GAPDH, tubulin, histone, or citric acid cycle enzymes) to increase the stability of the mRNA. For example, a 5′ UTR sequence may include a partial sequence of a CMV immediate-early 1 (IE1) gene, or a fragment thereof, to improve the nuclease resistance and/or improve the half-life of the mRNA. Also contemplated is the inclusion of a sequence encoding human growth hormone (hGH), or a fragment thereof, to the 3′ end or untranslated region of the mRNA. Generally, these modifications improve the stability and/or pharmacokinetic properties (e.g., half-life) of the mRNA relative to their unmodified counterparts, and include, for example, modifications made to improve such mRNA resistance to in vivo nuclease digestion.

Exemplary 5′ UTRs include a sequence from a CMV immediate-early 1 (IE1) gene (U.S. Publication Nos. 2014/0206753 and 2015/0157565, each of which is incorporated herein by reference), or the sequence of SEQ ID NO: 4 disclosed in U.S. Publication No. 2016/0151409, which is incorporated herein by reference.

In various embodiments, the 5′ UTR may be from the 5′ UTR of a TOP gene. TOP genes are typically characterized by the presence of a 5′-terminal oligopyrimidine (TOP) tract. Furthermore, most TOP genes are characterized by growth-associated translational regulation. However, TOP genes with a tissue specific translational regulation are also known. In certain embodiments, the 5′ UTR from the 5′ UTR of a TOP gene lacks the 5′ TOP motif (the oligopyrimidine tract) (e.g., U.S. Publication Nos. 2017/0029847, 2016/0304883, 2016/0235864, and 2016/0166710, each of which is incorporated herein by reference).

In certain embodiments, the 5′ UTR is from a ribosomal protein Large 32 (L32) gene (U.S. Publication No. 2017/0029847, supra).

In certain embodiments, the 5′ UTR is from the 5′ UTR of an hydroxysteroid (17-b) dehydrogenase 4 gene (HSD17B4) (U.S. Publication No. 2016/0166710, supra).

In certain embodiments, the 5′ UTR is from the 5′ UTR of an ATP5A1 gene (U.S. Publication No. 2016/0166710, supra).

In some embodiments, an internal ribosome entry site (IRES) is used instead of a 5′ UTR.

5′ UTR and 3′UTR sequences suitable for use with the invention are described in WO2012/075040, which is incorporated herein by reference. In some embodiments, the 5′UTR comprises a nucleic acid sequence set forth in SEQ ID NO: 1 of WO2012/075040. In some embodiments, the 3′UTR comprises a nucleic acid sequence set forth in SEQ ID NO:3 of WO2012/075040.

Typically, from 5′ to 3′, the mRNA construct comprises a 5′ cap as described in paragraph [0079], a 5′ UTR as described in paragraph [0121], one or more coding sequence(s) in accordance with the invention, a 3′ UTR as described in paragraph [0121], and a poly(A) tail of 100-500 nucleotides.

### Nucleotides

In some embodiments, the mRNA comprises naturally-occurring nucleosides (or unmodified nucleosides; i.e., adenosine, guanosine, cytidine, and uridine). In some embodiments, the mRNA comprises one or more modified nucleosides, such as nucleoside analogs (e.g., adenosine analog, guanosine analog, cytidine analog, or uridine analog). The presence of one or more nucleoside analogs may render an mRNA more stable and/or less immunogenic than a control mRNA with the same sequence, but containing only naturally-occurring nucleosides.

In some embodiments, the mRNA comprises both unmodified and modified nucleosides. In some embodiments, the one or more modified nucleosides is a nucleoside analog. In some embodiments, the one or more modified nucleosides comprises at least one modification selected from a modified sugar, and a modified nucleobase. In some embodiments, the mRNA comprises one or more modified internucleoside linkages.

In some embodiments, the modified nucleoside comprises at least one modification selected from a modified sugar, and a modified nucleobase relative to the corresponding naturally occurring ribonucleotide.

The modified nucleoside can be a modified uridine, cytidine, adenine, or guanine. Some exemplary chemical modifications of nucleosides in the mRNA molecule include, e.g., pyridine-4-one ribonucleoside, 5-aza-uridine, 2-thio-5-aza-uridine, 2-thiouridine, 4-thio pseudouridine, 2-thio-pseudouridine, 5-hydroxyuridine, 3-methyluridine, 5-carboxymethyl uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N⁶-methyladenosine, N⁶-isopentenyladenosine, N⁶-(cis-hydroxyisopentenyl) adenosine, 2-methylthio-N⁶-(cis-hydroxyisopentenyl) adenosine, N⁶-glycinylcarbamoyladenosine, N⁶-threonylcarbamoyladenosine, 2-methylthio-N⁶-threonyl carbamoyladenosine, N⁶,N⁶-dimethyladenosine, 7-methyladenine, 2-methylthioadenine, 2-methoxyadenine, inosine, 1-methyl-inosine, wyosine, wybutosine, 7-deaza guanosine, 7-deaza-8-aza-guanosine, 6-thio guanosine, 6-thio-7-deazaguanosine, 6-thio-7-deaza-8-aza-guanosine, 7-methyl-guanosine, 6-thio-7-methylguanosine, 7-methylinosine, 6-methoxy guanosine, 1-methylguanosine, N²-methylguanosine, N²,N²-dimethylguanosine, 8-oxo-guanosine, 7-methyl-8-oxo-guanosine, 1-methyl-6-thio-guanosine, N²-methyl-6-thio-guanosine, and N²,N²-dimethyl-6-thio-guanosine.

In some embodiments, the modified nucleoside in the mRNA molecule is a modified uridine selected from pseudouridine, pyridine-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy uridine, 5-aminoallyl-uridine, 5-halo-uridine (e.g., 5-iodom uridine or 5-bromo uridine), 3-methyl uridine, 5-methoxy-uridine, uridine-5-oxyacetic acid, uridine-5-oxyacetic acid methyl ester, 5-carboxymethyl-uridine, 1-carboxymethyl-pseudouridine, 5-carboxyhydroxymethyl uridine, 5-carboxyhydroxymethyl-uridine methyl ester, 5-methoxycarbonylmethyluridine, 5-methoxycarbonylmethyl-2-thio-uridine, 5-aminomethyl-2-thiouridine, 5-methylaminomethyl uridine, 5-methylaminomethyl-2-thio-uridine, 5-methylaminomethyl-2-selenouridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl pseudouridine, 5-taurinomethyl-2-thio-uridine, 1-taurinomethyl-4-thio-pseudouridine, 5-methyl-uridine (m⁵U, e.g., having the nucleobase deoxythymine), 1-methyl-pseudouridine, 5-methyl-2-thio-uridine, 1-methyl-4-thio-pseudouridine, 4-thio-1-methyl-pseudouridine, 3-methyl-pseudouridine), 2-thio-1-methyl-pseudouridine, 1-methyl-1-deaza-pseudouridine, 2-thio-1-methyl-1-deaza-pseudouridine, dihydrouridine, dihydropseudouridine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine, 1-methyl-3-(3-amino-3-carboxypropyl) pseudouridine, 5-(isopentenylaminomethyl) uridine, 5-(isopentenylaminomethyl)-2-thio-uridine, alpha-thio-uridine, 2′-O-methyl uridine, 5,2′-O-dimethyl uridine, 2′-O-methyl-pseudouridine, 2-thio-2′-O-methyl uridine, 5-methoxycarbonylmethyl-2′-O-methyl uridine, 5-carbamoylmethyl-2′-O-methyl uridine, 5-carboxymethylaminomethyl-2′-O-methyl uridine, 3,2′-O-dimethyl uridine, 5-(isopentenylaminomethyl)-2′-O-methyl uridine, 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino) uridine.

In some embodiments, the modified uridine is selected from N1-methylpseudouridine, pseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine

In some embodiments, the modified nucleoside is a modified cytosine selected from 5-aza-cytidine, 6-aza-cytidine, pseudoisocytidine, 3-methylcytidine, N⁴-acetyl cytidine, 5-formyl-cytidine, N⁴-methylcytidine, 5-methylcytidine, 5-halo cytidine (e.g., 5-iodo cytidine), 5-hydroxy methylcytidine, 1-methyl-pseudoisocytidine, pyrrolo-cytidine, pyrrolo-pseudoisocytidine, 2-thio-cytidine, 2-thio-5-methylcytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio zebularine, 2-thio-zebularine, 2-methoxy cytidine, 2-methoxy-5-methylcytidine, 4-methoxy pseudoisocytidine, 4-methoxy-1-methyl-pseudoisocytidine, lysidine, alpha-thio-cytidine, 2′-O-methylcytidine, 5,2′-O-dimethyl cytidine, N⁴-acetyl-2′-O-methylcytidine, N⁴,2′-O-dimethyl cytidine, 5-formyl-2′-O-methylcytidine, N⁴,N⁴,2′-O-trimethyl cytidine, 1-thio-cytidine, 2′-F-ara-cytidine, 2′-F cytidine, and 2′-OH-ara-cytidine.

In some embodiments, the modified nucleoside is a modified pyrimidine nucleoside. In some embodiments, the modified ribonucleoside is selected from pseudouridine, N1-methylpseudouridine, 5-methylcytidine, 5-methoxyuridine, and any combination thereof. In some embodiments, both cytosine and uracil are replaced with modified nucleosides (e.g., N1-methylpseudouridine and 5-methylcytidine).

In some embodiments, the modified nucleoside is a modified adenine selected from 2-amino purine, 2,6-diamino purine, 2-amino-6-halo purine (e.g., 2-amino-6-chloro purine), 6-halo purine (e.g., 6-chloro purine), 2-amino-6-methyl purine, 8-azido adenosine, 7-deaza-adenine, 7-deaza-8-aza adenine, 7-deaza-2-amino purine, 7-deaza-8-aza-2-amino purine, 7-deaza-2,6-diamino purine, 7-deaza-8-aza-2,6-diamino purine, 1-methyladenosine, 2-methyl adenine, N⁶-methyladenosine, 2-methylthio-N⁶-methyladenosine, N⁶-isopentenyl adenosine, 2-methylthio-N⁶-isopentenyl adenosine, N⁶-(cis-hydroxyisopentenyl) adenosine, 2-methylthio-N⁶-(cis-hydroxyisopentenyl)adenosine, N⁶-glycinylcarbamoyl adenosine, N⁶-threonylcarbamoyl adenosine, N⁶-methyl-N⁶-threonylcarbamoyl adenosine, 2-methylthio-N⁶-threonylcarbamoyl adenosine, N⁶,N⁶-dimethyl adenosine, N⁶-hydroxynorvalylcarbamoyl adenosine, 2-methylthio-N⁶-hydroxynorvalylcarbamoyl adenosine, N⁶-acetyl adenosine, 7-methyladenine, 2-methylthio-adenine, 2-methoxyadenine, alpha-thio-adenosine, 2′-O-methyladenosine, N⁶,2′-O-dimethyl adenosine, N⁶,N⁶,2′-O-trimethyl adenosine, 1,2′-O-dimethyl adenosine, 2′-O-ribosyl adenosine (phosphate), 2-amino-N⁶-methyl purine, 1-thio-adenosine, 8-azido-adenosine, 2′-F-ara-adenosine, 2′-F adenosine, 2′-OH-ara-adenosine, and N⁶-(19-amino-pentaoxanonadecyl) adenosine.

In some embodiments, the modified nucleoside is a modified guanine selected from inosine, 1-methyl inosine, wyosine, methylwyosine, 4-demethyl wyosine, isowyosine, wybutosine, peroxywybutosine, hydroxywybutosine, undermodified hydroxywybutosine, 7-deaza-guanosine, queuosine, epoxyqueuosine, galactosyl queuosine, mannosyl queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methylguanosine, 6-thio-7-methylguanosine, 7-methyl inosine, 6-methoxy guanosine, 1-methylguanosine, N²-methyl-guanosine, N²,N²-dimethyl guanosine, N²′7-dimethyl guanosine, N²,N²-7-dimethyl guanosine, 8-oxo-guanosine, 7-methyl-8-oxo-guanosine, 1-methylguanosine, N²-methyl-6-thio-guanosine, N²,N²-dimethyl-6-thio-guanosine, alpha-thio-guanosine, 2′-O-methylguanosine, N²-methyl-2′-O-methylguanosine, N²,N²-dimethyl-2′-O-methylguanosine, 1-methyl-2′-O-methylguanosine, N²-7-dimethyl-2′-O-methylguanosine, 2′-O-methyl inosine, 1,2′-O-dimethyl inosine, 2′-O-ribosyl guanosine (phosphate), 1-thio-guanosine, O⁶-methylguanosine, 2′-F-ara guanosine, and 2′-F guanosine.

In some embodiments, the modified nucleoside is a nucleoside analogue selected from 2-aminoadenosine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, pseudouridine (e.g., N1-methylpseudouridine), 2-thiouridine, and 2-thiocytidine. See, e.g., U.S. Pat. No. 8,278,036 or WO 2011/012316 for a discussion of 5-methylcytidine, pseudouridine, and 2-thio-uridine and their incorporation into mRNA.

In some embodiments, the modified nucleoside is selected from pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, and 2′-O-methyl uridine.

In an mRNA for use with the invention, a modified nucleotide typically takes the place of a naturally occurring nucleotide. Accordingly, the one or more mRNAs of the invention may comprise both unmodified and modified nucleotides. Such mRNAs can be prepared by including a modified nucleotide in the IVT reaction mixture, typically in place of a naturally occurring nucleotide (e.g., N1-methylpseudouridine in place of uridine). This results in mRNA in which 100% of the naturally occurring nucleotide is replaced by a corresponding modified nucleotide (e.g., 100% of the uridines are replaced with N1-methyl-pseudouridine). In some embodiments, only a portion of the naturally occurring nucleoside (e.g., at least 1%, 5%, 10%, 15%, 20% or 25% of the naturally occurring nucleoside) is replaced with a modified nucleoside. In some embodiments, one or more naturally occurring nucleosides is replaced with a modified ribonucleoside. For example, two or more ribonucleosides may be modified ribonucleosides (e.g., uridines may be replaced with 2-thio-uridine and cytidines may be replaced with 5-methylcytidine). For example, 25% of the uridines may be replaced with 2-thio-uridine and/or and 25% of cytidine residues may be replaced with 5-methylcytidine.

### Generation of Optimized Nucleotide Sequences

In some embodiments, the one or more mRNAs for use with the present invention are sequence-optimized. For example, the coding sequences of the one or more mRNA may be modified relative to their naturally occurring counterparts to (a) improve the yield of full-length mRNAs during in vitro synthesis, and (b) to maximize expression of the encoded protein after delivery of the mRNA to a target cell in vivo. Sequence motifs that favor rapid degradation of the mRNA in the target cell have also been removed.

A process for generating optimized nucleotide sequences may first include generating a list of codon-optimized sequences and then applying three filters to the list. Specifically, it applies a motif screen filter, guanine-cytosine (GC) content analysis filter, and codon adaptation index (CAI) analysis filter to produce an updated list of optimized nucleotide sequences. The updated list no longer includes nucleotide sequences containing features that are expected to interfere with effective transcription and/or translation of the encoded polypeptide.

### Codon Optimization

The genetic code has 64 possible codons. Each codon comprises a sequence of three nucleotides. The usage frequency for each codon in the protein-coding regions of the genome can be calculated by determining the number of instances that a specific codon appears within the protein-coding regions of the genome, and subsequently dividing the obtained value by the total number of codons that encode the same amino acid within protein-coding regions of the genome.

A codon usage table contains experimentally derived data regarding how often, for the particular biological source from which the table has been generated, each codon is used to encode a certain amino acid. This information is expressed, for each codon, as a percentage (0 to 100%), or fraction (0 to 1), of how often that codon is used to encode a certain amino acid relative to the total number of times a codon encodes that amino acid.

Codon usage tables are stored in publicly available databases, such as the Codon Usage Database (Nakamura et al. (2000) Nucleic Acids Research 28(1), 292; available online at https://www.kazusa.or.jp/codon/), and the High-performance Integrated Virtual Environment-Codon Usage Tables (HIVE-CUTs) database (Athey et al., (2017), BMC Bioinformatics 18(1), 391; available online at http://hive.biochemistry.gwu.edu/review/codon).

During the first step of codon optimization, codons are removed from a first codon usage table which reflects the frequency of each codon in a given organism (e.g., a mammal or human) if they are associated with a codon usage frequency which is less than a threshold frequency (e.g., 10%). The codon usage frequencies of the codons not removed in the first step are normalized to generate a normalized codon usage table. An optimized nucleotide sequence encoding an amino acid sequence of interest is generated by selecting a codon for each amino acid in the amino acid sequence based on the usage frequency of the one or more codons associated with a given amino acid in the normalized codon usage table. The probability of selecting a certain codon for a given amino acid is equal to the usage frequency associated with the codon associated with this amino acid in the normalized codon usage table.

The codon-optimized sequences of the invention are generated by a computer-implemented method for generating an optimized nucleotide sequence. The method comprises: (i) receiving an amino acid sequence, wherein the amino acid sequence encodes a peptide, polypeptide, or protein; (ii) receiving a first codon usage table, wherein the first codon usage table comprises a list of amino acids, wherein each amino acid in the table is associated with at least one codon and each codon is associated with a usage frequency; (iii) removing from the codon usage table any codons associated with a usage frequency which is less than a threshold frequency; (iv) generating a normalized codon usage table by normalizing the usage frequencies of the codons not removed in step (iii); and (v) generating an optimized nucleotide sequence encoding the amino acid sequence by selecting a codon for each amino acid in the amino acid sequence based on the usage frequency of the one or more codons associated with the amino acid in the normalized codon usage table. The threshold frequency can be in the range of 5%-30%, in particular 5%, 10%, 15%, 20%, 25%, or 30%. In the context of the present invention, the threshold frequency is typically 10%.

The step of generating a normalized codon usage table comprises: (a) distributing the usage frequency of each codon associated with a first amino acid and removed in step (iii) to the remaining codons associated with the first amino acid; and (b) repeating step (a) for each amino acid to produce a normalized codon usage table. In some embodiments, the usage frequency of the removed codons is distributed equally amongst the remaining codons. In some embodiments, the usage frequency of the removed codons is distributed amongst the remaining codons proportionally based on the usage frequency of each remaining codon. “Distributed” in this context may be defined as taking the combined magnitude of the usage frequencies of removed codons associated with a certain amino acid and apportioning some of this combined frequency to each of the remaining codons encoding the certain amino acid.

The step of selecting a codon for each amino acid comprises: (a) identifying, in the normalized codon usage table, the one or more codons associated with a first amino acid of the amino acid sequence; (b) selecting a codon associated with the first amino acid, wherein the probability of selecting a certain codon is equal to the usage frequency associated with the codon associated with the first amino acid in the normalized codon usage table; and (c) repeating steps (a) and (b) until a codon has been selected for each amino acid in the amino acid sequence.

The step of generating an optimized nucleotide sequence by selecting a codon for each amino acid in the amino acid sequence (step (v) in the above method) is performed n times to generate a list of optimized nucleotide sequences.

### Motif Screen

A motif screen filter is applied to the list of optimized nucleotide sequences. Optimized nucleotide sequences encoding any known negative cis-regulatory elements and negative repeat elements are removed from the list to generate an updated list.

For each optimized nucleotide sequence in the list, it is also determined whether it contains a termination signal. Any nucleotide sequence that contains one or more termination signals is removed from the list generating an updated list. In some embodiments, the termination signal has the following nucleotide sequence: 5′-X₁ATCTX₂TX₃-3′, wherein X₁, X₂ and X₃ are independently selected from A, C, T or G. In some embodiments, the termination signal has one of the following nucleotide sequences: TATCTGTT; and/or TTTTTT; and/or AAGCTT; and/or GAAGAGC; and/or TCTAGA. In a typical embodiment, the termination signal has the following nucleotide sequence: 5′-X₁AUCUX₂UX₃-3′, wherein X₁, X₂ and X₃ are independently selected from A, C, U or G. In a specific embodiment, the termination signal has one of the following nucleotide sequences: UAUCUGUU; and/or UUUUUU; and/or AAGCUU; and/or GAAGAGC; and/or UCUAGA.

### Guanine-Cytosine (GC) Content

The method further comprises determining a guanine-cytosine (GC) content of each of the optimized nucleotide sequences in the updated list of optimized nucleotide sequences. The GC content of a sequence is the percentage of bases in the nucleotide sequence that are guanine or cytosine. The list of optimized nucleotide sequences is further updated by removing any nucleotide sequence from the list, if its GC content falls outside a predetermined GC content range.

Determining a GC content of each of the optimized nucleotide sequences comprises, for each nucleotide sequence: determining a GC content of one or more additional portions of the nucleotide sequence, wherein the additional portions are non-overlapping with each other and with the first portion, and wherein updating the list of optimized sequences comprises: removing the nucleotide sequence if the GC content of any portion falls outside the predetermined GC content range, optionally wherein determining the GC content of the nucleotide sequence is halted when the GC content of any portion is determined to be outside the predetermined GC content range. In some embodiments, the first portion and/or the one or more additional portions of the nucleotide sequence comprise a predetermined number of nucleotides, optionally wherein the predetermined number of nucleotides is in the range of: 5 to 300 nucleotides, or 10 to 200 nucleotides, or 15 to 100 nucleotides, or 20 to 50 nucleotides. In the context of the present invention, the predetermined number of nucleotides is typically 30 nucleotides. The predetermined GC content range can be 15%-75%, or 40%-60%, or, 30%-70%. In the context of the present invention, the predetermined GC content range is typically 30%-70%.

A suitable GC content filter in the context of the invention may first analyze the first 30 nucleotides of the optimized nucleotide sequence, i.e., nucleotides 1 to 30 of the optimized nucleotide sequence. Analysis may comprise determining the number of nucleotides in the portion with are either G or C, and determining the GC content of the portion may comprise dividing the number of G or C nucleotides in the portion by the total number of nucleotides in the portion. The result of this analysis will provide a value describing the proportion of nucleotides in the portion that are G or C, and may be a percentage, for example 50%, or a decimal, for example 0.5. If the GC content of the first portion falls outside a predetermined GC content range, the optimized nucleotide sequence may be removed from the list of optimized nucleotide sequences.

If the GC content of the first portion falls inside the predetermined GC content range, the GC content filter may then analyze a second portion of the optimized nucleotide sequence. In this example, this may be the second 30 nucleotides, i.e., nucleotides 31 to 60, of the optimized nucleotide sequence. The portion analysis may be repeated for each portion until either: a portion is found having a GC content falling outside the predetermined GC content range, in which case the optimized nucleotide sequence may be removed from the list, or the whole optimized nucleotide sequence has been analyzed and no such portion has been found, in which case the GC content filter retains the optimized nucleotide sequence in the list and may move on to the next optimized nucleotide sequence in the list.

### Codon Adaptation Index (CAI)

The method further comprises determining a codon adaptation index (CAI) of each of the optimized nucleotide sequences in the most recently updated list of optimized nucleotide sequences. The CAI of a sequence is a measure of codon usage bias and can be a value between 0 and 1. The most recently updated list of optimized nucleotide sequences is further updated by removing any nucleotide sequence if its CAI is less than or equal to a predetermined codon adaptation index threshold. The CAI threshold can 0.7, or 0.75, or 0.8, or 0.85, or 0.9. The inventors have found that optimized nucleotide sequences with a CAI equal to or greater than 0.8 deliver very high protein yield. Therefore, in the context of the invention, the CAI threshold is typically 0.8.

A codon adaptation index (CAI) may be calculated, for each optimized nucleotide sequence, in any way that would be apparent to a person skilled in the art, for example as described in “The codon adaptation index—a measure of directional synonymous codon usage bias, and its potential applications” (Sharp and Li, 1987. Nucleic Acids Research 15(3), p. 1281-1295); available online at:
https://www.ncbi.nlm.nih.gov/pmc/articles/PMC340524/.

Implementing a codon adaptation index (CAI) calculation may include a method according to, or similar to, the following. For each amino acid in a sequence, a weight of each codon in a sequence may be represented by a parameter termed relative adaptiveness (w₁). Relative adaptiveness may be computed from a reference sequence set, as the ratio between the observed frequency of the codon fᵢ and the frequency of the most frequent synonymous codon fj for that amino acid. The CAI of a sequence may then be calculated as the geometric mean of the weight associated to each codon over the length of the sequence (measured in codons). The reference sequence set used to calculate CAI may be the same reference sequence set from which a codon usage table used with methods of the invention is derived.

### In Vitro Transcription

mRNAs of the invention may be synthesized according to any of a variety of known methods. Various methods are described in published U.S. Application No. US 2018/0258423 and international patent publication WO 2018/157153 (incorporated herein by reference), and can be used to practice the present invention. For example, mRNAs according to the present invention may be synthesized via in vitro transcription (IVT). Briefly, IVT is typically performed with a linear or circular DNA template or DNA vector containing a promoter, a pool of ribonucleotide triphosphates, a buffer system that may include DTT and magnesium ions, and an appropriate RNA polymerase (e.g., T3, T7, or SP6 RNA polymerase), DNase I, pyrophosphatase, and/or RNase inhibitor. The exact conditions will vary according to the specific application.

For the preparation of mRNA by IVT, a DNA template or DNA vector may be transcribed in vitro. A suitable DNA template or DNA vector typically has a promoter, for example a T3, T7 or SP6 promoter, for in vitro transcription (IVT), followed by desired nucleotide sequence for desired mRNA and a termination signal (terminator).

In one aspect, the invention provides a DNA vector encoding an mRNA comprising an optimized nucleotide sequence described herein. In some embodiments, the DNA vector further comprises a promoter and/or a terminator. In one embodiment, the promoter is a SP6 RNA polymerase promoter. In another embodiment, the promoter is a T7 RNA polymerase promoter.

### Post-Synthesis Purification

Various methods may be used to purify mRNA after synthesis. In some embodiments, the mRNA is purified using Tangential Flow Filtration (TFF). Suitable purification methods include those described in published U.S. Application No. US 2016/0040154, published U.S. Application No. US 2015/0376220, published U.S. Application No. US 2018/0251755, published U.S. Application No. US 2018/0251754, U.S. Provisional Application No. 62/757,612 filed on Nov. 8, 2018, and U.S. Provisional Application No. 62/891,781 filed on Aug. 26, 2019, all of which are incorporated by reference herein and may be used to practice the present invention. It is advantageous to purify the mRNA of the invention which may be included in pharmaceutical compositions in some embodiments of the invention, as the purity requirements for mRNA products are more stringent for therapeutic applications.

In some embodiments, the mRNA is purified before capping and tailing. In some embodiments, the mRNA is purified after capping and tailing. In some embodiments, the mRNA is purified both before and after capping and tailing. In some embodiments, the mRNA is purified either before or after or both before and after capping and tailing, by centrifugation. In some embodiments, the mRNA is purified either before or after or both before and after capping and tailing, by filtration. In some embodiments, the mRNA is purified either before or after or both before and after capping and tailing, by TFF.

### Lipid Nanoparticles (LNPs)

The invention also provides a lipid nanoparticle (LNP) encapsulating the one or more mRNAs for use with the invention. Typically, a lipid nanoparticle suitable for use with the present invention comprises one or more cationic lipids, one or more non-cationic lipids (e.g., DOPE and/or cholesterol), and one or more PEG-modified lipids (e.g., DMG-PEG2K).

A typical lipid nanoparticle for use with the invention is composed of four lipid components: a cationic lipid (e.g., cKKE10, OF-02 or ALC-0315), a non-cationic lipid (e.g., DOPE or DSPC), a cholesterol-based lipid (e.g., cholesterol) and a PEG-modified lipid (e.g., DMG-PEG-2K or 2-[(polyethylene glycol)-2000]-N,N-ditetradecylacetamide (ALC-0159)). The molar ratio of cationic lipid to non-cationic lipid to cholesterol to PEG-modified lipid typically is between about 30-60:25-35:20-30:1-15, respectively. An exemplary LNP in accordance with the invention may be composed of a cationic lipid selected from cKK-E10, OF-02 and ALC-0315; a non-cationic lipid selected from DOPE and DSPC; a cholesterol-based lipid such as cholesterol; and a PEG-modified lipid such as DMG-PEG-2K and ALC-0159.

In some embodiments, a lipid nanoparticle comprises no more than three distinct lipid components. An exemplary lipid nanoparticle is composed of three lipid components: a cationic lipid (e.g., a sterol-based cationic lipid), a non-cationic lipid (e.g., DOPE or DEPE) and a PEG-modified lipid (e.g., DMG-PEG2K). In a specific embodiment, the three distinct lipid components are HGT4002, DOPE and DMG-PEG2K. In an exemplary embodiment, HGT4002, DOPE and DMG-PEG2K are present in a molar ratio of approximately 60:35:5, respectively. Such LNPs may be particularly suitable for aerosol delivery of the mRNAs of the invention.

The lipid nanoparticles for use in the invention can be prepared by various techniques which are presently known in the art. Such methods are described, e.g., in published U.S. Application No. US 2011/0244026, published U.S. Application No. US 2016/0038432, published U.S. Application No. US 2018/0153822, published U.S. Application No. US 2018/0125989 and U.S. Provisional Application No. 62/877,597, filed Jul. 23, 2019, all of which are incorporated herein by reference.

### Cationic Lipids

A cationic lipid affords a positively charged environment at low pH to facilitate efficient encapsulation of the negatively charged mRNA drug substance. Various cationic lipids which are suitable for use in LNPs are known in the art. These include, for example, DOTAP (1,2-dioleyl-3-trimethylammonium propane), DODAP (1,2-dioleyl-3-dimethylammonium propane), DOTMA (N-[1-(2,3-dioleyloxy)propyl]-N,N,N-trimethylammonium chloride), DLinKC2DMA, DLin-KC2-DM, and C12-200. Exemplary cationic lipids suitable for use in the LNPs, compositions, pharmaceutical compositions and methods of the invention are described herein and include, for instance, the cationic lipids as described in International Patent Publication WO 2010/144740, which is incorporated herein by reference. In some embodiments, the LNPs, compositions, pharmaceutical compositions and methods of the present invention include a cationic lipid, (6Z,9Z,28Z,31Z)-heptatriaconta-6,9,28,31-tetraen-19-yl 4-(dimethylamino) butanoate, having a compound structure of;

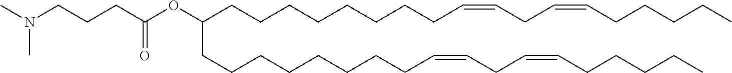 [SMILES: CCCCC/C=C\C/C=C\CCCCCCCCC(CCCCCCCC/C=C\C/C=C\CCCCC)OC(=O)CCCN(C)C]

and pharmaceutically acceptable salts thereof.

Other suitable cationic lipids for use in the LNPs, compositions, pharmaceutical compositions and methods of the present invention include ionizable cationic lipids as described in International Patent Publication WO 2013/149140, which is incorporated herein by reference. In some embodiments, the LNPs, compositions, pharmaceutical compositions and methods of the present invention include a cationic lipid of one of the following formulas:

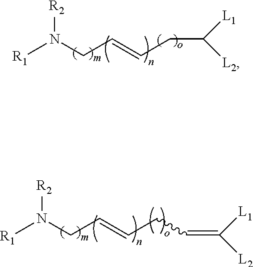

or a pharmaceutically acceptable salt thereof, wherein R₁ and R₂ are each independently selected from the group consisting of hydrogen, an optionally substituted, variably saturated or unsaturated C₁-C₂₀ alkyl and an optionally substituted, variably saturated or unsaturated C6-C₂₀ acyl; wherein L₁ and L₂ are each independently selected from the group consisting of hydrogen, an optionally substituted C₁-C₃₀ alkyl, an optionally substituted variably unsaturated C₁-C₃₀ alkenyl, and an optionally substituted C₁-C₃₀ alkynyl; wherein m and o are each independently selected from the group consisting of zero and any positive integer (e.g., where m is three); and wherein n is zero or any positive integer (e.g., where n is one). In some embodiments, the LNPs, compositions, pharmaceutical compositions and methods of the present invention include the cationic lipid (15Z, 18Z)—N,N-dimethyl-6-(9Z,12Z)-octadeca-9,12-dien-1-yl) tetracosa-15,18-dien-1-amine (“HGT5000”), having a compound structure of:

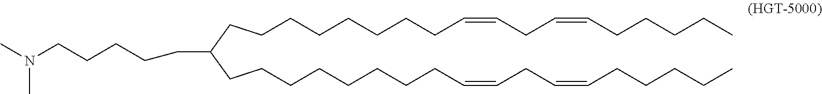 [SMILES: CCCCC/C=C\C/C=C\CCCCCCCCC(CCCCCCCC/C=C\C/C=C\CCCCC)CCCCCN(C)C]

and pharmaceutically acceptable salts thereof. In some embodiments, the LNPs, compositions, pharmaceutical compositions and methods of the present invention include the cationic lipid (15Z, 18Z)—N,N-dimethyl-6-((9Z,12Z)-octadeca-9,12-dien-1-yl) tetracosa-4,15,18-trien-1-amine (“HGT5001”), having a compound structure of:

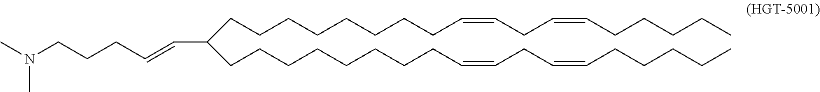 [SMILES: CCCCC/C=C\C/C=C\CCCCCCCCC(/C=C/CCCN(C)C)CCCCCCCC/C=C\C/C=C\CCCCC]

and pharmaceutically acceptable salts thereof. In some embodiments, the LNPs, compositions, pharmaceutical compositions and methods of the present invention include the cationic lipid and (15Z,18Z)—N,N-dimethyl-6-((9Z,12Z)-octadeca-9,12-dien-1-yl) tetracosa-5,15,18-trien-1-amine (“HGT5002”), having a compound structure of:

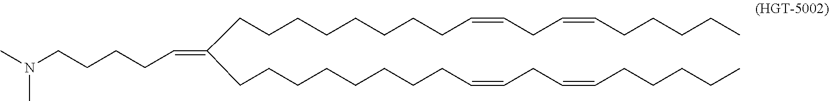 [SMILES: CCCCC/C=C\C/C=C\CCCCCCCCC(=CCCCCN(C)C)CCCCCCCC/C=C\C/C=C\CCCCC]

and pharmaceutically acceptable salts thereof.

Other suitable cationic lipids for use in the LNPs, compositions, pharmaceutical compositions and methods of the invention include cationic lipids described as aminoalcohol lipidoids in International Patent Publication WO 2010/053572, which is incorporated herein by reference. In some embodiments, the LNPs, compositions, pharmaceutical compositions and methods of the present invention include a cationic lipid having a compound structure of:

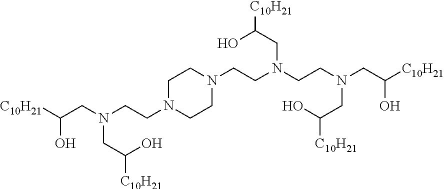

and pharmaceutically acceptable salts thereof.

Other suitable cationic lipids for use in the LNPs, compositions, pharmaceutical compositions and methods of the invention include the cationic lipids as described in International Patent Publication WO 2016/118725, which is incorporated herein by reference. In some embodiments, the LNPs, compositions, pharmaceutical compositions and methods of the present invention include a cationic lipid having a compound structure of:

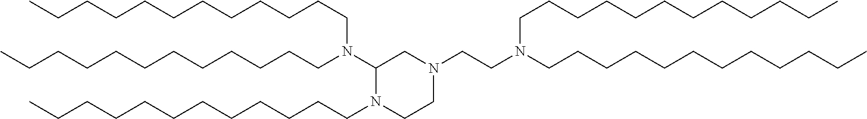 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable cationic lipids for use in the LNPs, compositions, pharmaceutical compositions and methods of the invention include the cationic lipids as described in International Patent Publication WO 2016/118724, which is incorporated herein by reference. In some embodiments, the LNPs, compositions, pharmaceutical compositions and methods of the present invention include a cationic lipid having a compound structure of:

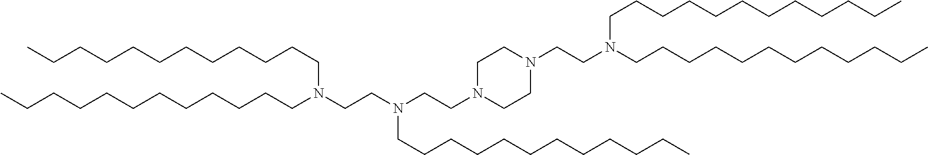 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable cationic lipids for use in the LNPs, compositions, pharmaceutical compositions and methods of the invention include a cationic lipid having the formula of 14,25-ditridecyl 15,18,21,24-tetraaza-octatriacontane, and pharmaceutically acceptable salts thereof.

Other suitable cationic lipids for use in the LNPs, compositions, pharmaceutical compositions and methods of the invention include the cationic lipids as described in International Patent Publications WO 2013/063468 and WO 2016/205691, each of which are incorporated herein by reference. In some embodiments, the LNPs, compositions, pharmaceutical compositions and methods of the present invention include a cationic lipid of the following formula:

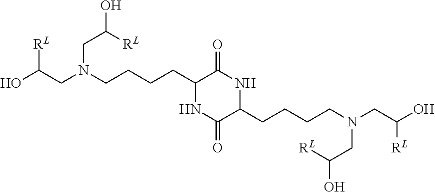

or pharmaceutically acceptable salts thereof, wherein each instance of R^{L} is independently optionally substituted C₆-C₄₀ alkenyl. In some embodiments, the LNPs, compositions, pharmaceutical compositions and methods of the present invention include a cationic lipid having a compound structure of:

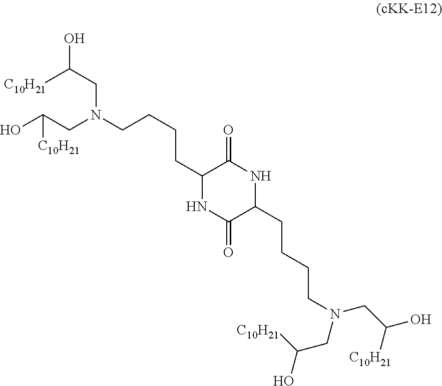

and pharmaceutically acceptable salts thereof. In some embodiments, the LNPs, compositions, pharmaceutical compositions and methods of the present invention include a cationic lipid having a compound structure of:

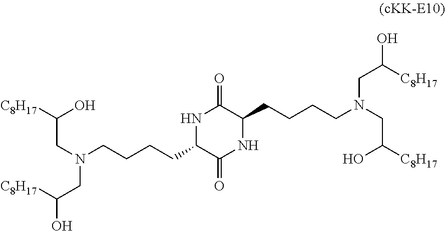

and pharmaceutically acceptable salts thereof. In some embodiments, the LNPs, compositions, pharmaceutical compositions and methods of the present invention include a cationic lipid having a compound structure of:

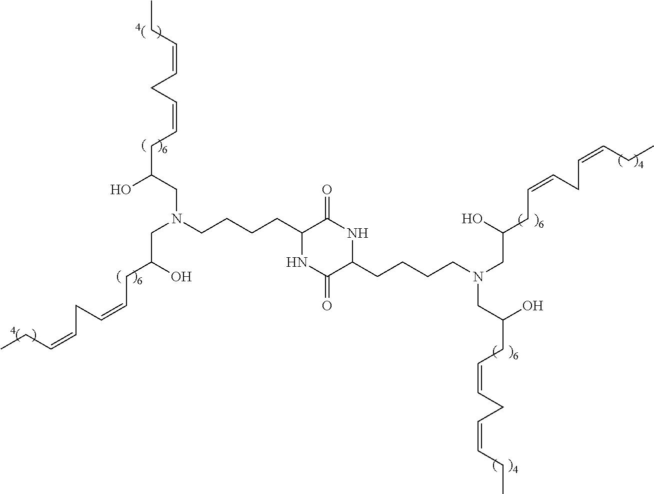

and pharmaceutically acceptable salts thereof. In some embodiments, the LNPs, compositions, pharmaceutical compositions and methods of the present invention include a cationic lipid having a compound structure of:

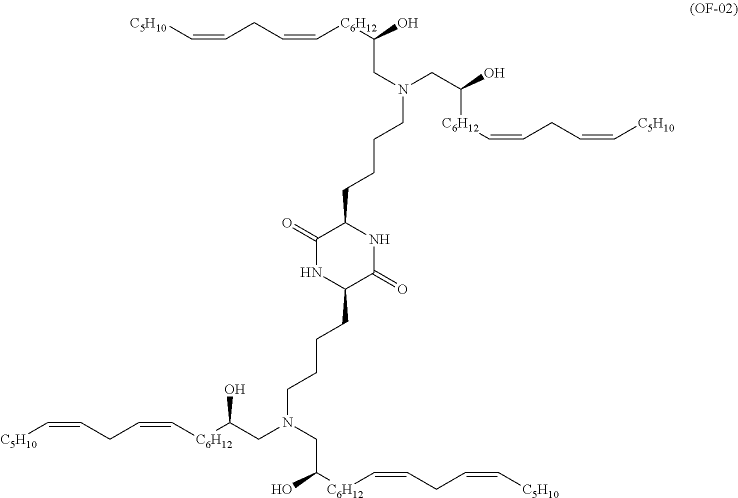

and pharmaceutically acceptable salts thereof. In some embodiments, the LNPs, compositions, pharmaceutical compositions and methods of the present invention include a cationic lipid having a compound structure of:

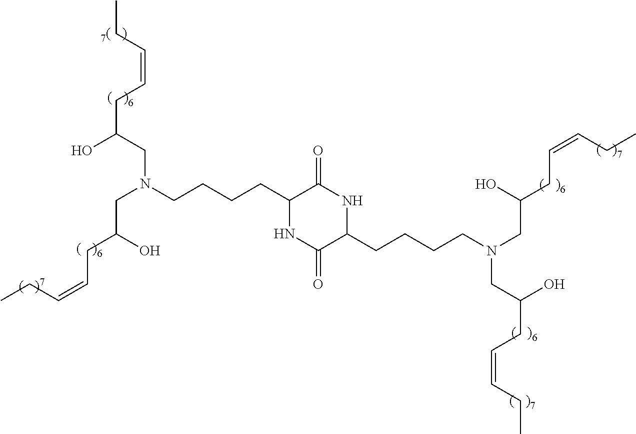

and pharmaceutically acceptable salts thereof. In some embodiments, the LNPs, compositions, pharmaceutical compositions and methods of the present invention include a cationic lipid having a compound structure of:

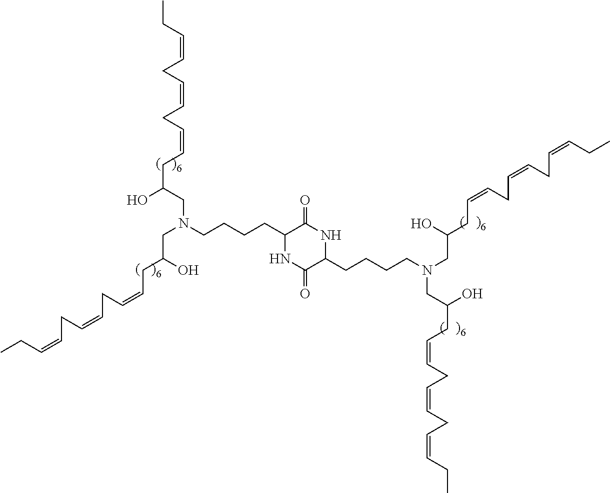

and pharmaceutically acceptable salts thereof.

Other suitable cationic lipids for use in the LNPs, compositions, pharmaceutical compositions and methods of the invention include the cationic lipids as described in International Patent Publication WO 2015/184256, which is incorporated herein by reference. In some embodiments, the LNPs, compositions, pharmaceutical compositions and methods of the present invention include a cationic lipid of the following formula:

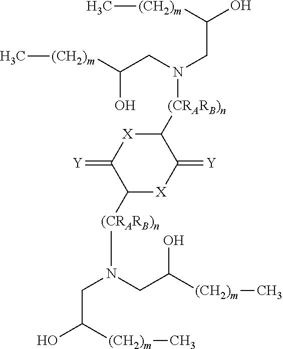

or a pharmaceutically acceptable salt thereof, wherein each X independently is O or S; each Y independently is O or S; each m independently is 0 to 20; each n independently is 1 to 6; each R_{A} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each R_{B} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen. In some embodiments, the LNPs, compositions, pharmaceutical compositions and methods of the present invention include a cationic lipid, “Target 23”, having a compound structure of:

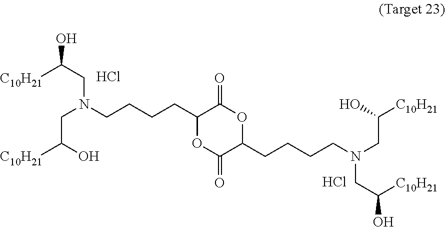

and pharmaceutically acceptable salts thereof.

Other suitable cationic lipids for use in the LNPs, compositions, pharmaceutical compositions and methods of the invention include the cationic lipids as described in International Patent Publication WO 2016/004202, which is incorporated herein by reference. In some embodiments, the LNPs, compositions, pharmaceutical compositions and methods of the present invention include a cationic lipid having the compound structure:

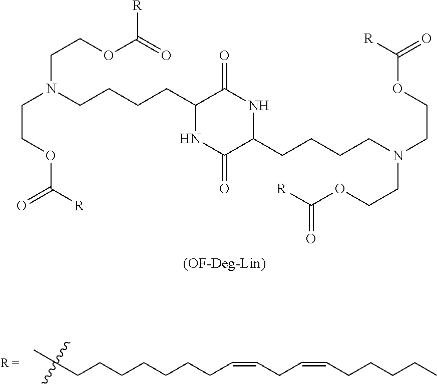

or a pharmaceutically acceptable salt thereof. In some embodiments, the LNPs, compositions, pharmaceutical compositions and methods of the present invention include a cationic lipid having the compound structure:

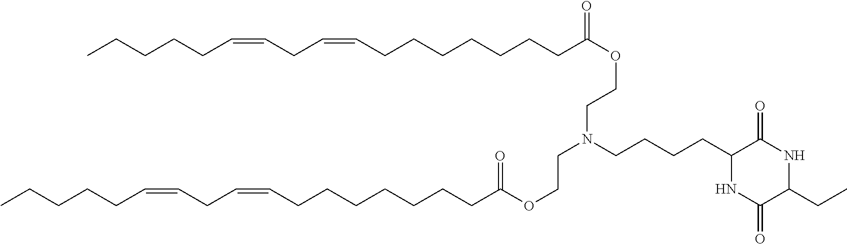 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof. In some embodiments, the LNPs, compositions, pharmaceutical compositions and methods of the present invention include a cationic lipid having the compound structure:

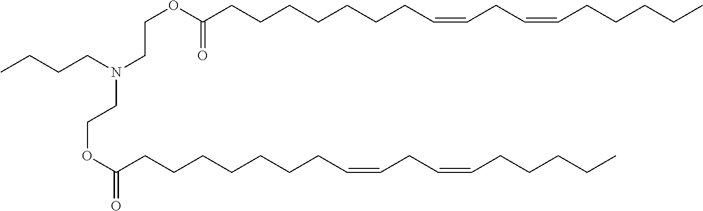 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable cationic lipids for use in the LNPs, compositions, pharmaceutical compositions and methods of the present invention include cationic lipids as described in International Patent Publication WO 2020/097384, which is incorporated herein by reference. In some embodiments, the LNPs, compositions, pharmaceutical compositions and methods of the present invention include a cationic lipid of the following formula:

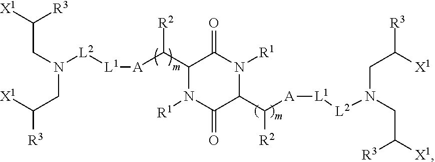

or a pharmaceutically acceptable salt thereof, wherein each R¹ and R² is independently H or C₁-C₆ aliphatic; each m is independently an integer having a value of 1 to 4; each A is independently a covalent bond or arylene; each L¹ is independently an ester, thioester, disulfide, or anhydride group; each L² is independently C₂-C₁₀ aliphatic; each X¹ is independently H or OH; and each R³ is independently C₆-C₂₀ aliphatic. In some embodiments, the LNPs, compositions, pharmaceutical compositions and methods of the present invention include a cationic lipid of the following formula:

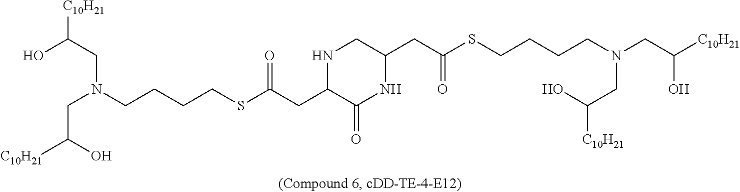

or a pharmaceutically acceptable salt thereof. In some embodiments, the LNPs, compositions, pharmaceutical compositions and methods of the present invention include a cationic lipid of the following formula:

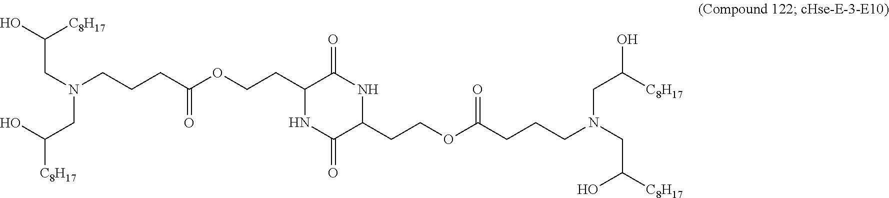

or a pharmaceutically acceptable salt thereof. In some embodiments, the LNPs, compositions, pharmaceutical compositions and methods of the present invention include a cationic lipid of the following formula:

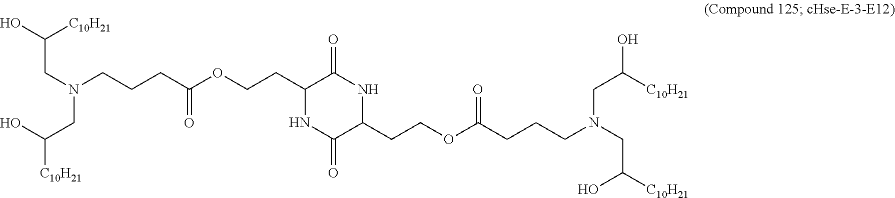

or a pharmaceutically acceptable salt thereof.

Other suitable cationic lipids for use in the pharmaceutical compositions and methods of the present invention include the cationic lipids as described in J. McClellan, M. C. King, Cell 2010, 141, 210-217 and in Whitehead et al., Nature Communications (2014) 5:4277, which is incorporated herein by reference. In some embodiments, the cationic lipids of the LNPs, compositions, pharmaceutical compositions and methods of the present invention include a cationic lipid having a compound structure of:

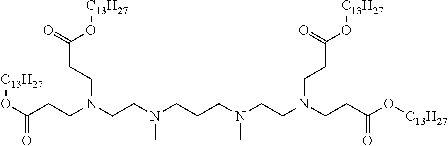

and pharmaceutically acceptable salts thereof.

Other suitable cationic lipids for use in the LNPs, compositions, pharmaceutical compositions and methods of the invention include the cationic lipids as described in International Patent Publication WO 2015/199952, which is incorporated herein by reference. In some embodiments, the LNPs, compositions, pharmaceutical compositions and methods of the present invention include a cationic lipid having the compound structure:

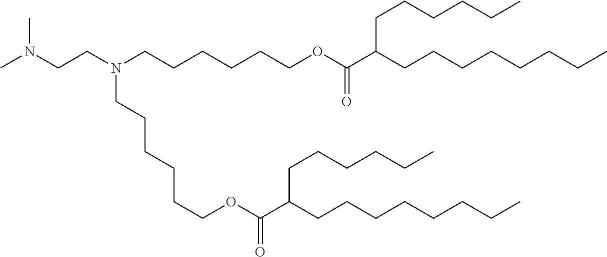 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof. In some embodiments, the LNPs, compositions, pharmaceutical compositions and methods of the present invention include a cationic lipid having the compound structure:

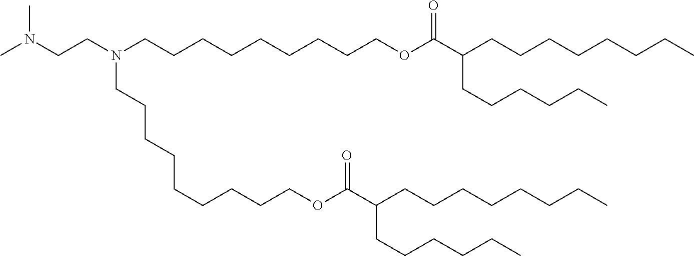 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof. In some embodiments, the LNPs, compositions, pharmaceutical compositions and methods of the present invention include a cationic lipid having the compound structure:

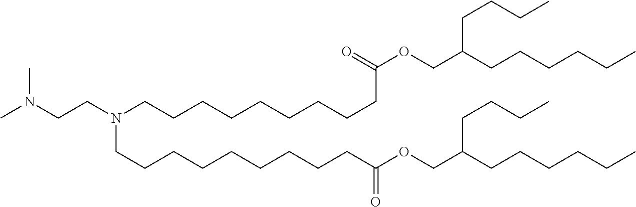 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof. In some embodiments, the LNPs, compositions, pharmaceutical compositions and methods of the present invention include a cationic lipid having the compound structure:

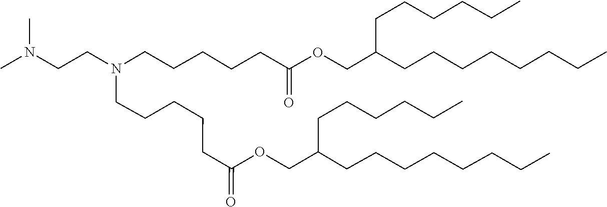 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof. In some embodiments, the LNPs, compositions, pharmaceutical compositions and methods of the present invention include a cationic lipid having the compound structure:

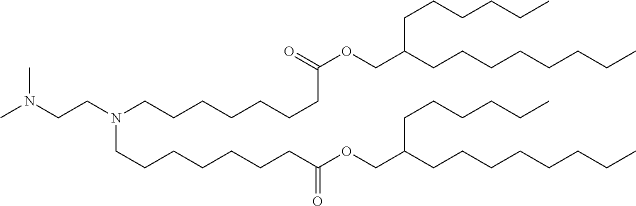 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof. In some embodiments, the LNPs, compositions, pharmaceutical compositions and methods of the present invention include a cationic lipid having the compound structure:

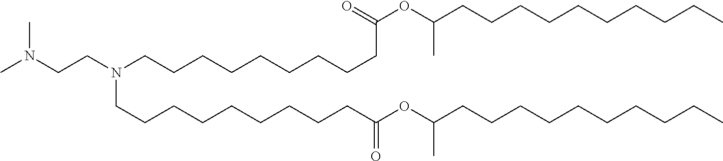 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof. In some embodiments, the LNPs, compositions, pharmaceutical compositions and methods of the present invention include a cationic lipid having the compound structure:

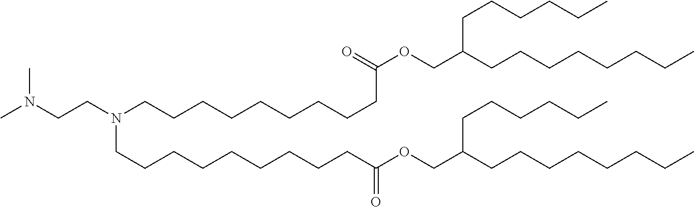 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof. In some embodiments, the LNPs, compositions, pharmaceutical compositions and methods of the present invention include a cationic lipid having the compound structure:

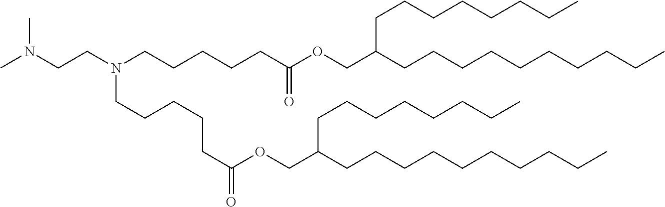 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof. In some embodiments, the LNPs, compositions, pharmaceutical compositions and methods of the present invention include a cationic lipid having the compound structure:

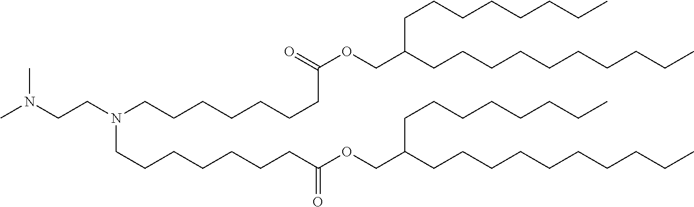 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof. In some embodiments, the LNPs, compositions, pharmaceutical compositions and methods of the present invention include a cationic lipid having the compound structure:

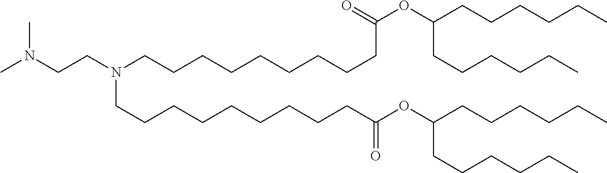 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof. In some embodiments, the LNPs, compositions, pharmaceutical compositions and methods of the present invention include a cationic lipid having the compound structure:

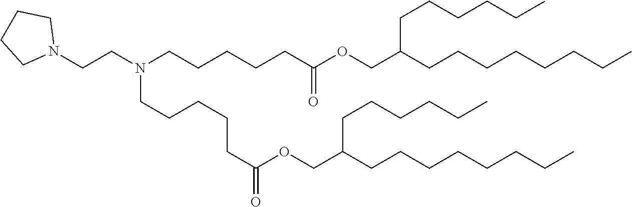 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof. In some embodiments, the LNPs, compositions, pharmaceutical compositions and methods of the present invention include a cationic lipid having the compound structure:

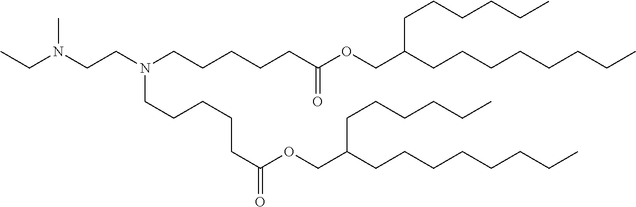 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof. In some embodiments, the LNPs, compositions, pharmaceutical compositions and methods of the present invention include a cationic lipid having the compound structure:

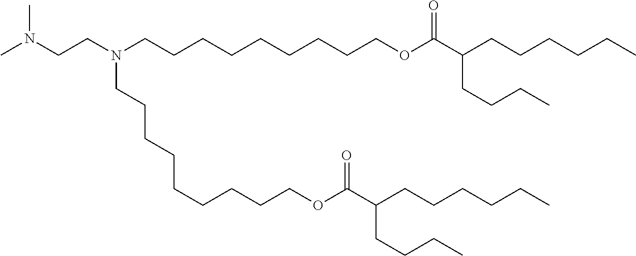 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable cationic lipids for use in the LNPs, compositions, pharmaceutical compositions and methods of the invention include the cationic lipids as described in International Patent Publication WO 2017/004143, which is incorporated herein by reference. In some embodiments, the LNPs, compositions, pharmaceutical compositions and methods of the present invention include a cationic lipid having the compound structure:

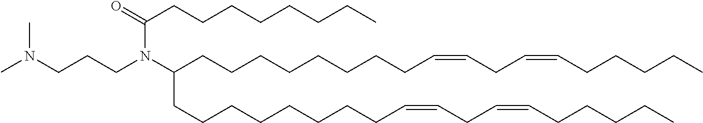 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof. In some embodiments, the LNPs, compositions, pharmaceutical compositions and methods of the present invention include a cationic lipid having the compound structure:

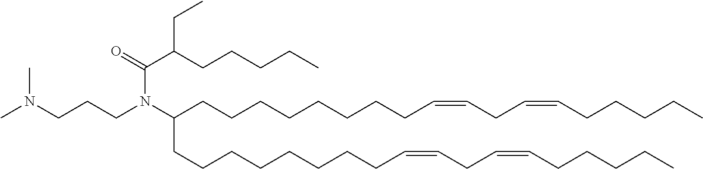 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof. In some embodiments, the LNPs, compositions, pharmaceutical compositions and methods of the present invention include a cationic lipid having the compound structure:

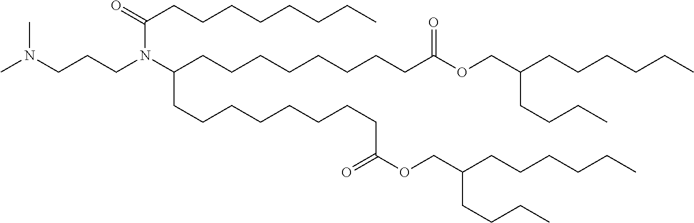 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof. In some embodiments, the LNPs, compositions, pharmaceutical compositions and methods of the present invention include a cationic lipid having the compound structure:

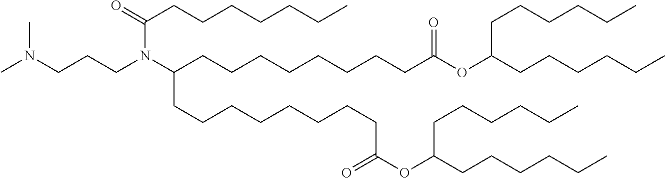 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the LNPs, compositions, pharmaceutical compositions and methods of the present invention include a cationic lipid having the compound structure:

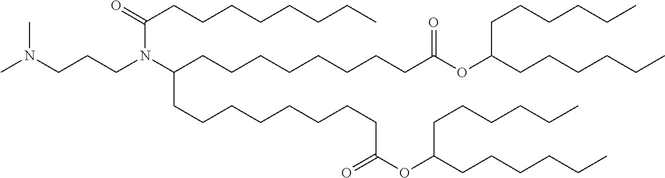 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the LNPs, compositions, pharmaceutical compositions and methods of the present invention include a cationic lipid having the compound structure:

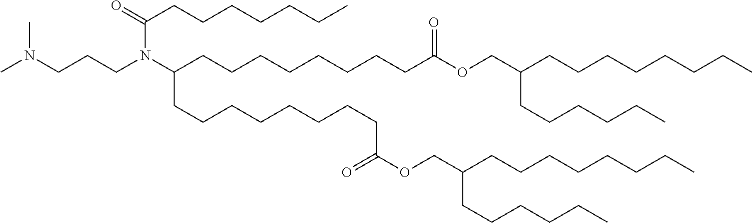 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof. In some embodiments, the LNPs, compositions, pharmaceutical compositions and methods of the present invention include a cationic lipid having the compound structure:

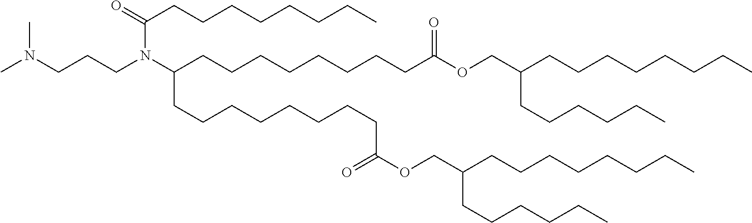 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof. In some embodiments, the LNPs, compositions, pharmaceutical compositions and methods of the present invention include a cationic lipid having the compound structure:

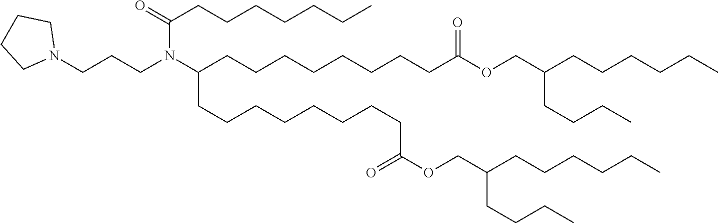 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the LNPs, compositions, pharmaceutical compositions and methods of the present invention include a cationic lipid having the compound structure:

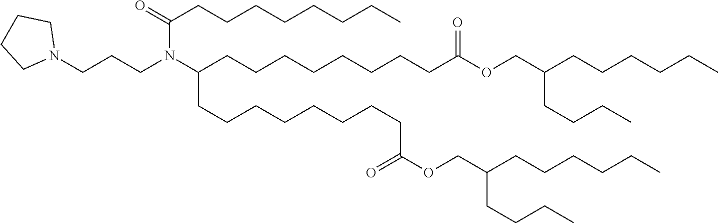 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the LNPs, compositions, pharmaceutical compositions and methods of the present invention include a cationic lipid having the compound structure:

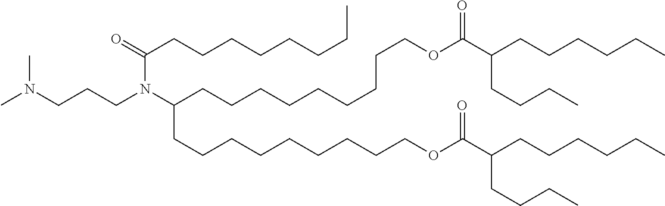 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof. In some embodiments, the LNPs, compositions, pharmaceutical compositions and methods of the present invention include a cationic lipid having the compound structure:

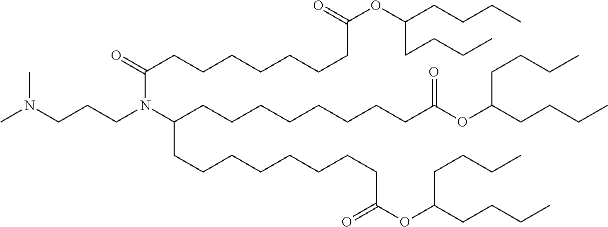 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof. In some embodiments, the LNPs, compositions, pharmaceutical compositions and methods of the present invention include a cationic lipid having the compound structure:

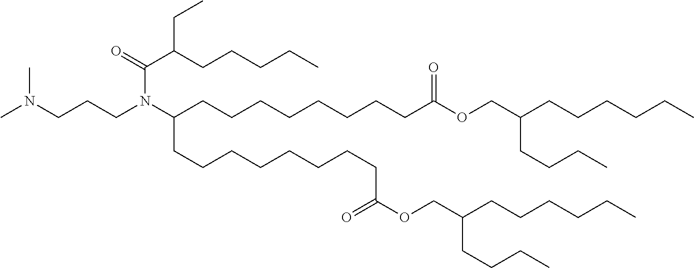 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof. In some embodiments, the LNPs, compositions, pharmaceutical compositions and methods of the present invention include a cationic lipid having the compound structure:

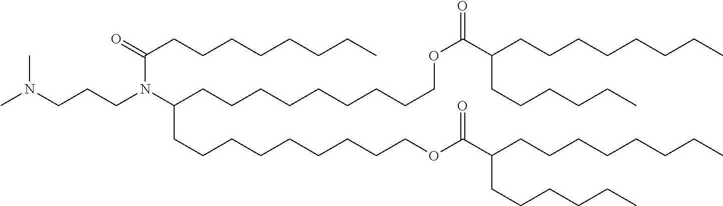 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof. In some embodiments, the LNPs, compositions, pharmaceutical compositions and methods of the present invention include a cationic lipid having the compound structure:

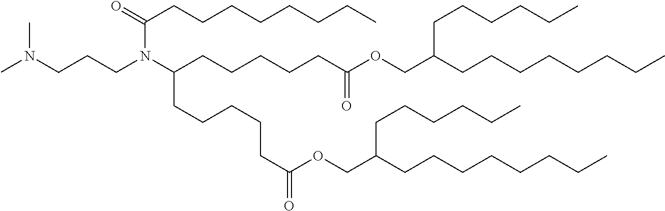 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof. In some embodiments, the LNPs, compositions, pharmaceutical compositions and methods of the present invention include a cationic lipid having the compound structure:

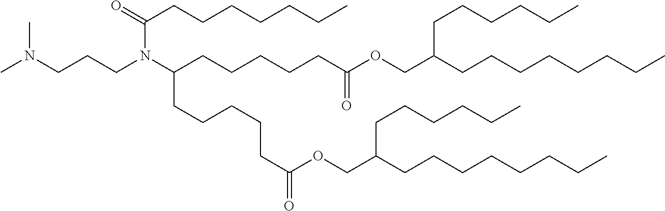 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

and pharmaceutically acceptable salts thereof. In some embodiments, the LNPs, compositions, pharmaceutical compositions and methods of the present invention include a cationic lipid having the compound structure:

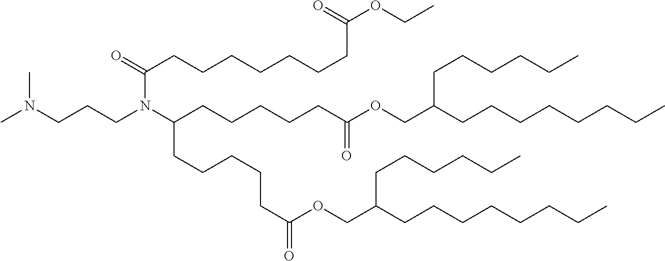 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof. In some embodiments, the LNPs, compositions, pharmaceutical compositions and methods of the present invention include a cationic lipid having the compound structure:

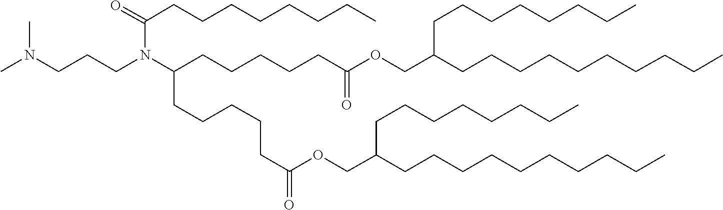 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable cationic lipids for use in the LNPs, compositions, pharmaceutical compositions and methods of the invention include the cationic lipids as described in International Patent Publication WO 2017/075531, which is incorporated herein by reference. In some embodiments, the LNPs, compositions, pharmaceutical compositions and methods of the present invention include a cationic lipid of the following formula:

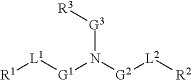

or a pharmaceutically acceptable salt thereof, wherein one of L¹ or L² is —O(C═O)—, —(C═O)O—, —C(═O)—, —O—, —S(O)ₓ, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹ or L² is —O(C═O)—, —(C═O)O—, —C(═O)—, —O—, —S(O)ₓ, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond; G¹ and G² are each independently unsubstituted C₁-C₁₂ alkylene or C₁-C₁₂ alkenylene; G³ is C₁-C₂₄ alkylene, C₁-C₂₄ alkenylene, C₃-C₈ cycloalkylene, C₃-C₈ cycloalkenylene; R^{a} is H or C₁-C₁₂ alkyl; R¹ and R² are each independently C₆-C₂₄ alkyl or C₆-C₂₄ alkenyl; R³ is H, OR′, CN, —C(═O)OR⁴, —OC(═O)R⁴ or —NR⁵C(═O)R⁴; R⁴ is C₁-C₁₂ alkyl; R⁵ is H or C₁-C₆ alkyl; and x is 0, 1 or 2.

Other suitable cationic lipids for use in the LNPs, compositions, pharmaceutical compositions and methods of the invention include the cationic lipids as described in International Patent Publication WO 2017/117528, which is incorporated herein by reference. In some embodiments, the LNPs, compositions, pharmaceutical compositions and methods of the present invention include a cationic lipid having the compound structure:

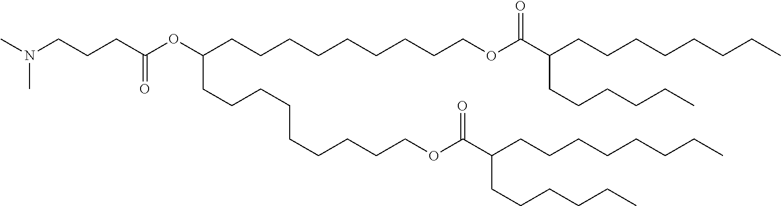 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof. In some embodiments, the LNPs, compositions, pharmaceutical compositions and methods of the present invention include a cationic lipid having the compound structure:

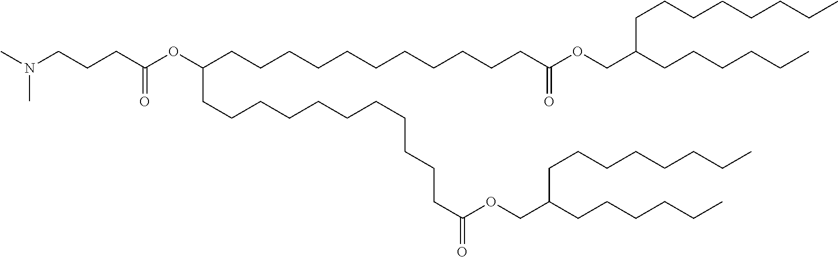 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof. In some embodiments, the LNPs, compositions, pharmaceutical compositions and methods of the present invention include a cationic lipid having the compound structure:

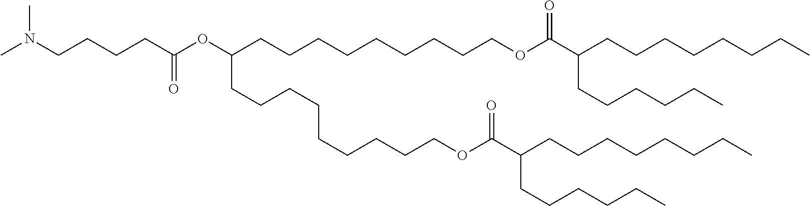 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable cationic lipids for use in the LNPs, compositions, pharmaceutical compositions and methods of the invention include the cationic lipids as described in International Patent Publication WO 2017/049245, which is incorporated herein by reference. In some embodiments, the cationic lipids of the LNPs, compositions, pharmaceutical compositions and methods of the present invention include a compound of one of the following formulas:

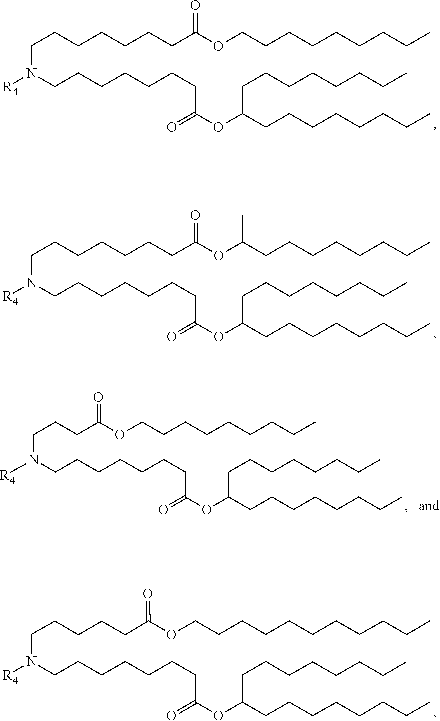

and pharmaceutically acceptable salts thereof. For any one of these four formulas, R₄ is independently selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3. In some embodiments, the LNPs, compositions, pharmaceutical compositions and methods of the present invention include a cationic lipid having a compound structure of:

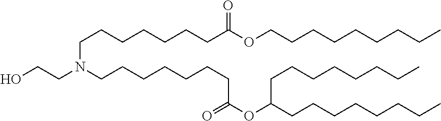 [SMILES: CCCCCCCCCOC(=O)CCCCCCCN(CCO)CCCCCCCC(=O)OC(CCCCCCCC)CCCCCCCC]

and pharmaceutically acceptable salts thereof. In some embodiments, the LNPs, compositions, pharmaceutical compositions and methods of the present invention include a cationic lipid having a compound structure of:

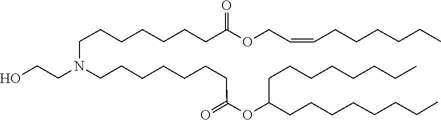 [SMILES: CCCCCC/C=C\COC(=O)CCCCCCCN(CCO)CCCCCCCC(=O)OC(CCCCCCCC)CCCCCCCC]

and pharmaceutically acceptable salts thereof. In some embodiments, the LNPs, compositions, pharmaceutical compositions and methods of the present invention include a cationic lipid having a compound structure of:

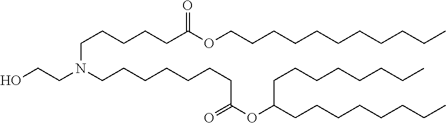 [SMILES: CCCCCCCCCCCOC(=O)CCCCCN(CCO)CCCCCCCC(=O)OC(CCCCCCCC)CCCCCCCC]

and pharmaceutically acceptable salts thereof. In some embodiments, the LNPs, compositions, pharmaceutical compositions and methods of the present invention include a cationic lipid having a compound structure of:

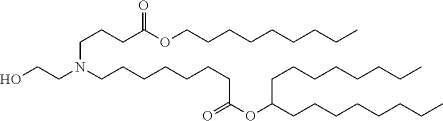 [SMILES: CCCCCCCCCOC(=O)CCCN(CCO)CCCCCCCC(=O)OC(CCCCCCCC)CCCCCCCC]

and pharmaceutically acceptable salts thereof.

Other suitable cationic lipids for use in the LNPs, compositions, pharmaceutical compositions and methods of the invention include the cationic lipids as described in International Patent Publication WO 2017/173054 and WO 2015/095340, each of which is incorporated herein by reference. In some embodiments, the LNPs, compositions, pharmaceutical compositions and methods of the present invention include a cationic lipid having a compound structure of:

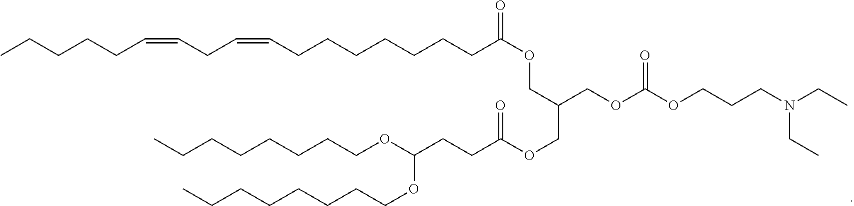 [SMILES: CCCCC/C=C\C/C=C\CCCCCCCC(=O)OCC(COC(=O)CCC(OCCCCCCCC)OCCCCCCCC)COC(=O)OCCCN(CC)CC]

and pharmaceutically acceptable salts thereof. In some embodiments, the LNPs, compositions, pharmaceutical compositions and methods of the present invention include a cationic lipid having a compound structure of:

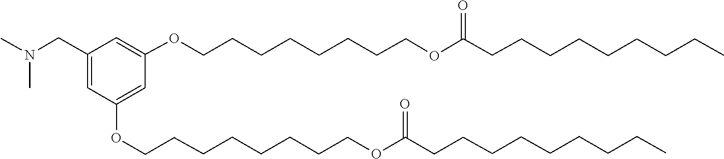 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof. In some embodiments, the LNPs, compositions, pharmaceutical compositions and methods of the present invention include a cationic lipid having a compound structure of:

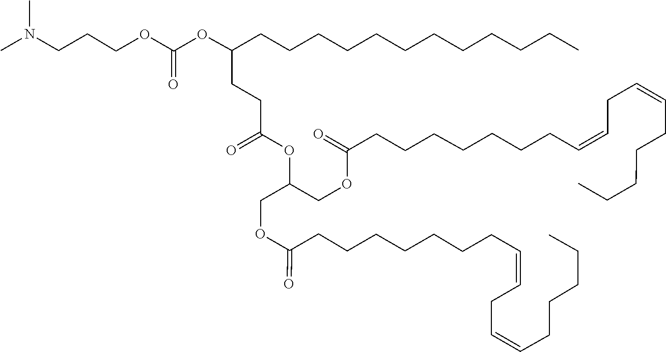 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof. In some embodiments, the LNPs, compositions, pharmaceutical compositions and methods of the present invention include a cationic lipid having a compound structure of:

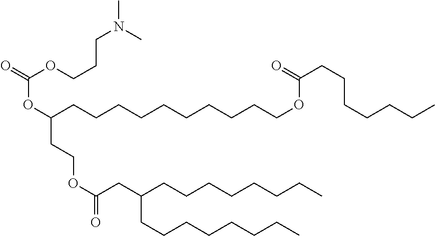 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

Other suitable cationic lipids for use in the LNPs, compositions, pharmaceutical compositions and methods of the present invention include cationic lipids as described in U.S. Provisional Patent Application Ser. No. 63/082,090, filed on Sep. 23, 2020, which is incorporated herein by reference. In some embodiments, the pharmaceutical compositions and methods of the present invention include a cationic lipid having a compound structure of:

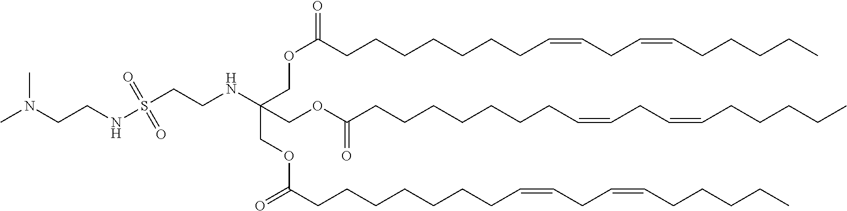 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCN(C)C]

(GL-TES-SA-DME-E18-2) and pharmaceutically acceptable salts thereof.

In some embodiments, the LNPs, compositions, pharmaceutical compositions and methods of the present invention include a cationic lipid having a compound structure of:

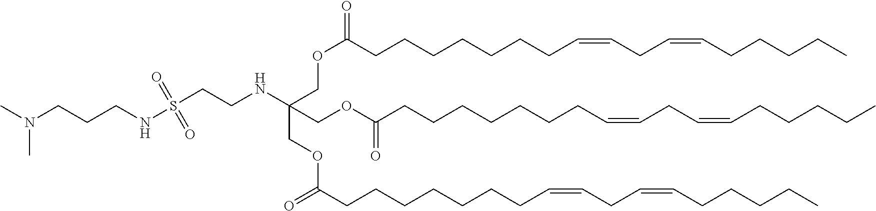 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCCN(C)C]

(GL-TES-SA-DMP-E18-2) and pharmaceutically acceptable salts thereof.

Other suitable cationic lipids for use in the LNPs, compositions, pharmaceutical compositions and methods of the present invention include cationic lipids as described in U.S. Provisional Patent Application Ser. No. 63/003,698, filed on Apr. 1, 2020, which is incorporated herein by reference. In some embodiments, the LNPs, compositions, pharmaceutical compositions and methods of the present invention include a cationic lipid having a compound structure of:

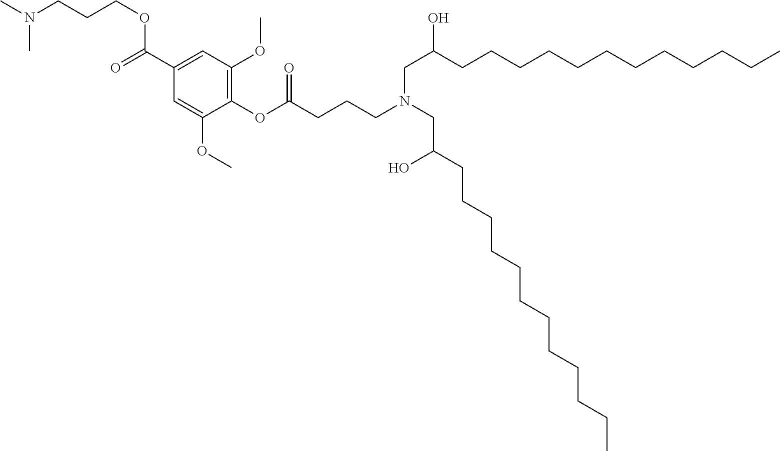 [SMILES: CCCCCCCCCCCCC(O)CN(CCCC(=O)Oc1c(OC)cc(C(=O)OCCCN(C)C)cc1OC)CC(O)CCCCCCCCCCCC]

(SY-3-E14-DMAPr) and pharmaceutically acceptable salts thereof.

Other suitable cationic lipids for use in the LNPs, compositions, pharmaceutical compositions and methods of the present invention include cationic lipids as described in U.S. Provisional Patent Application Ser. No. 63/082,101, filed on Sep. 23, 2020, which is incorporated herein by reference. In some embodiments, the LNPs, compositions, pharmaceutical compositions and methods of the present invention include a cationic lipid having a compound structure of:

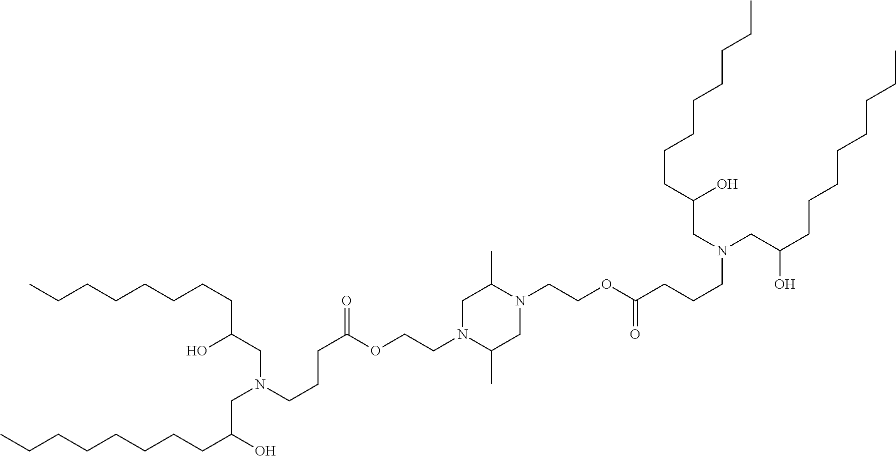 [SMILES: CCCCCCCCC(O)CN(CCCC(=O)OCCN1CC(C)N(CCOC(=O)CCCN(CC(O)CCCCCCCC)CC(O)CCCCCCCC)CC1C)CC(O)CCCCCCCC]

(HEP-E3-E10) and pharmaceutically acceptable salts thereof.

In some embodiments, the LNPs, compositions, pharmaceutical compositions and methods of the present invention include a cationic lipid having a compound structure of:

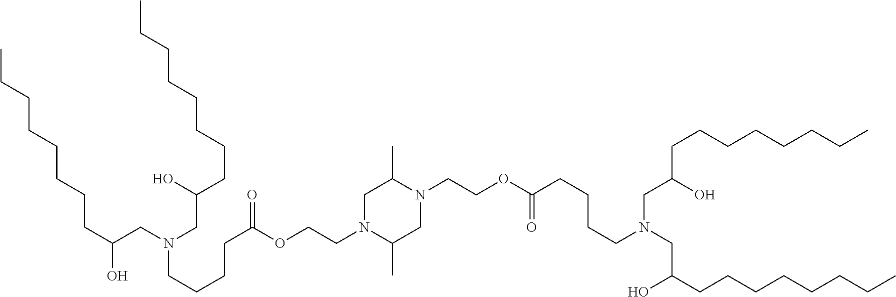 [SMILES: CCCCCCCCC(O)CN(CCCCC(=O)OCCN1CC(C)N(CCOC(=O)CCCCN(CC(O)CCCCCCCC)CC(O)CCCCCCCC)CC1C)CC(O)CCCCCCCC]

(HEP-E4-E10) and pharmaceutically acceptable salts thereof.

Other suitable cationic lipids for use in the LNPs, compositions, pharmaceutical compositions and methods of the present invention include cationic lipids as described in U.S. Provisional Patent Application Ser. No. 62/864,818, filed on Jun. 21, 2019, which is incorporated herein by reference. In some embodiments, the LNPs, compositions, pharmaceutical compositions and methods of the present invention include a cationic lipid having a compound structure according to the following formula:

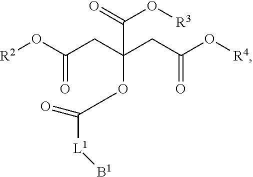

or a pharmaceutically acceptable salt thereof, wherein each of R², R³, and R⁴ is independently C₆-C₃₀ alkyl, C₆-C₃₀ alkenyl, or C₆-C₃₀ alkynyl; L¹ is C₁-C₃₀ alkylene; C₂-C₃₀ alkenylene; or C₂-C₃₀ alkynylene and B¹ is an ionizable nitrogen-containing group. In embodiments, L¹ is C₁-C₁₀ alkylene. In embodiments, L¹ is unsubstituted C₁-C₁₀ alkylene. In embodiments, L¹ is (CH₂)₂, (CH₂)₃, (CH₂)₄, or (CH₂)₅. In embodiments, L¹ is (CH₂), (CH₂)₆, (CH₂)₇, (CH₂)₈, (CH₂)₉, or (CH₂)₁₀. In embodiments, B¹ is independently NH₂, guanidine, amidine, a mono- or dialkylamine, 5- to 6-membered nitrogen-containing heterocycloalkyl, or 5- to 6-membered nitrogen-containing heteroaryl. In embodiments, B¹

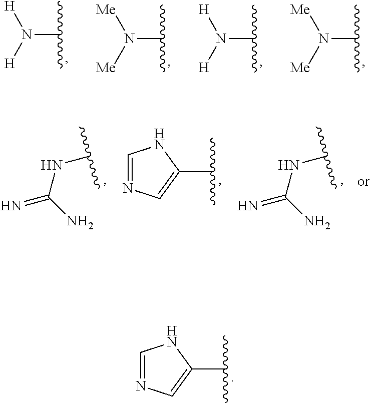

In embodiments, B¹ is

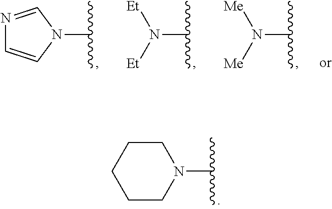

In embodiments, B¹ is

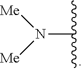 [SMILES: *N(C)C]

In embodiments, each of R², R³, and R⁴ is independently unsubstituted linear C₆-C₂₂ alkyl, unsubstituted linear C₆-C₂₂ alkenyl, unsubstituted linear C₆-C₂₂ alkynyl, unsubstituted branched C₆-C₂₂ alkyl, unsubstituted branched C₆-C₂₂ alkenyl, or unsubstituted branched C₆-C₂₂ alkynyl. In embodiments, each of R², R³, and R⁴ is unsubstituted C₆-C₂₂ alkyl. In embodiments, each of R², R³, and R⁴ is —C₆H₁₃, —C₇H₁₅, —C₈H₁₇, —C₉H₁₉, —C₁₀H₂₁, —C₁₁H₂₃, —C₁₂H₂₅, —C₁₃H₂₇, —C₁₄H₂₉, —C₁₅H₃₁, —C₁₆H₃₃, —C₁₇H₃₅, —C₁₈H₃₇, —C₁₉H₃₉, —C₂₀H₄₁, —C₂₁H₄₃, —C₂₂H₄₅, —C₂₃H₄₇, —C₂₄H₄₉, or —C₂₅H₅₁. In embodiments, each of R², R³, and R⁴ is independently C₆-C₁₂ alkyl substituted by —O(CO)R⁵ or —C(O)OR⁵, wherein R⁵ is unsubstituted C₆-C₁₄ alkyl. In embodiments, each of R², R³, and R⁴ is unsubstituted C₆-C₂₂ alkenyl. In embodiments, each of R², R³, and R⁴ is —(CH₂)₄CH═CH₂, —(CH₂)₅CH═CH₂, —(CH₂)₆CH═CH₂, —(CH₂)₇CH═CH₂, —(CH₂)₈CH═CH₂, —(CH₂)₉CH═CH₂, —(CH₂)₁₀CH═CH₂, —(CH₂)₁₁CH═CH₂, —(CH₂)₁₂CH═CH₂, —(CH₂)₁₃CH═CH₂, —(CH₂)₁₄CH═CH₂, —(CH₂)₁₅CH═CH₂, —(CH₂)₁₆CH═CH₂, —(CH₂)₁₇CH═CH₂, —(CH₂)₁₈CH═CH₂, —(CH₂)₇CH═CH(CH₂)₃CH₃, —(CH₂)₇CH═CH(CH₂)₅CH₃, —(CH₂)₄CH═CH(CH₂)₈CH₃, —(CH₂)₇CH═CH(CH₂)₇CH₃, —(CH₂)₆CH═CHCH₂CH═CH(CH₂)₄CH₃, —(CH₂)₇CH═CHCH₂CH═CH(CH₂)₄CH₃, —(CH₂)₇CH═CHCH₂CH═CHCH₂CH═CHCH₂CH₃, —(CH₂)₃CH═CHCH₂CH═CHCH₂CH═CHCH₂CH═CH(CH₂)₄CH₃, —(CH₂)₃CH═CHCH₂CH═CHCH₂CH═CHCH₂CH═CHCH₂CH═CHCH₂CH₃, —(CH₂)₁₁CH═CH(CH₂)₇CH₃, or —(CH₂)₂CH═CHCH₂CH═CHCH₂CH═CHCH₂CH═CHCH₂CH═CHCH₂CH═CHCH₂CH₃.
In embodiments, said C₆-C₂₂ alkenyl is a monoalkenyl, a dienyl, or a trienyl. In embodiments, each of R², R³, and R⁴ is

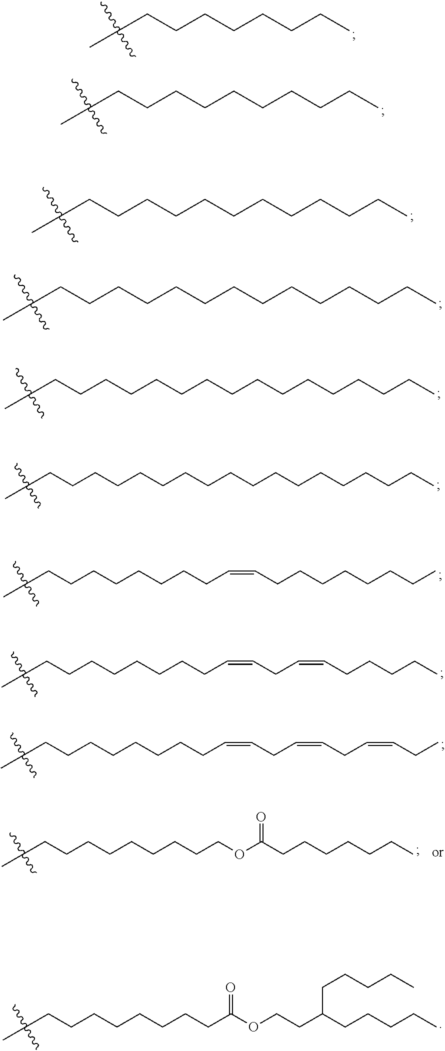

In some embodiments, the LNPs, compositions, pharmaceutical compositions and methods of the present invention include a cationic lipid having a compound structure of:

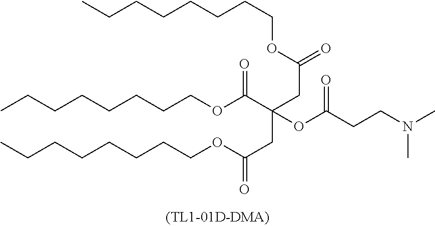 [SMILES: CCCCCCCCOC(=O)CC(CC(=O)OCCCCCCCC)(OC(=O)CCN(C)C)C(=O)OCCCCCCCC]

and pharmaceutically acceptable salts thereof. In some embodiments, the LNPs, compositions, pharmaceutical compositions and methods of the present invention include a cationic lipid having a compound structure of:

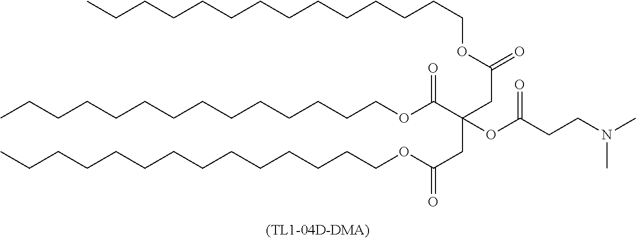 [SMILES: CCCCCCCCCCCCCCOC(=O)CC(CC(=O)OCCCCCCCCCCCCCC)(OC(=O)CCN(C)C)C(=O)OCCCCCCCCCCCCCC]

and pharmaceutically acceptable salts thereof. In some embodiments, the LNPs, compositions, pharmaceutical compositions and methods of the present invention include a cationic lipid having a compound structure of:

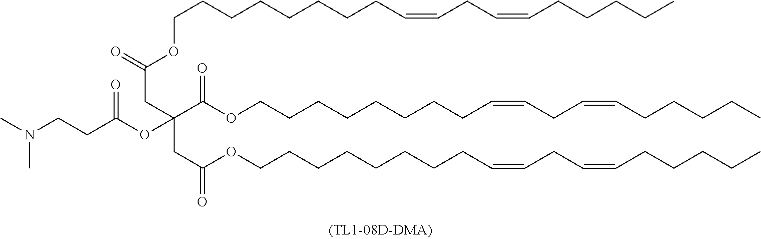 [SMILES: CCCCC/C=C\C/C=C\CCCCCCCCOC(=O)CC(CC(=O)OCCCCCCCC/C=C\C/C=C\CCCCC)(OC(=O)CCN(C)C)C(=O)OCCCCCCCC/C=C\C/C=C\CCCCC]

and pharmaceutically acceptable salts thereof. In some embodiments, the LNPs, compositions, pharmaceutical compositions and methods of the present invention include a cationic lipid having a compound structure of:

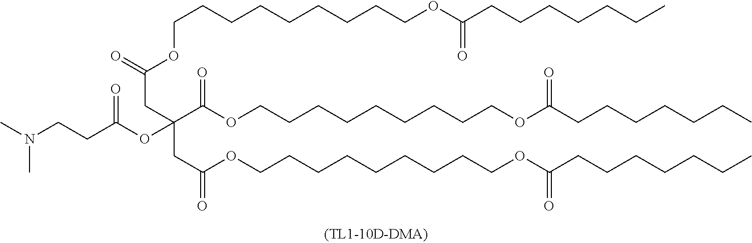 [SMILES: CCCCCCCC(=O)OCCCCCCCCCOC(=O)CC(CC(=O)OCCCCCCCCCOC(=O)CCCCCCC)(OC(=O)CCN(C)C)C(=O)OCCCCCCCCCOC(=O)CCCCCCC]

and pharmaceutically acceptable salts thereof.

Other suitable cationic lipids for use in the LNPs, compositions, pharmaceutical compositions and methods of the present invention include cleavable cationic lipids as described in International Patent Publication WO 2012/170889, which is incorporated herein by reference. In some embodiments, the LNPs, compositions, pharmaceutical compositions and methods of the present invention include a cationic lipid of the following formula:

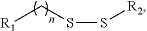

wherein R₁ is selected from the group consisting of imidazole, guanidinium, amino, imine, enamine, an optionally-substituted alkyl amino (e.g., an alkyl amino such as dimethylamino) and pyridyl; wherein R₂ is selected from the group consisting of one of the following two formulas:

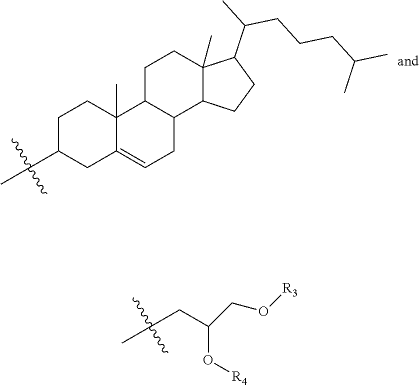

and wherein R₃ and R₄ are each independently selected from the group consisting of an optionally substituted, variably saturated or unsaturated C₆-C₂₀ alkyl and an optionally substituted, variably saturated or unsaturated C₆-C₂₀ acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more). In some embodiments, the LNPs, compositions, pharmaceutical compositions and methods of the present invention include a cationic lipid, “HGT4001”, having a compound structure of:

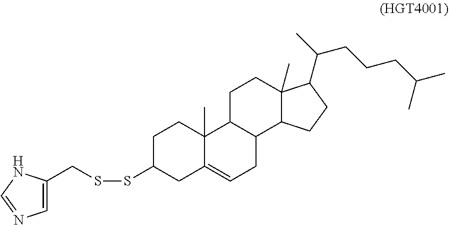 [SMILES: CC(C)CCCC(C)C1CCC2C3CC=C4CC(SSCc5cnc[nH]5)CCC4(C)C3CCC12C]

and pharmaceutically acceptable salts thereof. In some embodiments, the LNPs, compositions, pharmaceutical compositions and methods of the present invention include a cationic lipid, “HGT4002”, having a compound structure of:

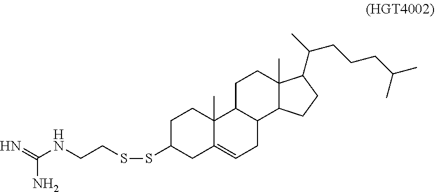 [SMILES: CC(C)CCCC(C)C1CCC2C3CC=C4CC(SSCCNC(=N)N)CCC4(C)C3CCC12C]

and pharmaceutically acceptable salts thereof. In some embodiments, the LNPs, compositions, pharmaceutical compositions and methods of the present invention include a cationic lipid, “HGT4003,” having a compound structure of:

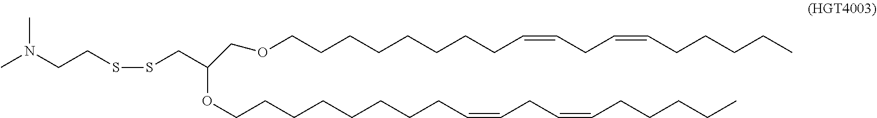 [SMILES: CCCCC/C=C\C/C=C\CCCCCCCCOCC(CSSCCN(C)C)OCCCCCCCC/C=C\C/C=C\CCCCC]

and pharmaceutically acceptable salts thereof. In some embodiments, the LNPs, compositions, pharmaceutical compositions and methods of the present invention include a cationic lipid, “HGT4004,” having a compound structure of:

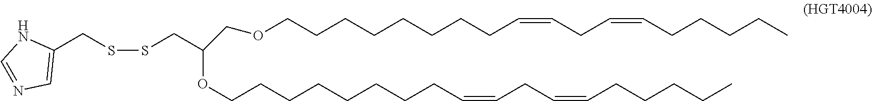 [SMILES: CCCCC/C=C\C/C=C\CCCCCCCCOCC(CSSCc1cnc[nH]1)OCCCCCCCC/C=C\C/C=C\CCCCC]

and pharmaceutically acceptable salts thereof. In some embodiments, the LNPs, compositions, pharmaceutical compositions and methods of the present invention include a cationic lipid “HGT4005,” having a compound structure of:

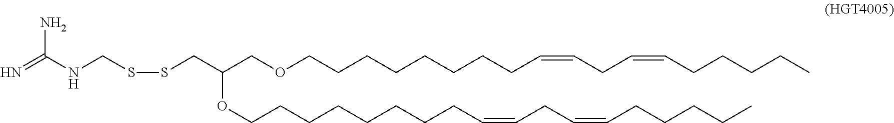 [SMILES: CCCCC/C=C\C/C=C\CCCCCCCCOCC(CSSCNC(=N)N)OCCCCCCCC/C=C\C/C=C\CCCCC]

and pharmaceutically acceptable salts thereof.

Other suitable cationic lipids for use in the LNPs, compositions, pharmaceutical compositions and methods of the present invention include cleavable cationic lipids as described in International Patent Publication WO 2019/222424, and incorporated herein by reference. In some embodiments, the LNPs, compositions, pharmaceutical compositions and methods of the present invention include a cationic lipid that is any of general formulas or any of structures (1a)-(21a) and (1b)-(21b) and (22)-(237) described in International Patent Publication WO 2019/222424. In some embodiments, the LNPs, compositions, pharmaceutical compositions and methods of the present invention include a cationic lipid that has a structure according to Formula (I′),

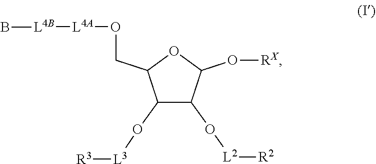

wherein:
R^{X} is independently —H, -L¹-R¹, or -L⁵A-L^{5B}-B′;
each of L¹, L², and L³ is independently a covalent bond, —C(O)—, —C(O)O—, —C(O)S—, or —C(O)NR^{L}—;
each L^{4A} and L^{5A} is independently —C(O)—, —C(O)—, or —C(O)NR^{L}—;
each L^{4B} and L^{5B} is independently C₁-C₂₀ alkylene; C₂-C₂₀ alkenylene; or C₂-C₂₀ alkynylene;
each B and B′ is NR⁴R⁵ or a 5- to 10-membered nitrogen-containing heteroaryl;
each R¹, R², and R³ is independently C₆-C₃₀ alkyl, C₆-C₃₀ alkenyl, or C₆-C₃₀ alkynyl;
each R⁴ and R⁵ is independently hydrogen, C₁-C₁₀ alkyl; C₂-C₁₀ alkenyl; or C₂-C₁₀ alkynyl; and
each R^{L} is independently hydrogen, C₁-C₂₀ alkyl, C₂-C₂₀ alkenyl, or C₂-C₂₀ alkynyl.
   In some embodiments, the LNPs, compositions, pharmaceutical compositions and methods of the present invention include a cationic lipid that is Compound (139) of International Patent Publication No. WO 2019/222424, having a compound structure of:

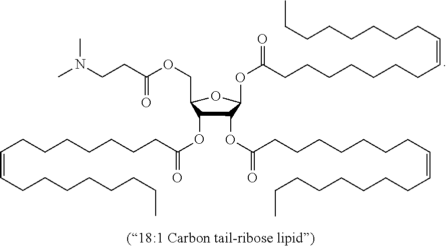

In some embodiments, the LNPs, compositions, pharmaceutical compositions and methods of the present invention include a cationic lipid that is RL3-DMA-07D having a compound structure of:

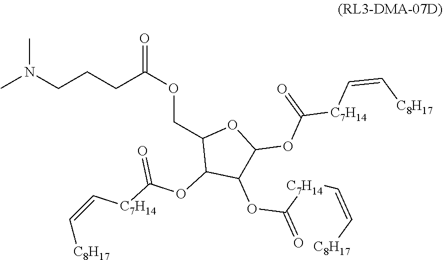

and pharmaceutically acceptable salts thereof.

In some embodiments, the LNPs, compositions, pharmaceutical compositions and methods of the present invention include a cationic lipid that is RL2-DMP-07D having a compound structure of:

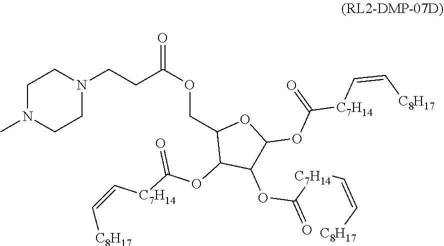

and pharmaceutically acceptable salts thereof.

In some embodiments, the LNPs, compositions, pharmaceutical compositions and methods of the present invention include the cationic lipid GL-HEPES-E3-E10-DS-3-E18-1 (2-(4-(2-((3-(Bis((Z)-2-hydroxyoctadec-9-en-1-yl)amino)propyl)disulfaneyl)ethyl)piperazin-1-yl)ethyl 4-(bis(2-hydroxydecyl)amino)butanoate), having a compound structure:

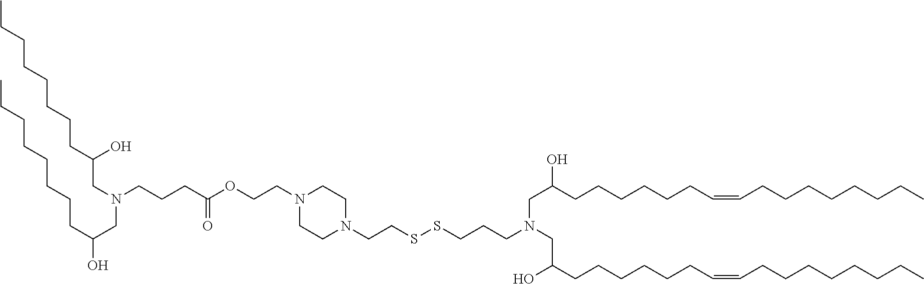 [SMILES: CCCCCCCC/C=C\CCCCCCC(O)CN(CCCSSCCN1CCN(CCOC(=O)CCCN(CC(O)CCCCCCCC)CC(O)CCCCCCCC)CC1)CC(O)CCCCCC/C=C\CCCCCCCC]

In some embodiments, the LNPs, compositions, pharmaceutical compositions and methods of the present invention include the cationic lipid GL-HEPES-E3-E12-DS-4-E10 (2-(4-(2-((3-(bis(2-hydroxydecyl)amino)butyl)disulfaneyl)ethyl)piperazin-1-yl)ethyl 4-(bis(2-hydroxydodecyl)amino)butanoate), having a compound structure:

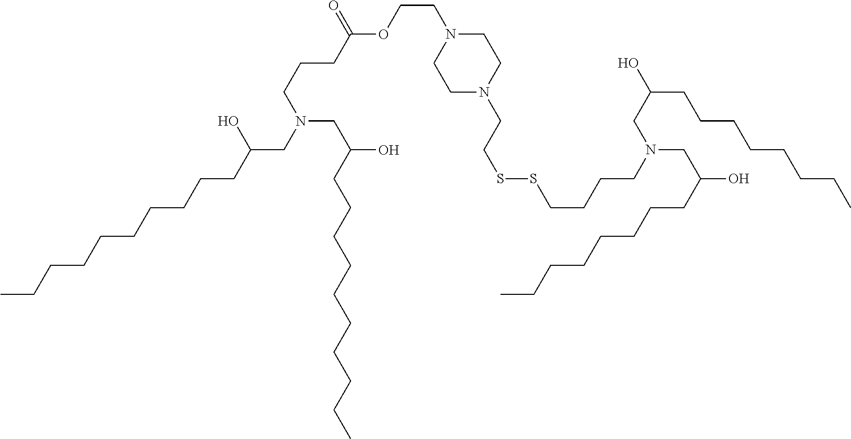 [SMILES: CCCCCCCCCCC(O)CN(CCCC(=O)OCCN1CCN(CCSSCCCCN(CC(O)CCCCCCCC)CC(O)CCCCCCCC)CC1)CC(O)CCCCCCCCCC]

In some embodiments, the LNPs, compositions, pharmaceutical compositions and methods of the present invention include the cationic lipid GL-HEPES-E3-E12-DS-3-E14 (2-(4-(2-((3-(Bis(2-hydroxytetradecyl)amino)propyl)disulfaneyl)ethyl)piperazin-1-yl)ethyl 4-(bis(2-hydroxydodecyl)amino)butanoate), having a compound structure:

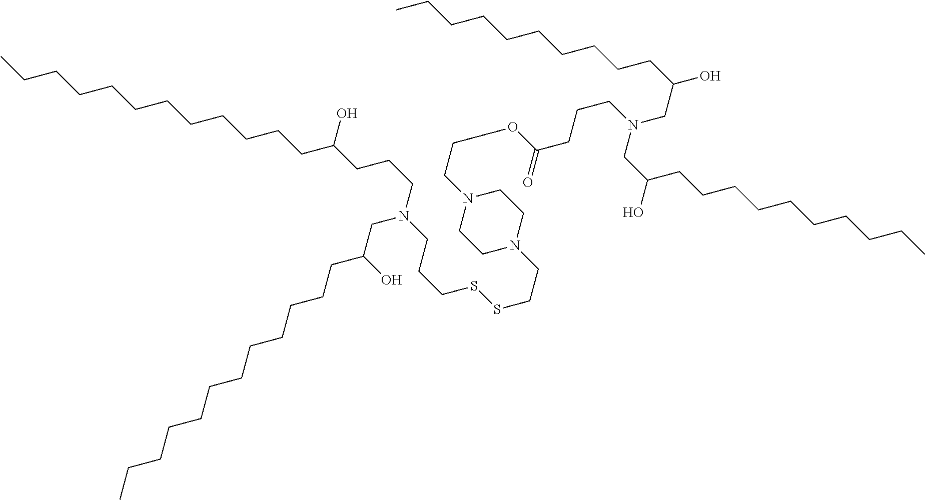 [SMILES: CCCCCCCCCCCCC(O)CCCN(CCCSSCCN1CCN(CCOC(=O)CCCN(CC(O)CCCCCCCCCC)CC(O)CCCCCCCCCC)CC1)CC(O)CCCCCCCCCCCC]

In some embodiments, the LNPs include the cationic lipid, N-[1-(2,3-dioleyloxy)propyl]-N,N,N-trimethylammonium chloride (“DOTMA”) (Feigner et al. (Proc. Nat'l Acad. Sci. 84, 7413 (1987); U.S. Pat. No. 4,897,355, which is incorporated herein by reference). Other cationic lipids suitable for the LNPs, compositions, pharmaceutical compositions and methods of the present invention include, for example, 5-carboxyspermylglycinedioctadecylamide (“DOGS”); 2,3-dioleyloxy-N-[2(spermine-carboxamido)ethyl]-N,N-dimethyl-1-propanaminium (“DOSPA”) (Behr et al. Proc. Nat.'l Acad. Sci. 86, 6982 (1989), U.S. Pat. Nos. 5,171,678; 5,334,761); 1,2-Dioleoyl-3-Dimethylammonium-Propane (“DODAP”); 1,2-Dioleoyl-3-Trimethylammonium-Propane (“DOTAP”).

In some embodiments, the LNPs include the cationic lipid IM-001 with the following structure:

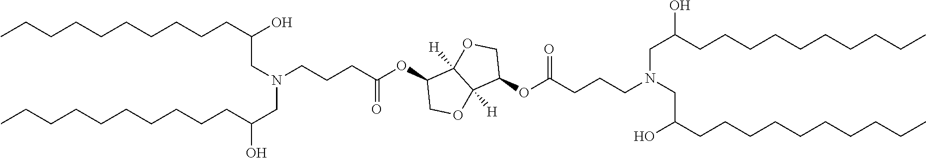 [SMILES: CCCCCCCCCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCCCCCC]

In some embodiments, the LNPs include the cationic lipid, N-[1-(2,3-dioleyloxy)propyl]-N,N,N-trimethylammonium chloride (“DOTMA”) (Feigner et al. (Proc. Nat'l Acad. Sci. 84, 7413 (1987); U.S. Pat. No. 4,897,355, which is incorporated herein by reference). Other cationic lipids suitable for the LNPs, compositions, pharmaceutical compositions and methods of the present invention include, for example, 5-carboxyspermylglycinedioctadecylamide (“DOGS”); 2,3-dioleyloxy-N-[2(spermine-carboxamido)ethyl]-N,N-dimethyl-1-propanaminium (“DOSPA”) (Behr et al. Proc. Nat.'l Acad. Sci. 86, 6982 (1989), U.S. Pat. Nos. 5,171,678; 5,334,761); 1,2-Dioleoyl-3-Dimethylammonium-Propane (“DODAP”); 1,2-Dioleoyl-3-Trimethylammonium-Propane (“DOTAP”).

, N-[1-(2,3-dioleyloxy)propyl]-N,N,N-trimethylammonium chloride (“DOTMA”) (Feigner et al. (Proc. Nat'l Acad. Sci. 84, 7413 (1987); U.S. Pat. No. 4,897,355, which is incorporated herein by reference). Other cationic lipids suitable for the LNPs, compositions, pharmaceutical compositions and methods of the present invention include, for example, 5-carboxyspermylglycinedioctadecylamide (“DOGS”); 2,3-dioleyloxy-N-[2(spermine-carboxamido)ethyl]-N,N-dimethyl-1-propanaminium (“DOSPA”) (Behr et al. Proc. Nat.'l Acad. Sci. 86, 6982 (1989), U.S. Pat. Nos. 5,171,678; 5,334,761); 1,2-Dioleoyl-3-Dimethylammonium-Propane (“DODAP”); 1,2-Dioleoyl-3-Trimethylammonium-Propane (“DOTAP”).

Additional exemplary cationic lipids suitable for the LNPs, compositions, pharmaceutical compositions and methods of the present invention also include: 1,2-distearyloxy-N,N-dimethyl-3-aminopropane (“DSDMA”); 1,2-dioleyloxy-N,N-dimethyl-3-aminopropane (“DODMA”); 1,2-dilinoleyloxy-N,N-dimethyl-3-aminopropane (“DLinDMA”); 1,2-dilinolenyloxy-N,N-dimethyl-3-aminopropane (“DLenDMA”); N-dioleyl-N,N-dimethylammonium chloride (“DODAC”); N,N-distearyl-N,N-dimethylammonium bromide (“DDAB”); N-(1,2-dimyristyloxyprop-3-yl)-N,N-dimethyl-N-hydroxyethyl ammonium bromide (“DMRIE”); 3-dimethylamino-2-(cholest-5-en-3-beta-oxybutan-4-oxy)-1-(cis,cis-9,12-octadecadienoxy)propane (“CLinDMA”); 2-[5′-(cholest-5-en-3-beta-oxy)-3′-oxapentoxy)-3-dimethy 1-1-(cis,cis-9′, 1-2′-octadecadienoxy)propane (“CpLinDMA”); N,N-dimethyl-3,4-dioleyloxybenzylamine (“DMOBA”); 1,2-N,N′-dioleylcarbamyl-3-dimethylaminopropane (“DOcarbDAP”); 2,3-Dilinoleoyloxy-N,N-dimethylpropylamine (“DLinDAP”); 1,2-N,N′-Dilinoleylcarbamyl-3-dimethylaminopropane (“DLincarbDAP”); 1,2-Dilinoleoylcarbamyl-3-dimethylaminopropane (“DLinCDAP”); 2,2-dilinoleyl-4-dimethylaminomethyl-[1,3]-dioxolane (“DLin-K-DMA”); 2-((8-[(3P)-cholest-5-en-3-yloxy]octyl)oxy)-N, N-dimethyl-3-[(9Z, 12Z)-octadeca-9, 12-dien-1-yloxy]propane-1-amine (“Octyl-CLinDMA”); (2R)-2-((8-[(3beta)-cholest-5-en-3-yloxy]octyl)oxy)-N, N-dimethyl-3-[(9Z, 12Z)-octadeca-9, 12-dien-1-yloxy]propan-1-amine (“Octyl-CLinDMA (2R)”); (2S)-2-((8-[(3P)-cholest-5-en-3-yloxy]octyl)oxy)-N, fsl-dimethyh3-[(9Z, 12Z)-octadeca-9, 12-dien-1-yloxy]propan-1-amine (“Octyl-CLinDMA (2S)”); 2,2-dilinoleyl-4-dimethylaminoethyl-[1,3]-dioxolane (“DLin-K-XTC2-DMA”); and 2-(2,2-di((9Z,12Z)-octadeca-9,12-dien-1-yl)-1,3-dioxolan-4-yl)-N,N-dimethylethanamine (“DLin-KC2-DMA”) (see, WO 2010/042877, which is incorporated herein by reference; Semple et al., Nature Biotech. 28: 172-176 (2010)). (Heyes, J., et al., J Controlled Release 107: 276-287 (2005); Morrissey, D V., et al., Nat. Biotechnol. 23(8): 1003-1007 (2005); International Patent Publication WO 2005/121348). In some embodiments, one or more of the cationic lipids comprise at least one of an imidazole, dialkylamino, or guanidinium moiety. In some embodiments, one or more cationic lipids suitable for the LNPs, compositions, pharmaceutical compositions and methods of the present invention include 2,2-Dilinoleyl-4-dimethylaminoethyl-[1,3]-dioxolane (“XTC”); (3aR,5s,6aS)-N,N-dimethyl-2,2-di((9Z,12Z)-octadeca-9,12-dienyl)tetrahydro-3aH-cyclopenta[d][1,3]dioxol-5-amine (“ALNY-100”) and/or 4,7,13-tris(3-oxo-3-(undecylamino)propyl)-N1,N16-diundecyl-4,7,10,13-tetraazahexadecane-1,16-diamide (“NC98-5”).

In a particular embodiment, the cationic lipid is selected from cKK-E12, cKK-E10, HGT5000, HGT5001, ICE, HGT4001, HGT4002, HGT4003, TL1-O1D-DMA, TL1-04D-DMA, TL1-08D-DMA, TL1-10D-DMA, OF-Deg-Lin, OF-02, GL-TES-SA-DMP-E18-2, GL-TES-SA-DME-E18-2, SY-3-E14-DMAPr, TL1-10D-DMA, HEP-E3-E10, HEP-E4-E10, RL3-DMA-07D, RL2-DMP-07D, cHse-E-3-E10, cHse-E-3-E12, cDD-TE-4-E12, SI-4-E14-DMAPr, TL-1-12D-DMA, SY-010, SY-011, GL-HEPES-E3-E12-DS-4-E10, and 4-hydroxybutyl)azanediyl)bis(hexane-6,1-diyl)bis(2-hexyldecanoate (ALC-0315);

In a specific embodiment, the cationic lipid is selected from cKKE10, OF-02, GL-HEPES-E3-E12-DS-4-E10, and ALC-0315.

In some embodiments, the LNPs, compositions, pharmaceutical compositions of the present invention include one or more cationic lipids that constitute at least about 5%, 10%, 20%, 30%, 35%, 40%, 45%, 50%, 55%, 60%, 65%, or 70%, measured by weight, of the total lipid content in the LNPs, compositions, pharmaceutical composition, e.g., a lipid nanoparticle. In some embodiments, the LNPs, compositions, pharmaceutical compositions of the present invention include one or more cationic lipids that constitute at least about 5%, 10%, 20%, 30%, 35%, 40%, 45%, 50%, 55%, 60%, 65%, or 70%, measured as a mol %, of the total lipid content in the LNPs, compositions, pharmaceutical composition, e.g., a lipid nanoparticle. In some embodiments, the LNPs, compositions, pharmaceutical compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured by weight, of the total lipid content in the LNPs, compositions, pharmaceutical composition, e.g., a lipid nanoparticle. In some embodiments, the LNPs, compositions, pharmaceutical compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured as mol %, of the total lipid content in the LNPs, compositions, pharmaceutical composition, e.g., a lipid nanoparticle.

### Non-Cationic Lipids

In some embodiments, the lipid nanoparticles contain one or more non-cationic lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. The non-cationic may enhance the structural stability of the LNP and improve uptake and release of the mRNA payload. In some embodiments, the non-cationic lipid is a zwitterionic lipid, which has fusogenic properties for enhancing uptake and release of the mRNA payload. Non-cationic lipids include, but are not limited to, distearoylphosphatidylcholine (DSPC), dioleoylphosphatidylcholine (DOPC), dipalmitoylphosphatidylcholine (DPPC), 1,2-dilauroyl-sn-glycero-3-phosphocholine (DLPC), dioleoylphosphatidylglycerol (DOPG), dipalmitoylphosphatidylglycerol (DPPG), dioleoylphosphatidylethanolamine (DOPE), 1,2-dioleoyl-sn-glycero-3-phospho-L-serine (DOPS), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 1,2-dilauroyl-sn-glycero-3-phosphoethanolamine (DLPE), 1,2-dierucoyl-sn-glycero-3-phosphoethanolamine (DEPE), 1,2-dioleoyl-sn-glycero-3-phosphocholine (DPOC), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a mixture thereof. In some embodiments, lipid nanoparticles suitable for use with the invention include DOPE as the non-cationic lipid component. In other embodiments, lipid nanoparticles suitable for use with the invention include DEPE as the non-cationic lipid component.

In a particular embodiment, the non-cationic lipid is selected from DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DEPE 1,2-dierucoyl-sn-glycero-3-phosphoethanolamine, DOPC (1,2-dioleyl-sn-glycero-3-phosphotidylcholine), DPPE (1,2-dipalmitoyl-sn-glycero-3-phosphoethanolamine), DMPE (1,2-dimyristoyl-sn-glycero-3-phosphoethanolamine), and DOPG (1,2-dioleoyl-sn-glycero-3-phospho-(l′-rac-glycerol)). In a specific embodiment, the non-cationic lipid is selected from DOPE and/or DSPC.

In some embodiments, a non-cationic lipid is a neutral lipid, i.e., a lipid that does not carry a net charge in the conditions under which the LNPs, compositions, pharmaceutical compositions are formulated and/or administered.

### Cholesterol-Based Lipids

In some embodiments, the lipid nanoparticle comprises one or more cholesterol-based lipids. The cholesterol-based lipid may provide stability to the lipid bilayer structure within the nanoparticle. For example, suitable cholesterol-based cationic lipids include, for example, DC-Chol (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), orimidazole cholesterol ester (ICE), as disclosed in International Patent Publication WO 2011/068810, which has the following structure:

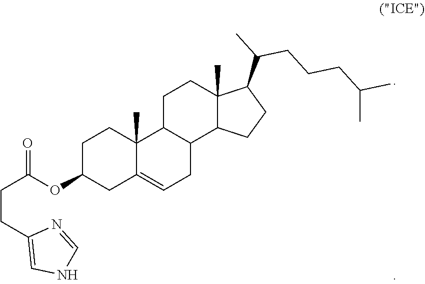 [SMILES: CC(C)CCCC(C)[C@H]1CCC2C3CC=C4C[C@@H](OC(=O)CCc5c[nH]cn5)CC[C@]4(C)C3CC[C@@]21C]

In embodiments, a cholesterol-based lipid is cholesterol.

### PEG-Modified Lipids

In some embodiments, the lipid nanoparticle comprises one or more PEGylated lipids. The PEGylated lipid may provide control over particle size and stability of the LNP. The addition of such components may prevent complex aggregation and provide a means for increasing circulation lifetime and increasing the delivery of the lipid-nucleic acid pharmaceutical composition to target tissues (Klibanov et al., FEBS Letters (1990) 268 (1):235-7). These components may be selected to rapidly exchange out of the pharmaceutical composition in vivo (see, e.g., U.S. Pat. No. 5,885,613).

For example, the use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000](C₈ PEG-2000 ceramide) is also contemplated by the present invention, either alone or preferably in combination with other lipid pharmaceutical compositions together which comprise the transfer vehicle (e.g., a lipid nanoparticle).

Contemplated PEG-modified lipids include, but are not limited to, a polyethylene glycol chain of up to 5 kDa in length covalently attached to a lipid with alkyl chain(s) of C₆-C₂₀ (e.g., C₈, C₁₀, C₁₂, C₁₄, C₁₆, or Cis) length. In some embodiments, a PEG-modified or PEGylated lipid is PEGylated cholesterol or PEG-2K. The addition of such components may prevent complex aggregation and may also provide a means for increasing circulation lifetime and increasing the delivery of the lipid-nucleic acid pharmaceutical composition to the target tissues, (Klibanov et al. (1990) FEBS Letters, 268 (1): 235-237), or they may be selected to rapidly exchange out of the pharmaceutical composition in vivo (see U.S. Pat. No. 5,885,613). Particularly useful exchangeable lipids are PEG-ceramides having shorter acyl chains (e.g., C₁₄ or C₁₈).

In some embodiments, the PEGylated lipid is 1,2-dimyristoyl-rac-glycero-3-methoxypolyethylene glycol (DMG-PEG); 1,2-distearoyl-sn-glycero-3-phosphoethanolamine-polyethylene glycol (DSPE-PEG); 1,2-dilauroyl-sn-glycero-3-phosphoethanolamine-polyethylene glycol (DLPE-PEG); or 1,2-distearoyl-rac-glycero-polyethelene glycol (DSG-PEG).

The PEG typically has a high molecular weight, e.g., 2000-2400 g/mol. In some embodiments, the PEG is PEG2000 (or PEG-2K). In particular embodiments, the PEGylated lipid is DMG-PEG2000, DSPE-PEG2000, DLPE-PEG2000, DSG-PEG2000, or C₈ PEG2000.

LNPs suitable for use with the invention typically include a PEG-modified lipid such as 1,2-dimyristoyl-rac-glycero-3-methoxypolyethylene glycol-2000 (DMG-PEG2K) or 2-[(polyethylene glycol)-2000]-N,N-ditetradecylacetamide (ALC-0159).

In some embodiments, one or more PEG-modified lipids constitute about 4% of the total lipids by molar ratio. In some embodiments, one or more PEG-modified lipids constitute about 5% of the total lipids by molar ratio. In some embodiments, one or more PEG-modified lipids constitute about 6% of the total lipids by molar ratio. For certain applications, such as pulmonary delivery, lipid nanoparticles in which the PEG-modified lipid component constitutes about 5% of the total lipids by molar ratio have been found to be particularly suitable.

### Exemplary Lipid Formulations

A typical LNP for use with the invention may be composed of one of the following combinations of a cationic lipid, a non-cationic lipid, a PEG-modified lipid and optionally cholesterol: cKK-E12, DOPE, cholesterol and DMG-PEG2K; cKK-E10, DOPE, cholesterol and DMG-PEG2K; OF-Deg-Lin, DOPE, cholesterol and DMG-PEG2K; OF-02, DOPE, cholesterol and DMG-PEG2K; GL-HEPES-E3-E12-DS-4-E10, DOPE, cholesterol and DMG-PEG2K; C12-200, DOPE, cholesterol and DMG-PEG2K; HGT4003, DOPE, cholesterol and DMG-PEG2K; ICE, DOPE, cholesterol and DMG-PEG2K; HGT4001, DOPE, cholesterol and DMG-PEG2K; HGT4002, DOPE, cholesterol and DMG-PEG2K; TL1-01D-DMA, DOPE, cholesterol and DMG-PEG2K; TL1-04D-DMA, DOPE, cholesterol and DMG-PEG2K; TL1-08D-DMA, DOPE, cholesterol and DMG-PEG2K; TL1-10D-DMA, DOPE, cholesterol and DMG-PEG2K; ICE, DOPE and DMG-PEG2K; HGT4001, DOPE and DMG-PEG2K; HGT4002, DOPE and DMG-PEG2K; SY-3-E14-DMAPr, DOPE, cholesterol and DMG-PEG2K; RL3-DMA-07D, DOPE, cholesterol and DMG-PEG2K; RL2-DMP-07D, DOPE, cholesterol and DMG-PEG2K; cHse-E-3-E10, DOPE, cholesterol and DMG-PEG2K; cHse-E-3-E12, DOPE, cholesterol and DMG-PEG2K; or cDD-TE-4-E12, DOPE, cholesterol and DMG-PEG2K. In specific embodiments, the LNP may be composed of SY-3-E14-DMAPr, DOPE, cholesterol and DMG-PEG2K. In other specific embodiments, the LNP may be composed of RL3-DMA-07D, DOPE, cholesterol and DMG-PEG2K. In yet other specific embodiments, the LNP may be composed of RL2-DMP-07D, DOPE, cholesterol and DMG-PEG2K. In yet other specific embodiments, the LNP may be composed of cHse-E-3-E10, DOPE, cholesterol and DMG-PEG2K. In yet other specific embodiments, the LNP may be composed of cHse-E-3-E12, DOPE, cholesterol and DMG-PEG2K. In yet other specific embodiments, the LNP may be composed of cDD-TE-4-E12, DOPE, cholesterol and DMG-PEG2K.

The molar ratio of the cationic lipid, the PEGylated lipid, the cholesterol-based lipid, and the non-cationic lipid is A:B:C:D, where A+B+C+D=100%. In some embodiments, the molar ratio of the cationic lipid in the LNPs relative to the total lipids (i.e., A) is 35-45% (e.g., 38-42% such as 40%). In some embodiments, the molar ratio of the PEGylated lipid component relative to the total lipids (i.e., B) is 0.25-2.75% (e.g., 1-2% such as 1.5%). In some embodiments, the molar ratio of the cholesterol-based lipid relative to the total lipids (i.e., C) is 20-35% (e.g., 27-30% such as 28.5%). In some embodiments, the molar ratio of the non-cationic lipid relative to the total lipids (i.e., D) is 25-35% (e.g., 28-32% such as 30%). In some embodiments, the (PEGylated lipid+cholesterol) components have the same molar amount as the helper lipid. In some embodiments, the LNPs contain a molar ratio of the cationic lipid to the helper lipid that is more than 1.

In some embodiments, cationic lipids (e.g., cKK-E12, cKK-E10, OF-Deg-Lin, OF-02, GL-HEPES-E3-E12-DS-4-E10, TL1-01D-DMA, TL1-04D-DMA, TL1-08D-DMA, TL1-10D-DMA, ICE, HGT4001, and/or HGT4002) constitute about 30-60% (e.g., about 30-55%, about 30-50%, about 30-45%, about 30-40%, about 35-50%, about 35-45%, or about 35-40%) of the lipid nanoparticle by molar ratio. In some embodiments, the percentage of cationic lipids (e.g., cKK-E12, cKK-E10, OF-Deg-Lin, OF-02, GL-HEPES-E3-E12-DS-4-E10, TL1-01D-DMA, TL1-04D-DMA, TL1-08D-DMA, TL1-10D-DMA, ICE, HGT4001, and/or HGT4002) is or greater than about 30%, about 35%, about 40%, about 45%, about 50%, about 55%, or about 60% of the lipid nanoparticle by molar ratio.

In some embodiments, the ratio of cationic lipid(s) to non-cationic lipid(s) to cholesterol-based lipid(s) to PEG-modified lipid(s) may be between about 30-60:25-35:20-30:1-15 by molar ratio. In some embodiments, the ratio of cationic lipid(s) to non-cationic lipid(s) to cholesterol-based lipid(s) to PEG-modified lipid(s) is approximately 40:30:20:10 by molar ratio. In some embodiments, the ratio of cationic lipid(s) to non-cationic lipid(s) to cholesterol-based lipid(s) to PEG-modified lipid(s) is approximately 40:30:25:5 by molar ratio. In some embodiments, the ratio of cationic lipid(s) to non-cationic lipid(s) to cholesterol-based lipid(s) to PEG-modified lipid(s) is approximately 40:32:25:3 by molar ratio. In some embodiments, the ratio of cationic lipid(s) to non-cationic lipid(s) to cholesterol-based lipid(s) to PEG-modified lipid(s) is approximately 50:25:20:5 by molar ratio.

In certain embodiments, the LNP comprises: a cationic lipid (e.g., OF-02, cKK-E10, or GL-HEPES-E3-E12-DS-4-E10) at a molar ratio of 35% to 55%; a non-cationic lipid (e.g., DOPE) at a molar ratio of 5% to 40%; a cholesterol-based lipid (e.g., cholesterol) at a molar ratio of 20% to 45%; and a PEG-modified lipid (e.g., DMG-PEG2K) at a molar ratio of 1% to 2%.

In some embodiments, the molar ratio of cationic lipid to non-cationic lipid to cholesterol-based lipid to PEG-modified lipid of 40:30:28.5:1.5. In some embodiments, the molar ratio of cationic lipid to non-cationic lipid to cholesterol-based lipid to PEG-modified lipid of 46.3:9.4:42.7:1.6. In some embodiments, the molar ratio of cationic lipid to non-cationic lipid to cholesterol-based lipid to PEG-modified lipid of 50:10:38.5:1.5.

In some embodiments, the LNP comprises: OF-02, c-KK-E10, or GL-HEPES-E3-E12-DS-4-E10 at a molar ratio of 40%; DOPE at a molar ratio of 30%; cholesterol at a molar ratio of 28.5%; and DMG-PEG2K at a molar ratio of 1.5%. In some embodiments, the LNP comprises: ALC-0315 at a molar ratio of 46.3%; DSPC at a molar ratio of 9.4%; cholesterol at a molar ratio of 42.7%; and ALC-0159 at a molar ratio of 1.6%. In some embodiments, the LNP comprises: SM-102 at a molar ratio of 50%; DSPC at a molar ratio of 10%; cholesterol at a molar ratio of 38.5%; and DMG-PEG2K at a molar ratio of 1.5%. Such lipid nanoparticles are particularly suitable for the delivery of mRNA via intramuscular administration.

In typical three-component lipid nanoparticles suitable for use with the invention, the molar ratio of cationic lipid to non-cationic lipid to PEG-modified lipid may be between about 55-65:30-40:1-15, respectively. In some embodiments, a molar ratio of cationic lipid (e.g., a sterol-based lipid) to non-cationic lipid (e.g., DOPE or DEPE) to PEG-modified lipid (e.g., DMG-PEG2K) of 60:35:5 is particularly suitable, e.g., for pulmonary delivery of lipid nanoparticles via nebulization.

### One or More LNPs

In some embodiments, an LNP may carry mRNAs that encode more than one protein, such as two, three, four, five, six, seven, eight, nine, ten, or more proteins. For example, an LNP may carry a polycistronic mRNA (e.g., a bicistronic mRNA) that can be translated into more than one influenza virus protein (e.g., each protein-coding sequence is separated by a nucleotide sequence encoding an IRES or a self-cleaving peptide such as a 2A peptide). Typically, a polycistronic mRNA encodes no more than three or four proteins.

In some embodiments, one mRNA encodes an HA protein and an NA protein. For example, the one or more HA proteins and the one or more NA proteins may be encoded by the same mRNA. In a specific embodiment, the mRNA encoding for both HA and NA proteins is separate to the mRNA encoding the M1 protein.

In some embodiments, one mRNA encodes one or more HA proteins and one or more NA proteins from an influenza A strain. In some embodiments, the encoded HA and NA proteins of influenza A are encoded by the same mRNA. In some embodiments, the mRNA encodes one or more HA proteins and one or more NA proteins from influenza B strain. In some embodiments, the encoded HA and NA proteins of influenza B are encoded by the same mRNA. In one embodiment, the one or more HA proteins and the one or more NA proteins are from a combination of influenza A and B strains and are encoded from the same mRNA.

In some embodiments, an LNP comprises a first mRNA encoding a first HA protein and a first NA protein, a second mRNA encoding a second HA protein and a second NA protein, and a third mRNA encoding an M1 protein. Typically, the first HA protein and the first NA protein are from a first influenza virus (e.g., H1 and N1 from influenza A), and the second HA protein and the second NA protein are from a second influenza virus (e.g., H3 and N2 from influenza A). In some embodiments, the first and second influenza viruses are influenza A viruses of different subtypes. In some embodiments, the first and second viruses are influenza B viruses of different lineages.

In some embodiments, the LNP further comprises a fourth mRNA encoding a third HA protein and a third NA protein from a third influenza virus, a fifth mRNA encoding a fourth HA protein and a fourth NA protein from a fourth influenza virus. The third influenza virus typically is an influenza virus B virus of the Yamagata lineage and the fourth influenza virus is of the Victoria lineage.

In some embodiments, an LNP comprises a first mRNA encoding at least a first HA protein and a second HA protein, a second mRNA encoding at least a first NA protein and a second NA protein, and a third mRNA encoding an M1 protein. Typically, the first HA protein and the second HA protein (and any further HA proteins) are from different subtypes (e.g., H1 and H3 from influenza A) and/or lineages, and the first NA protein and the second NA protein (and any further NA proteins) are from different subtypes (e.g., N1 and N2 from influenza A) and/or lineages.

In some embodiments, the LNP comprises a fourth mRNA encoding a third HA protein and a third NA protein from a third influenza virus. In some embodiments, the first influenza virus is an H1N1 influenza A virus, the second influenza virus is an H3N2 influenza virus and the third influenza virus is an influenza B virus (e.g., influenza B/Yamagata or influenza B/Victoria).

In some embodiments, the LNP comprises a fifth mRNA encoding a fourth HA protein and a fourth NA protein from a fourth influenza virus. In some embodiments, the fourth influenza virus is an influenza B virus from a lineage different to that of the third influenza virus.

In some embodiments, the first, second, and third mRNAs are encapsulated in a first LNP and the fourth and fifth mRNA are encapsulated in a second LNP. The second LNP may additionally comprise a sixth mRNA. In some embodiments, the sixth mRNA encodes an M1 protein. In some embodiments, the M1 protein encoded by the sixth mRNA is from an influenza B virus.

In some embodiments, the M1, HA, and NA proteins are each encoded by separate mRNAs and are all mRNAs encapsulated in a single LNP. For example, an LNP may comprises three, four, five, six, seven, eight, nine, or more mRNA molecules encoding (i) two or more HA proteins, and/or (ii) two or more NA proteins, or (iii) at least one HA protein and at least one NA protein. In some embodiments, an LNP comprises two mRNAs encoding HA proteins (e.g., H1 and H3), two mRNAs encoding NA proteins (e.g., N1 and N2), and one mRNA encoding an M1 protein. In some embodiments, an LNP comprises three mRNAs encoding HA proteins (e.g., H1, H3, and influenza B HA), three mRNAs encoding NA proteins (e.g., (N1, N2, and an influenza B HA), and one mRNA encoding an M1 protein. In some embodiments, an LNP comprises four mRNAs encoding HA proteins (e.g., two influenza A and two influenza B HAs), four mRNAs encoding NA proteins (e.g., two influenza A and two influenza B HAs), and one mRNA encoding an M1 protein.

In some embodiments, an LNP comprises at least one mRNA encoding an influenza type A HA protein (e.g., H1 or H3), at least one mRNA encoding an influenza type A NA protein (e.g., N1 or N2), and one mRNA encoding an M1 protein, wherein the HA mRNA, the NA mRNA and the M1 mRNA are present at molar ratios of 40-50:10-50:30-50 (e.g., 40:10:50, 40:40:50, or 50:10:30). In some embodiments, an LNP comprises at least one mRNA encoding an influenza type B HA protein (e.g., Yamagata or Victoria), at least one mRNA encoding influenza type B NA protein (e.g., Yamagata or Victoria), and one mRNA encoding an M1 protein, wherein the HA mRNA, the NA mRNA and the M1 mRNA are present at molar ratios of 40-50:10-50:30-50 (e.g., 40:10:50, 40:40:50, or 50:10:30). In some embodiments, a composition described herein comprises a first LNP comprising at least one mRNA encoding an influenza type A HA protein (e.g., H1 or H3), at least one mRNA encoding influenza type A NA protein (e.g., N1 or N2), and one mRNA encoding an M1 protein, wherein the HA mRNA, the NA mRNA and the M1 mRNA are present at molar ratios of 40-50:10-50:30-50 (e.g., 40:10:50, 40:40:50, or 50:10:30), and a second LNP comprising at least one mRNA encoding an influenza type B HA protein (e.g., Yamagata or Victoria), at least one mRNA encoding influenza type B NA protein (e.g., Yamagata or Victoria) and one mRNA encoding an M1 protein, wherein the HA mRNA, the NA mRNA and the M1 mRNA are present at molar ratios of 40-50:10-50:30-50 (e.g., 40:10:50, 40:40:50, or 50:10:30).

In some embodiments, an LNP comprises two mRNAs encoding influenza A HA proteins (e.g., H1 and H3), two mRNAs encoding influenza A NA proteins (e.g., (N1 and N2), and one mRNA encoding an M1 protein, wherein the HA mRNAs and the NA mRNAs are present at a molar ratio of 40:10 or 50:10, or at equal molar ratios. In some embodiments, an LNP comprises two mRNAs encoding influenza B HA proteins (e.g., H1 and H3), two mRNAs encoding influenza B NA proteins (e.g., (N1 and N2), and one mRNA encoding an M1 protein, wherein the HA mRNAs and the NA mRNAs are present at a molar ratio of 40:10 or 50:10, or at equal molar ratios.

In some embodiments, the M1, HA, and NA proteins are each encoded by separate mRNAs and each mRNA is separately encapsulated in an LNP.

In some embodiments, sets of mRNAs deriving from the same influenza virus may be formulated in a single LNP. For example, a first set of mRNAs encoding HA and NA proteins from a first influenza A virus may be encapsulated in a first LNP. A second set of mRNAs encoding HA and NA proteins from a second influenza A virus may be encapsulated in a second LNP. A third set of mRNAs encoding HA and NA proteins from an influenza B virus may be encapsulated in a third LNP. Each set of mRNAs may comprise an mRNA encoding an HA protein, an mRNA encoding an NA protein and an mRNA encoding an M1 protein. The M1 protein may be the same for each set. In some embodiments, the HA and NA protein may be encoded by the same mRNA. The first, second and third LNPs each may have the same lipid composition. In some embodiments, the first, second and third LNPs have different lipid compositions.

In some embodiments, a fourth set of mRNAs encoding HA and NA proteins from an influenza B virus of a different lineage from that of the HA and NA proteins encoded by the mRNA in the third set are encapsulated in a fourth LNP. The first, second, third and fourth LNPs each may have the same lipid composition. In some embodiments, the first, second, third and fourth LNPs have different lipid compositions.

In some circumstances, it may be convenient to encapsulate mRNAs encoding HA and NA proteins from first and second influenza A viruses in a first LNP, and the mRNAs encoding HA and NA proteins from the one or more influenza B viruses in a second LNP. Accordingly, in some embodiments, the first and second sets of mRNAs are encapsulated in a first LNP, and the third and optionally fourth sets of mRNAs are encapsulated in second LNP. The first and second LNPs may have the same lipid composition. In some embodiments, the first and second LNPs have different lipid compositions.

### Polymers

In some embodiments, a suitable LNP delivery vehicle is formulated using a polymer as a carrier, alone, or in combination with other carriers including various lipids described herein. Thus, in some embodiments, LNPs, as used herein, also encompass nanoparticles comprising polymers. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PLL, PEGylated PLL and polyethylenimine (PEI). When PEI is present, it may be branched PEI of a molecular weight ranging from 10 to 40 kDa, e.g., 25 kDa branched PEI (Sigma #408727).

### Compositions

In some embodiments, a composition (e.g., an immunogenic composition or vaccine) in accordance with the invention comprises mRNA at a concentration ranging from about 0.5 mg/mL to about 1.0 mg/mL. In some embodiments, mRNA is at a concentration of at least 0.5 mg/mL. In some embodiments, mRNA is at a concentration of at least 0.6 mg/mL. In some embodiments, mRNA is at a concentration of at least 0.7 mg/mL. In some embodiments, mRNA is at a concentration of at least 0.8 mg/mL. In some embodiments, mRNA is at a concentration of at least 0.9 mg/mL. In some embodiments, mRNA is at a concentration of at least 1.0 mg/mL. In a typical embodiment, mRNA is at a concentration of about 0.6 mg/mL to about 0.8 mg/mL.

Typically, the mRNA in the composition (e.g., an immunogenic composition or vaccine) is encapsulated in LNPs. To stabilize the mRNA or the LNPs encapsulating it, or to enhance in vivo expression of the mRNAs, the compositions of the invention may be formulated with one or more carrier, stabilizing reagent or other excipients. Such compositions may be pharmaceutical compositions, and as such they may include one more or more pharmaceutically acceptable excipients. The one or more pharmaceutically acceptable excipients may be selected from a buffer, a sugar, a salt, a surfactant or combinations thereof.

### Pharmaceutically Acceptable Excipients

In some embodiments, the pharmaceutical composition (e.g., an immunogenic composition or vaccine) is formulated with a diluent. In some embodiments, the diluent is selected from a group consisting of ethylene glycol, glycerol, propylene glycol, sucrose, trehalose, or combinations thereof. In some embodiments, the diluent is a disaccharide (e.g., trehalose or sucrose). In some embodiments, the formulation comprises 1%, 2%, 3%, 4%, 5%, 6%, 7%, 8%, 9%, 10%, 11%, 12%, 13%, 14%, 15%, 16%, 17%, 18%, 19% or 20% diluent.

In some embodiments, the LNPs are suspended in an aqueous solution comprising a disaccharide. Suitable disaccharides for use with the invention include trehalose and sucrose. For example, in some embodiments, the LNPs are suspended in an aqueous solution comprising trehalose, e.g., 10% (w/v) trehalose in water. In other embodiments, LNPs are suspended in an aqueous solution comprising sucrose, e.g., 10% (w/v) sucrose in water.

In some embodiments, the aqueous solution further comprises a buffer, a salt, a surfactant, or combinations thereof.

In some embodiments, the salt is selected from the group consisting of NaCl, KCl, and CaCl₂. In some embodiments, the salt is NaCl. In some embodiments, the salt is KCl. In some embodiments, the salt is CaCl₂. In some embodiments, the salt is a combination of KCl and NaCl.

In some embodiments, the buffer is selected from the group consisting of a phosphate buffer, a citrate buffer, an imidazole buffer, a histidine buffer, and a Good's buffer. Accordingly, in some embodiments, the buffer is a phosphate buffer. In some embodiments, the buffer is a citrate buffer. In some embodiments, the buffer is an imidazole buffer. In some embodiments, the buffer is a histidine buffer. In some embodiments, the buffer is a Good's buffer. In some embodiments, the Good's buffer is a Tris buffer or HEPES buffer.

In particular embodiments, the buffer is a phosphate buffer (e.g., a citrate-phosphate buffer), a Tris buffer (e.g., TrisHCl), or an imidazole buffer. In some embodiments, the buffer is or includes an acetate buffer.

In some embodiments, the composition (e.g., the immunogenic composition or vaccine) comprises a buffer and a salt (typically in addition to a suitable diluent such as a disaccharide or optionally a propylene glycol). In some embodiments, the total concentration of the buffer and the salt is selected from about 40 mM Tris buffer and about 75-125 mM NaCl, about 50 mM Tris buffer and about 50 mM-100 mM NaCl, about 100 mM Tris buffer and about 100 mM-200 mM NaCl, about 40 mM imidazole and about 100 mM-125 mM NaCl, and about 50 mM imidazole and 75 mM-100 mM NaCl.

In some embodiments, the composition (e.g., the immunogenic composition or vaccine) comprises a buffer (e.g., phosphate or Tris), a salt (e.g., KCl or NaCl, or both), and a sugar (e.g., a disaccharide such as sucrose or trehalose). In particular embodiments, the composition (e.g., the immunogenic composition or vaccine) is an aqueous solution (e.g., comprising water for injection) comprising the buffer, salt and sugar. Additional excipients may include NaOH or HCl (e.g., to adjust the pH of the composition).

### Lipid Nanoparticle Formulations

In some embodiments, the majority of LNPs in a composition of the invention (e.g., an immunogenic composition or vaccine of the invention), i.e., greater than about 50%, 55%, 60%, 65%, 70%, 75%, 80%, 85%, 90%, 95%, 96%, 97%, 98%, or 99% of the LNPs, have a size of about 150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, the LNPs in a composition of the invention have a size of about 150 nm or less (e.g., about 145 nm or less, about 140 nm or less, about 135 nm or less, about 130 nm or less, about 125 nm or less, about 120 nm or less, about 115 nm or less, about 110 nm or less, about 105 nm or less, about 100 nm or less, about 95 nm or less, about 90 nm or less, about 85 nm or less, or about 80 nm or less). In specific embodiments, the LNPs are between 70 nm and about 150 nm in size.

In some embodiments, greater than about 70%, 75%, 80%, 85%, 90%, 95%, 96%, 97%, 98%, 99% of the LNPs in a composition (e.g., an immunogenic composition or vaccine) provided by the present invention have a size ranging from about 40-90 nm (e.g., about 45-85 nm, about 50-80 nm, about 55-75 nm, about 60-70 nm). In some embodiments, the LNPs have a size ranging from about 40-90 nm (e.g., about 45-85 nm, about 50-80 nm, about 55-75 nm, about 60-70 nm). Compositions with LNPs having an average size of about 50-70 nm (e.g., 55-65 nm) may be particularly suitable for delivery via nebulization or inhalation.

In some embodiments, the dispersity, or measure of heterogeneity in size of molecules (polydispersity index; PDI), of lipid nanoparticles in a pharmaceutical composition provided by the present invention is less than about 0.5. In some embodiments, a lipid nanoparticle has a PDI of less than about 0.5. In some embodiments, a lipid nanoparticle has a PDI of less than about 0.4. In some embodiments, a lipid nanoparticle has a PDI of less than about 0.3. In some embodiments, a lipid nanoparticle has a PDI of less than about 0.28. In some embodiments, a lipid nanoparticle has a PDI of less than about 0.25. In some embodiments, a lipid nanoparticle has a PDI of less than about 0.23. In some embodiments, a lipid nanoparticle has a PDI of less than about 0.20. In some embodiments, a lipid nanoparticle has a PDI of less than about 0.18. In some embodiments, a lipid nanoparticle has a PDI of less than about 0.16. In some embodiments, a lipid nanoparticle has a PDI of less than about 0.14. In some embodiments, a lipid nanoparticle has a PDI of less than about 0.12. In some embodiments, a lipid nanoparticle has a PDI of less than about 0.10. In some embodiments, a lipid nanoparticle has a PDI of less than about 0.08.

In some embodiments, an LNP has an mRNA encapsulation efficiency of greater than about 80%. In some embodiments, an LNP has an mRNA encapsulation efficiency of greater than about 85%. In some embodiments, an LNP has an mRNA encapsulation efficiency of greater than about 90%. In some embodiments, an LNP has an mRNA encapsulation efficiency of greater than about 92%. In some embodiments, an LNP has an mRNA encapsulation efficiency of greater than about 95%. In some embodiments, an LNP has an mRNA encapsulation efficiency of greater than about 98%. In some embodiments, an LNP has an mRNA encapsulation efficiency of greater than about 99%. Typically, LNPs for use with the invention have an mRNA encapsulation efficiency of at least 90%-95%.

### Therapeutically Effective Amount

The mRNA in accordance with the invention is provided in a therapeutically effective amount in the pharmaceutical compositions, the immunogenic composition or vaccine provided herein. As used herein, the term “therapeutically effective amount” is largely determined based on the total amount of the therapeutic agent contained in the pharmaceutical compositions of the present invention. Generally, a therapeutically effective amount is sufficient to achieve a meaningful benefit to the subject.

In some embodiments, a single-vial dose contains 1-50 g of mRNA (e.g., monovalent or multivalent). For example, a single-dose vial or syringe may contain about 2.5 g, about 5 g, about 7.5 g, about 10 g, about 12.5 g, or about 15 g of the mRNA, typically to be administered by intramuscular injection or by mucosal delivery.

### Packaging

The compositions of the invention (e.g., the immunogenic compositions or vaccines of the invention) may be packaged for parenteral (e.g., intramuscular, intradermal or subcutaneous) administration or mucosal (e.g., nasopharyngeal, pulmonary or intranasal) administration. The vaccine compositions may be in the form of an extemporaneous formulation, e.g., in a lyophilized form that requires reconstitution with a physiological buffer (e.g., PBS) just before use. In some embodiments, a composition of the invention (e.g., an immunogenic composition or vaccine of the invention) is provided in the form of an aqueous solution or a frozen aqueous solution and can be directly administered to subjects without reconstitution (after thawing, if previously frozen).

In some embodiments, a composition of the invention comprises separate mRNAs, e.g., one mRNA encoding an HA protein, one mRNA encoding an NA protein, and one mRNA encoding an M1 protein. In some embodiments, the HA mRNA, the NA mRNA and the M1 mRNA are provided at a weight ratio of 1:1:2. In some embodiments, the HA mRNA, the NA mRNA, and the M1 mRNA are encapsulated in the same lipid nanoparticle.

In some embodiments, the composition is multivalent, e.g., comprises multiple sets each comprising an HA mRNA, an NA mRNA, and an M1 mRNA from a different influenza virus. For example, a quadrivalent composition may comprise four sets of mRNAs, for instance a first set of mRNAs from an influenza A virus (H1N1), a second set of mRNAs from an influenza A virus (H3N2), a third set of mRNAs from an influenza B virus (B/Yamagata), and a fourth set from an influenza B virus (B/Victoria). Each set of mRNAs may be encapsulated in the same lipid nanoparticle. In some embodiments, the HA mRNA, the NA mRNA and the M1 mRNA in each set are provided at a weight ratio of 1:1:2.

Accordingly, the present disclosure provides an article of manufacture, such as a kit, that provides the composition of the invention (e.g., the immunogenic composition or vaccine) in a single container, or provides the composition (e.g., the immunogenic composition or vaccine) in one container and a physiological buffer for reconstitution in another container. The container(s) may contain a single-use dosage or multi-use dosage. The containers may be pre-treated glass vials or ampules. The article of manufacture may include instructions for use as well.

In particular embodiments, a composition of the invention (e.g., an immunogenic composition or vaccine of the invention) is provided for use in intramuscular injection. The composition can be injected to a subject at, e.g., his/her deltoid muscle in the upper arm. In some embodiments, the composition (e.g., the immunogenic composition or vaccine) is provided in a pre-filled syringe or injector (e.g., single-chambered or multi-chambered). In some embodiments, the composition (e.g., the immunogenic composition or vaccine) is provided for use by mucosal administration (e.g., as an intranasal spray, or sublingually). In some embodiments, the composition (e.g., the immunogenic composition or vaccine) is provided for use by inhalation (e.g., for pulmonary delivery) and is provided in a pre-filled pump, aerosolizer, or inhaler.

In certain embodiments, the compositions of the invention (e.g., the immunogenic compositions or vaccines) are provided for use in skin injection, e.g., in the epidermis, the dermis, or the hypodermis of the skin. In some embodiments, the compositions are provided in a device suitable for skin injection, such as a needle (e.g., an epidermic, dermic or hypodermic needle), a needle free device, a microneedle device, or a microprojection array device. Examples of microneedle or microprojection array devices suitable for the skin injection are described in US20230270842A1, US20220339416A1, US20210085598A1, US20200246450A1, US20220143376A1, US20180264244A1, US20180263641A1, US20110245776A1.

### Therapeutic Uses

In some embodiments, the invention provides a method for eliciting an immune response in a subject, wherein the method comprises administering an effective amount of a composition of the invention (e.g., an immunogenic composition or vaccine of the invention) to a subject. In some embodiments, the invention provides a composition of the invention (e.g., an immunogenic composition or vaccine of the invention) for eliciting an immune response in a subject. In some embodiments, the invention provides for the use of a composition of the invention in a method of manufacturing a medicament, wherein the composition is for (e.g., is formulated for) eliciting an immune response in a subject.

In some embodiments, a composition of the invention (e.g., an immunogenic composition or vaccine of the invention) elicits an immune response against one or more influenza viruses in a subject. In some embodiments, a composition of the invention (e.g., an immunogenic composition or vaccine of the invention) elicits an immune response against influenza A virus. In some embodiments, a composition of the invention (e.g., an immunogenic composition or vaccine of the invention) elicits an immune response against influenza B virus. In some embodiments, a composition of the invention (e.g., an immunogenic composition or vaccine of the invention) elicits an immune response against influenza A and B viruses. In some embodiments, a composition of the invention (e.g., an immunogenic composition or vaccine of the invention) elicits an immune response against influenza viruses of different subtypes and/or lineages.

In some embodiments, a composition of the invention (e.g., an immunogenic composition or vaccine of the invention) is administered prophylactically. In some embodiments, the invention provides a method of reducing the severity of an influenza infection in a subject, the method comprising administering an effective amount of a composition of the invention (e.g., an immunogenic composition or vaccine of the invention) to the subject. In some embodiments, the invention provides a composition of the invention (e.g., an immunogenic composition or vaccine of the invention) for use in reducing the severity of one or more symptoms of an influenza infection in a subject. In some embodiments, the invention provides for the use of a composition of the invention in a method of manufacturing a medicament, wherein the composition is for (e.g., is formulated for) reducing the severity of one or more symptoms of an influenza infection in a subject. In some embodiments, the invention provides a method of reducing the severity of one or more symptoms of an infection with influenza virus A and/or B. In some embodiments, the invention provides a method of reducing the severity of one or more symptoms of infection with influenza viruses of different subtypes and/or lineages.

In some embodiments, the invention provides a method of preventing an influenza infection in a subject, the method comprising administering an effective amount of a composition of the invention (e.g., an immunogenic composition or vaccine of the invention) to the subject. In some embodiments, the invention provides a composition of the invention (e.g., an immunogenic composition or vaccine of the invention) for use in preventing an influenza infection in a subject. In some embodiments, the invention provides for the use of a composition of the invention in a method of manufacturing a medicament, wherein the composition is for (e.g., is formulated for) preventing an influenza infection in a subject. In some embodiments, the invention provides a method of preventing an infection with influenza virus A and/or B. In some embodiments, the invention provides a method of preventing infection with influenza viruses of different subtypes and/or lineages.

In various embodiments, the methods of immunizing provided herein elicit a broadly neutralizing immune response against one or more influenza viruses. In some embodiments, the immune response comprises an antibody response. Accordingly, in various embodiments, the composition described herein can offer broad cross-protection against different types of influenza viruses. In some embodiments, the composition offers cross-protection against avian, swine, seasonal, and/or pandemic influenza viruses.

In some embodiments, the composition offers cross-protection against one or more influenza type A or B and/or one or more subtypes of influenza A or lineages of influenza B, in particular seasonal strains. In some embodiments, the composition offers cross-protection against multiple strains of seasonal influenza viruses. For example, in some embodiments, the composition offers cross-protection against a seasonal influenza A H1-subtype virus (e.g., H1N1), a seasonal influenza A H3-subtype viruses (e.g., H3N2), and one or both circulating influenza B viruses (e.g., influenza B/Yamagata and/or influenza B/Victoria).

In some embodiments, the methods of the invention are capable of eliciting an improved immune response against one or more pandemic influenza strains. Pandemic subtypes include, in particular, the H1N₁, H5N1, H2N2, H3N2, H9N2, H7N7, H7N3, H7N9 and H10N7 subtypes.

In some embodiments, the methods of the invention are capable of eliciting an improved immune response against one or more swine influenza strains.

In some embodiments, the methods of the invention are capable of eliciting an improved immune response against one or more avian influenza strains. Exemplary avian strains include, without limitation, H5N1, H7N3, H7N7, H7N9, and H9N2. Additional influenza pandemic, seasonal, avian and/or swine strains are known in the art.

In some embodiments of the invention, administration of the composition of the invention (e.g., the immunogenic composition or vaccine) provides immunity against an influenza infection caused by a type A strain and/or a type B strain. In some embodiments, administration provides immunity against infection caused by multiple subtypes (e.g., H1N1 and H3N2) or lineages (Victoria and Yamagata) of the same type of influenza (e.g., type A or type B). For example, in some embodiments, immunity is provided against two or more influenza A subtypes. In some embodiments, immunity is provided against one or more (e.g., two) influenza A subtypes (e.g., H1N1 and H3N2) and one or more lineages of influenza type B (e.g., Victoria and/or Yamagata).

In some embodiments, a composition of the invention (e.g., an immunogenic composition or vaccine of the invention) is used as a seasonal and/or pandemic influenza vaccine or as part of an influenza vaccination regimen intended to confer long-lasting (multi-season) protection.

The compositions of the invention (e.g., the immunogenic compositions or vaccines of the invention) may be administered to a subject in need thereof in a therapeutically effective amount, i.e., an amount that provides sufficient immune protection against a target pathogen for a sufficient amount of time (e.g., one year, two years, five years, ten years, or life-time). Sufficient immune protection may be, for example, prevention or alleviation of symptoms associated with infections by the pathogen. In some embodiments, multiple doses (e.g., two doses) of the vaccine are injected to subjects in need thereof to achieve the desired therapeutic effects. The doses (e.g., prime and booster doses) may be separated by an interval of e.g., 1 week, 2 weeks, 3 weeks, 4 weeks, one month, two months, three months, four months, five months, six months, one year, two years, five years, or ten years.

In alternative embodiments, the composition of the invention (e.g., immunogenic composition or vaccine of the invention) is administered following influenza symptoms and/or confirmation that the subject has an influenza infection. In some embodiments, the subject is suffering from or susceptible to an influenza infection. In some embodiments, a subject is considered to be suffering from an influenza infection if the subject is displaying one or more symptoms commonly associated with influenza infection.

In some embodiments, the subject is known or believed to have been exposed to the influenza virus. In some embodiments, a subject is considered to be susceptible to an influenza infection if the subject is known or believed to have been exposed to the influenza virus. In some embodiments, a subject is known or believed to have been exposed to the influenza virus if the subject has been in contact with other individuals known or suspected to have been infected with the influenza virus and/or if the subject is or has been present in a location in which influenza infection is known or thought to be prevalent.

In some embodiments, a composition of the invention (e.g., an immunogenic composition or vaccine of the invention) is administered as a single dose, e.g., by intramuscular administration or mucosal delivery. In some embodiments, a booster dose is administered about one year or more after the first administration. In some embodiments, the booster dose is administered after 5 years.

### Subject

In some embodiments, the subject is a human. In some embodiments, the subject is healthy. In certain embodiments, the subject is an adult, an adolescent, or an infant.

In some embodiments, the subject has an age that puts him or her at a higher risk of developing serious complications from an infection with influenza virus. In some embodiments, the human subject is younger than 6 months old. In some embodiments, the human subject is younger than 2 years old. In some embodiments, the human subject is younger than 5 years old. In some embodiments, the human subject is aged 55 years or older, such as 60 year of age or older, 65 years of age or older, or 70 years of age or older. In some embodiments, the subject is at least 65 years old.

In some embodiments, the subject has a condition that puts him or her at a higher risk of developing serious complications from an infection with influenza virus. In some embodiments, the subject is pregnant. In some embodiments, the subject suffers from pulmonary conditions (e.g., a chronic lung disease). In some embodiments, the subject suffers from asthma. In some embodiments, the subject suffers from COPD.

In some embodiments, the subject lives in a nursing home or another long-term care facility

In some embodiments, the subject is a non-human mammal. In some embodiments, the subject is a farm animal or a pet (e.g., a dog, a cat, a sheep, cattle, and/or a pig). In some embodiments, the subject is a non-human primate. In some embodiments, the subject is an avian (e.g., a chicken).

### EXAMPLES

The following examples are included for illustrative purposes only and are not intended to limit the scope of the invention.

### Example 1: In Vitro Transcription of mRNA

This example illustrates the synthesis of messenger RNA (mRNA) encoding hemagglutinin (HA), neuraminidase (NA) and matrix 1 (M1) protein from influenza A/H1N1 and A/H3N2 viruses.

Sequence-optimized nucleotide sequences encoding the M1 protein from A/California/07/2009, the HA-H1 protein from A/California/07/2009 (H1N1), the NA-N1 protein from A/Michigan/45/2015 (H1N1), the HA-H3 protein from A/Minnesota/07/2008 (H3N2), and the NA-N2 from A/Minnesota/07/2008 (H3N2) were prepared. The codon-optimized sequences were separately inserted in a plasmid comprising suitable 5′ and 3′ UTR sequences and an RNA polymerase promoter. Prior to in vitro transcription (IVT), the plasmids were linearized with a restriction endonuclease.

IVT reactions were performed using linearized DNA template plasmids, NTPs (ATP, GTP, CTP, UTP) and an RNA polymerase in a suitable IVT reaction buffer supplemented with DTT, pyrophosphate and an RNase inhibitor. The IVT reactions were conducted at 37° C. for 90 minutes. The reactions were terminated by treatment with DNase I to remove the template plasmid. The resulting IVT mRNA was purified by precipitation with guanidine thiocyanate.

Purified IVT mRNA was capped by incubation with S-adenosyl methionine, an RNase inhibitor, 2′-O-methyltransferase and a guanylyl transferase in a suitable capping reaction buffer for 90 minutes at 37° C. Following capping, the tailing reaction was performed in a suitable tailing reaction buffer with ATP and a polyA polymerase for 30 minutes at 37° C. The reaction was stopped by addition of EDTA followed by a 5-minute incubation at 37° C. and mRNA purification as described above.

### Example 2: Assessing Neuraminidase (NA) Activity

This example illustrates that NA activity is observed in the supernatant following transfection of mammalian cells with mRNAs encoding influenza virus NA proteins. This activity is also observed if the cells are transfected with more than one mRNA encoding an NA protein, or with mRNAs encoding an M1 protein and one or more HA proteins.

mRNAs encoding HA, NA and M1 proteins were prepared as described in Example 1. HEK293T cells were transfected either with a single mRNA encoding influenza virus proteins N1 or N2, with three mRNAs encoding influenza virus proteins H1, N1 and M1 or H3, N2 and M1, or with five mRNAs encoding influenza virus proteins H1, N1, H3, N2, and M1, respectively. Transfections were performed using the TransIT-mRNA Transfection Kit. Untransfected cells and cells transfected with an eGFP-encoding mRNA were included as negative and positive controls, respectively. The cells were counted, and cell viability was assessed forty-eight hours post-transfection.

The NA activity was determined using supernatants collected from transfected HEK293T. Enzymatic activity was measured using the NA-Fluor™ Influenza Neuraminidase Assay Kit. A standard curve was generated using 4-methylimbelliferone sodium salt and fluorescence was detected using a Varioskan reader following a one-hour incubation at 37° C. Titers of NA activity were measured in μM/h. Three independent determinations were performed for each sample.

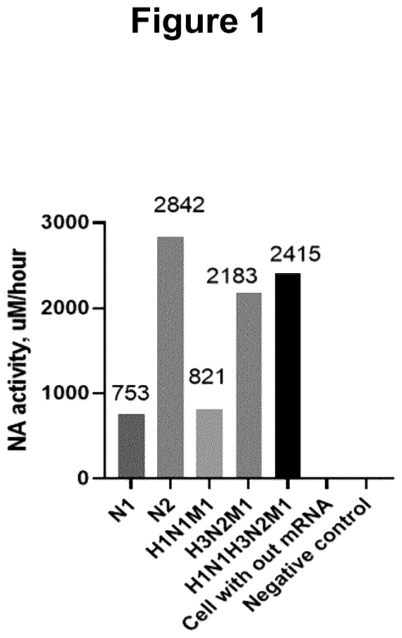

The results are summarized in FIG. 1. NA activity was detected in the supernatant of all cells transfected with an mRNA encoding an NA protein. NA activity was also observed when the cells were additionally transfected with an mRNA encoding an M1 protein, one or two mRNA encoding an NA protein (N1 and/or N2) and one or two mRNA encoding an HA protein (H1 and/or H3). Interestingly, NA activity was about 2.5- to 3-fold higher in cells transfected with an mRNA encoding N2 relative to cells transfected with an mRNA encoding N1. Transfecting cells with both N1 and N2 did not further increase the measured NA activity.

The data in this example indicate that mRNAs encoding the M1, HA and NA proteins can be expressed together in the same cell to produce VLPs with active NA protein.

### Example 3: Assessing Hemagglutinin (HA) Activity

This example illustrates that mRNAs encoding influenza virus HA proteins are capable of inducing mammalian cells to express functional hemagglutinin (HA) activity.

mRNAs encoding HA, NA and M1 proteins were prepared as described in Example 1. HEK293T cells were transfected with a single mRNA encoding either H1 or H3, with two mRNAs encoding either the H1 or H3 protein and the M1 protein, with three mRNAs encoding either the H1, N1 and M1 proteins, the H3, N2 and M1 proteins or the H1, H3 and M1 protein, or with five mRNAs encoding influenza proteins H1, N1, H3, N2 and M1, respectively. The transfection was performed in the same manner as described in Example 2.

The functionality of HA was determined by calculating a hemagglutinin unit (HAU) titer using supernatants from HEK293T cells transfected with the mRNAs. Serial dilutions of said supernatants were incubated with 50 μL of 0.5% (v/v) turkey red blood cells for 45-60 minutes at 4° C. The HAU titer of a suspension is defined as being the reciprocal of the highest dilution that shows complete hemagglutination. Hemagglutination values for both non-concentrated and concentrated (5×) supernatants are shown in Table 1. Previously purified and concentrated VLPs prepared with the H1 and N1 proteins of influenza virus A/Michigan/45/2015 and the H3 and N2 proteins of A/Hong Kong/4801/2014, respectively, served as positive controls. The supernatant from cells transfected without mRNA were used as a negative control.

TABLE 1
Hemagglutination values of supernatants from mRNA-transfected cells

| | HAU titers/50 μL from supernatant | | | | | | | | | |
|---|---|---|---|---|---|---|---|---|---|---|
| | | | | | | | | H1 | | |
| | | | | | | | | N1 | | |
| | | | | | H1 | H3 | H1 | H3 | | |
| | | | H1 | H3 | N1 | N2 | H3 | N2 | | |
| Samples | H1 | H3 | M1 | M1 | M1 | M1 | M1 | M1 | PC | NC |
| Non- | Neg | Neg | Neg | Neg | 1 | Neg | Neg | 1 | 1024 | Neg |
| concentrated | | | | | | | | | | |
| Concentrated | 8 | Neg | 2 | Neg | 8 | Neg | 4 | 2 | 2048 | Neg |

*Neg: negative;
PC: positive control;
NC: negative control

Hemagglutination was observed for all concentrated supernatants obtained from cells transfecting with an mRNA encoding the H1-subtype HA protein, indicating that cells expressed the protein in its active form. This observation was independent of the cells being additionally transfected with one or more mRNAs encoding another HA protein (H3), one or more NA proteins (N1, or N1 and N2) and/or with an mRNA encoding an M1 protein.

In the absence of the H1 subtype HA protein, no hemagglutination was observed for cells transfected with an mRNA-encoding the H3 subtype HA protein. This was not entirely surprising as hemagglutination can be difficult to detect with H3-subtype HA proteins. To confirm that the H3-subtype HA protein was expressed, a Western blot was performed with a polyclonal anti-H3 antibody (rabbit, SIGMA). Briefly, cell lysates were separated on a NuPAGE gel (Invitrogen). The separated proteins were blotted to a nitrocellulose membrane (BioRAD) and probed with the anti-H3 antibody. An anti-rabbit antibody (Rockland, DyLight) was used for detection using an Odyssey imaging system (LICOR).

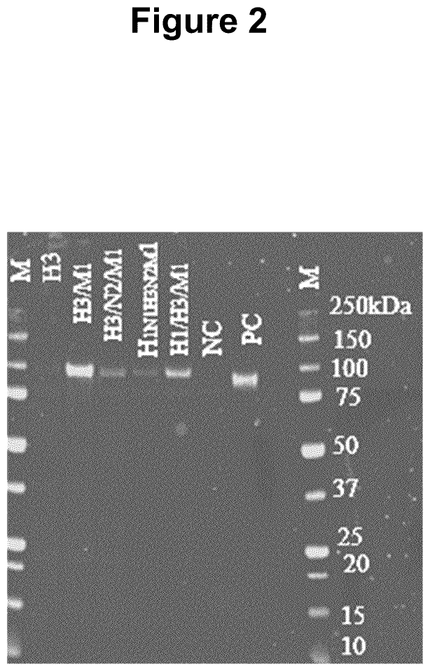

As can be seen from FIG. 2, H3 protein was readily detected in cells transfected with mRNAs encoding (i) the H3 and M1 proteins, (ii) the H3, N2 and M1 proteins, (iii) the H1, N1, H3, N2 and M1 proteins, and (iv) the H1 and H3 proteins. For unknown reason, only a very faint band of H3 was detected when cells were transfected with a single mRNA encoding the H3 protein.

The data in this example indicate that mRNAs encoding the M1, HA and NA proteins can be expressed together in the same cell to produce VLPs with functional HA protein.

### Example 4: Visualization of Influenza VLPs by Negative Staining Transmission Electron Microscopy (NS-TEM)

This example illustrates that mammalian cells transfected with mRNAs encoding M1, HA and NA proteins are capable of producing VLPs. This example also illustrates that the budding of VLPs is greatly reduced when mammalian cells are transfected only with mRNAs encoding M1 and HA proteins.

In order to visualize the generation of VLPs in mammalian cells transfected with mRNAs encoding the M1, HA and NA proteins, negative staining transmission electron microscopy (NS-TEM) was performed. HEK293T cells were transfected with three different combinations of mRNAs. mRNAs encoding HA, NA and M1 proteins were prepared as described in Example 1. Some cells were transfected with a combination of three mRNAs encoding the M1 protein and either two HA proteins (H1 and H3), or one HA protein (H1 or H3, respectively) and one NA protein (N1 or N2, respectively). Other cells were transfected with five mRNAs encoding the M1 protein, two HA protein (H1 and H3) and two NA proteins (N1 and N2).

Cell supernatants of the transfected cells were incubated on a previously ionized continuous carbon film TEM grids in a plasma cleaner. Excess material was absorbed using filter paper, and adsorbed particles were negatively stained with 2% uranyl acetate and imaged.

The supernatants of HEK293T cells transfected with mRNAs encoding (i) H1, N1, H3, N2 and M1, (ii) H1, N1 and M1, (iii) H3, N2, and M1, and (iv) H1, H3, and M1 contained vesicles, indicating the production and release of VLPs by the transfected cells. No vesicles were observed in the supernatant of mock transfected cells. Importantly, only very low amounts of vesicles were observed in the supernatant of cells transfected with mRNAs encoding H1, H3, and M1.

This example illustrates that mammalian cells transfected with mRNAs encoding M1, HA and NA proteins are capable of producing VLPs. This example also illustrates that VLP formation can still occur when mammalian cells are transfected with mRNAs encoding M1 and HA proteins only. However, VLP formation is greatly diminished, indicating that efficient budding of VLPs from the cell surface is linked to the expression of the NA protein.

### Example 5: Visualization of Influenza VLPs by Cryogenic Transmission Electron Microscopy (Cryo-TEM)

This example illustrates that mammalian cells transfected with an mRNA encoding a M1 protein, and mRNA encoding more than one HA protein and more than one NA protein are capable of producing VLPs, even when the coding sequences are derived from different influenza viruses.

In order to determine the size of VLPs produced by mRNA-transfected mammalian cells, cryogenic transmission electron microscopy (Cryo-TEM) was performed to observe the budding of the VLPs from the cell membrane. mRNAs encoding HA, NA and M1 proteins were prepared as described in Example 1. HEK293T cells were transfected either with three mRNAs encoding the M1 protein, one HA protein (H3) and one NA protein (N2), or with five mRNAs encoding the M1 protein, two HA proteins (H1 and H3) and two NA proteins (N1 and N2). Mock-transfected cells (no mRNA) were included as control. Samples were deposited on Quantifoil R2/2 copper 200 mesh grids following glow discharge on an ELMO ionizer. Grids were blotted, frozen and transferred for visualization by Cryo-TEM.

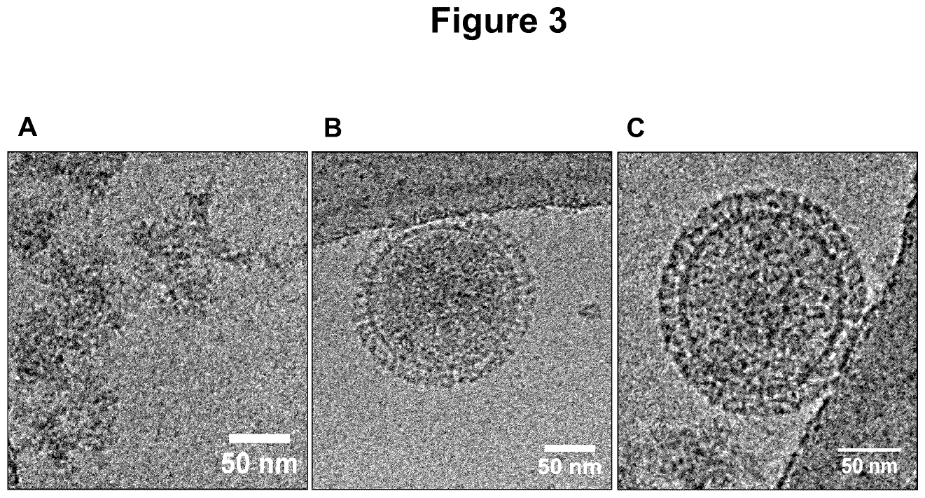

Illustrative images are shown in FIG. 3. Panel A shows mock-transfected cells. No VLPs were visible. Panels B and C show representative images of VLPs from cells transfected either with a set of three mRNAs (H3/N2/M1; panel B) or five mRNAs (H1/N1/H3/N2/M1; panel C). VLPs could be observed with heterogenous spikes of glycoproteins resembling the glycoproteins of influenza viruses. The observed VLPs were approximately 100 nm in size and therefore similar to the size of influenza viruses. Notably, this was the case even when the cells were transfected with mRNA encoding HA proteins and NA proteins from different influenza viruses (A/California/07/2009 [H1], and A/Michigan/45/2015 [N1] and A/Minnesota/07/2008 [H3 and N2], respectively), or when the M1 protein and the HA and NA proteins were from different influenza viruses (A/California/07/2009 [H1N1] and A/Minnesota/07/2008 [H3N2], respectively).

This example demonstrates that mammalian cells transfected with an mRNA encoding an M1 protein and mRNAs encoding two HA proteins and two NA proteins from different influenza viruses are capable of producing VLPs. Similarly, the coding sequence of the mRNA encoding the M1 protein can be derived from a different virus than the coding sequences of the mRNAs encoding the HA and NA proteins

### Example 6: Transfection with Influenza A or B Proteins Enables VLP Formation

This example illustrates that mRNAs encoding both M1 and influenza glycoproteins derived from either type A or B are capable of producing VLPs.

In order to determine whether VLPs from both influenza virus types A and B could be produced, four different strains were investigated. Sequence-optimized nucleotide sequences encoding the HA and NA proteins (H3N2) from A/Darwin/6/2021, the HA and NA proteins (H1N1) from A/Wisconsin/588/2019, the HA and NA proteins from B/Phuket/3073/2013 (B/Yamagata lineage), and the HA and NA proteins from B/Austria/1359417/2021 (B/Victoria lineage) were prepared. As in the previous examples, the same sequence-optimized nucleotide sequence encoding the M1 protein from A/California/07/2009 was used. The codon-optimized sequences were separately inserted in a plasmid comprising suitable 5′ and 3′ UTR sequences and an RNA polymerase promoter. Prior to IVT, the plasmids were linearized with a restriction endonuclease. IVT reactions were performed as described in Example 1.

Expi293F cells were transfected with three mRNAs encoding influenza virus proteins using the method described in Example 2. Four combinations (Darwin, Wisconsin, Phuket and Austria) were tested, as shown in Table 2. For each transfection, mRNAs encoding HA, NA, and M1 were transfected at a 40:10:50 molar ratio, respectively.

TABLE 2
Tested mRNA combinations

| | | | |
|---|---|---|---|
| | | | Short |
| HA mRNA | NA mRNA | M1 mRNA | name |

| | | |
|---|---|---|
| type/strain designation (subtype/lineage) | type/strain | |
| | designation | |
| A/Darwin/6/2021 (H3N2) | A/California/ | Darwin |
| A/Wisconsin/588/2019 (H1N1) | 07/2009 | Wisconsin |
| B/Phuket/3073/2013 (Yamagata) | | Phuket |
| B/Austria/1359417/2021 (Victoria) | | Austria |

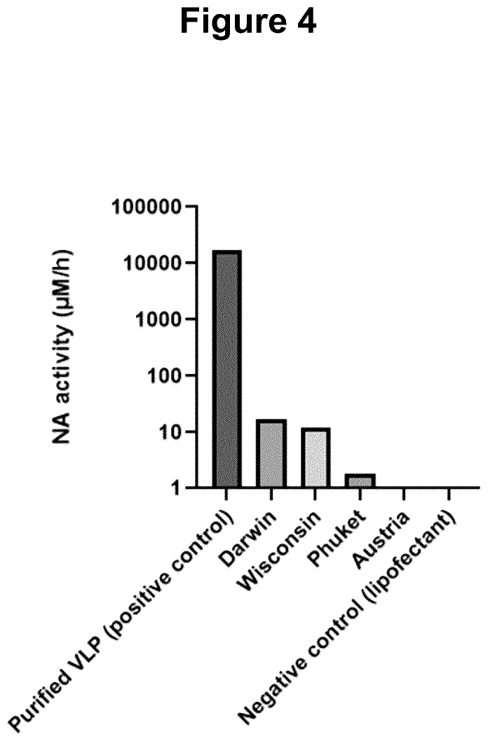

Enzymatic NA activity assays using supernatants collected from transfected Expi293F cells were performed by the method as described in Example 2. The results are summarized in FIG. 4. NA activity was detectable in all supernatants collected from transfected Expi293F cells, apart from the Austria strain. Relative to purified VLPs (positive control), NA activity was low in this experiment. Western blot confirmed weak NA protein expression (data not shown).

M1 and HA expression in cell lysates and supernatant extracts from the transfected Expi293F cells was assessed by Western blot. The reagents used for detection are shown in Table 3.

TABLE 3
Reagents used for detection of HA and M1

| Antibody | Description | Supplier | Reference | Dilution |
|---|---|---|---|---|
| Primary | Influenza A virus | Sinobiological | 86001- | 1/2000 |
| | anti-HA Ab, rabbit | | RM01 | |
| | Mab | | | |
| | Influenza B virus | | 11053- | |
| | anti-HA Ab, rabbit | | R004 | |
| | Mab | | | |
| | Influenza A M1 | Invitrogen | PA5- | 1/5000 |
| | polyclonal serum | | 32222 | |
| Secondary | Anti-Rabbit IgG | Rockland | 611-145- | 1/5000 |
| | Dylight 800 | | 002 | |
| | conjugated | | | |

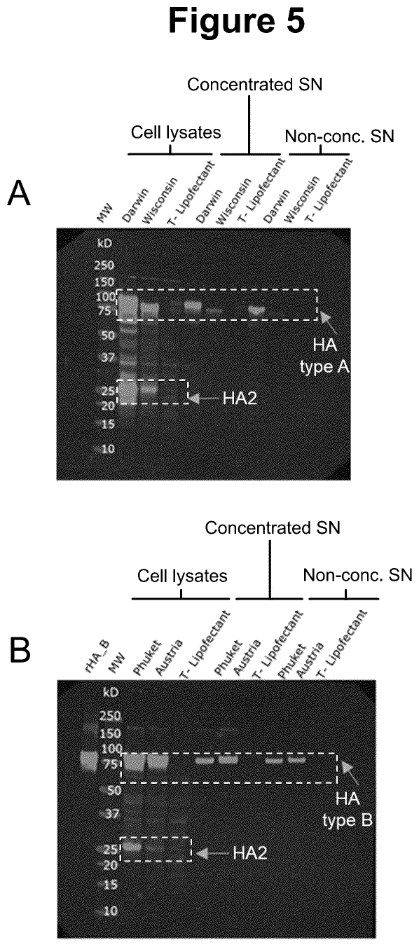

Both concentrated (25×) and non-concentrated supernatant extracts were analyzed. HA expression was determined using a monoclonal influenza A or B strain anti-HA antibody. The results are shown in FIGS. 5A (influenza type A) and FIG. 5B (influenza type B), respectively. HA expression was detected in the cell lysates, concentrated supernatant extracts and non-concentrated supernatant extracts of all experimental conditions. No signal was detected in lysates and supernatant of mock-transfected cells (negative control). HA is a homotrimer in which each protomer consists of an HA1 and an HA2 chain connected through a single disulfide bridge. The HA2 subunit could be detected in each of the cell lysates, but not the cell supernatants.

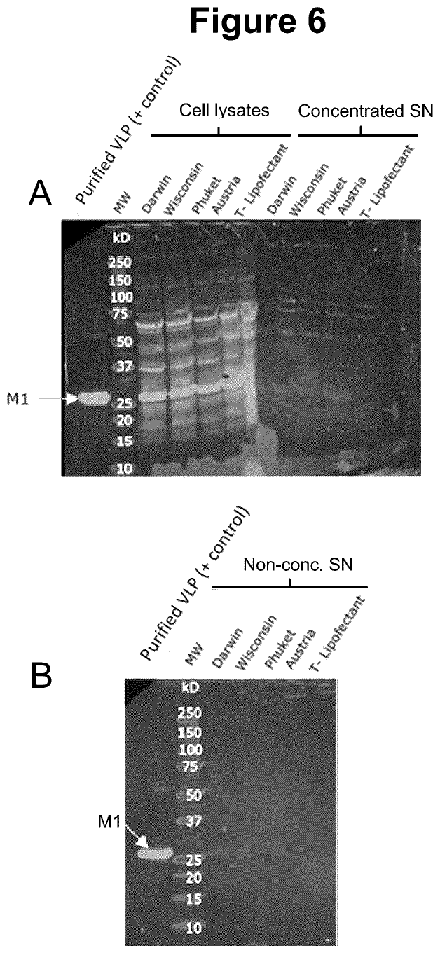

M1 expression was determined using a polyclonal anti-M1 antibody. As shown in FIG. 6, M1 was detected in the cell lysates and concentrated supernatant of transfected cells (panel A) but not the non-concentrated supernatant samples (panel B). Only trace amounts could be detected in the concentrated supernatants. Band intensity was greatest in the cell lysate samples. Non-specific binding was observed in the samples comprising cell lysate and concentrated supernatants, including the negative control samples. However, the M1-specific band was clearly distinguishable. No M1 was observed in cell lysates and supernatants of mock-transfected cells (lipofectant only, negative control).

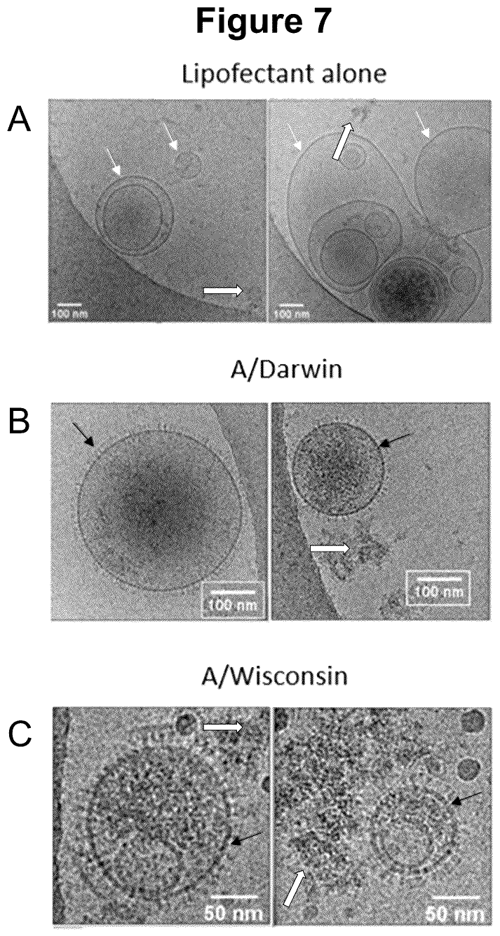

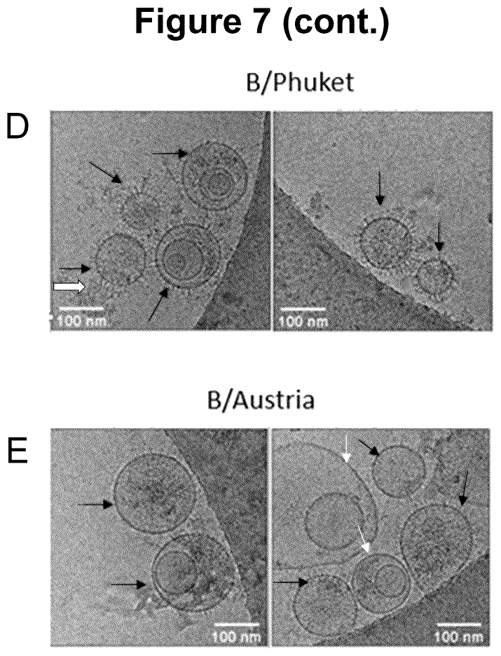

VLPs produced by mRNA-transfected mammalian cells were visualized using Cryo-TEM, as described in Example 5. Illustrative images are shown in FIG. 7. Panel A shows representative images of mock-transfected cells. No VLPs were found in this condition. Only heterogeneous smooth vesicles (white arrows), likely artefacts from the transfection process, and protein debris (black outlined arrows) were observed.

Representative images of VLPs from cells transfected with M1 plus HA and NA from the influenza A strains Darwin and Wisconsin are shown in panels B and C, respectively. Panels D and E show representative images of VLPs from cells transfected with M1 plus HA and NA from the influenza B strains Phuket and Austria, respectively. VLPs densely decorated with glycoproteins (black filled arrows) were observed in all four conditions. The size of the VLPs ranged from around 50-300 nm, with the majority being above 100 nm in size.

This example demonstrates that mammalian cells transfected with an mRNA encoding an M1 protein and mRNAs encoding HA and NA from either influenza type A or type B are capable of producing VLPs. The data provide further support for the use of the same coding sequence of the M1 protein regardless of the influenza type and strain from which the HA and NA proteins derive.

### Example 7: Mouse Immunization

This example outlines a mouse study to test the immunogenicity of compositions comprising mRNAs encoding the HA, NA and M1 proteins described in Example 6.

A monovalent mRNA composition comprising three sequence-optimized mRNAs encoding the HA and NA proteins of influenza strain B/Phuket/3073/2013 (Yamagata) and the M1 protein of A/California/07/2009 is prepared. The mRNAs are encapsulated in a lipid nanoparticle (LNP) comprising the cationic lipid GL-HEPES-E3-E12-DS-4-E10, the non-cationic lipid DOPE, cholesterol, and the PEG-modified lipid DMG-PEG-2K at molar ratios of 40:30:28.5:1.5. The final mRNA-LNP formulations are provided in an aqueous suspension. The mRNAs encoding HA, NA, and M1 are provided at a weight ratio 1:1:2, respectively, in the final composition used for immunization.

In parallel, a quadrivalent mRNA composition comprising four sets of mRNAs is prepared. Set 1 comprises three sequence-optimized mRNAs encoding the HA and NA proteins of influenza strain B/Phuket/3073/2013 (Yamagata) and the M1 protein of A/California/07/2009. Set 2 comprises three sequence-optimized mRNAs encoding the HA and NA proteins of influenza strain A/Darwin/6/2021 and A/Tasmania/503/2020 (H3N2), respectively, and the M1 protein of A/California/07/2009. Set 3 comprises three sequence-optimized mRNAs encoding the HA and NA proteins of influenza strain A/Wisconsin/588/2019 (H1N1) and the M1 protein of A/California/07/2009. Set 4 comprises three sequence-optimized mRNAs encoding the HA and NA proteins of influenza strain B/Austria/1359417/2021 and B/Washington/02/2019 (Victoria), respectively, and the M1 protein of A/California/07/2009. Each of the sets of mRNAs is separately encapsulated in the same LNP as the monovalent composition. For each set, the mRNAs encoding HA, NA, and M1 are provided at a weight ratio 1:1:2, respectively, in the final composition used for immunization.

As controls, mRNA compositions corresponding to the monovalent and quadrivalent compositions are provided in which the mRNA encoding the M1 protein is replaced with an mRNA encoding a T epitope from Epstein Barr virus (EBV). The quadrivalent recombinant influenza vaccine Flublok and the quadrivalent inactivated split virion vaccine Vaxigrip Tetra are provided as immunization controls. The HA and NA proteins in Flublok and Vaxigrip Tetra match those encoded by the respective HA and NA mRNAs in the quadrivalent mRNA composition. Phosphate-buffered saline (PBS) serves as a negative control.

Each of the compositions is administered to mice via intramuscular (IM) injection at day 0 and day 21. Nine groups of BALB/c mice are immunized (n=8). Group 1 receives 2 μg mRNA total per injection of the monovalent mRNA composition. Group 2 receives 8 μg mRNA total per injection of the monovalent mRNA composition. Groups 3 and 4 receive the monovalent control mRNA composition at 2 μg and 8 μg mRNA total per injection, respectively. Group 5 receives 8 μg mRNA total per injection of the quadrivalent mRNA composition. Group 6 receives 8 μg mRNA total per injection of the control quadrivalent mRNA composition. Group 7 receives Flublok at 1/10th of the human dose per injection. Group 8 receives Vaxigrip Tetra at 1/10^{th} of the human dose per injection. Group 9 receives PBS.

On day 42, the mice are sacrificed. Blood is collected to assess IgG production (total IgG) and anti-HA/anti-NA functional antibodies using ELISA, Hemagglutination Inhibition (HAI) assay, and neuraminidase activity inhibition (NAI) assay, respectively.

1. A composition comprising one or more messenger RNAs (mRNAs) encoding (i) an influenza virus matrix 1 (M1) protein and (ii) one or more influenza virus hemagglutinin (HA) proteins and/or one or more influenza virus neuraminidase (NA) proteins, wherein the M1 protein and the one or more HA proteins and/or the one or more NA proteins are capable of inducing a mammalian cell to produce a virus-like particle (VLP).

2. The composition of claim 1, wherein the one or more mRNAs encode two or more HA proteins and/or two or more NA proteins, wherein each of the two or more HA proteins and each of the two or more NA proteins are from different influenza viruses.

3. The composition of claim 2, wherein at least one of the two or more HA proteins and/or at least one of the two or more NA proteins is/are from an influenza virus that is different from the influenza virus from which the M1 protein originates.

4. The composition of claim 3, wherein the two or more HA proteins and/or the two or more NA proteins are from different influenza A subtypes, and/or at least one of the two or more HA proteins and/or at least one of the two or more NA proteins is/are from an influenza B virus.

5. (canceled)

6. The composition of claim 1, wherein the M1 protein is from a pandemic influenza virus or from a H1N1 influenza A virus.

7. (canceled)

8. The composition of claim 1, wherein the M1 protein has a serine at position 30, an alanine at position 142, an asparagine at position 207, and a threonine at position 209.

9. The composition of claim 1, wherein the M1 protein is encoded by a separate mRNA.

10. The composition of claim 2, wherein at least one of the two or more NA proteins has an activity of 2000 μM/h or greater, as determined by a neuraminidase activity assay.

11. The composition of claim 4, wherein the two or more NA proteins comprise two NA proteins from different influenza A subtypes.

12. The composition of claim 11, wherein the different influenza A subtypes are N1 and N2.

13. The composition of claim 11, wherein the composition is capable of inducing expression of a VLP in the mammalian cell, wherein the VLP comprises the two NA proteins from different influenza A subtypes.

14. The composition of claim 4, wherein the two or more HA proteins comprise two HA proteins from different influenza A subtypes.

15. The composition of claim 14, wherein the different influenza A subtypes are H1 and H3.

16-17. (canceled)

18. The composition of claim 1, wherein each HA protein and each NA protein are encoded by separate mRNAs.

19-21. (canceled)

22. The composition of claim 1, wherein the composition comprises a first mRNA encoding a first HA protein and a first NA protein, a second mRNA encoding a second HA protein and a second NA protein, and a third mRNA encoding the M1 protein, wherein the first HA protein and the first NA protein are from a first influenza A virus, and wherein the second HA protein and the second NA protein are from a second influenza A virus.

23-24. (canceled)

25. The composition of claim 22, wherein the composition further comprises a fourth mRNA encoding a third HA protein and a third NA protein, wherein the third HA protein and the third NA protein are from a third influenza virus.

26. The composition of claim 25, wherein the third influenza virus is an influenza B virus.

27-28. (canceled)

29. The composition of claim 22, wherein the first, second, fourth and fifth mRNA, as applicable, comprise in 5′ to 3′ order (i) the coding sequence of the HA protein, (ii) a nucleotide sequence encoding an internal ribosome entry site (TRES) or a 2A peptide, and (iii) the coding sequence of the NA protein.

30-49. (canceled)

50. A method of treating or preventing an influenza infection in a subject comprising administering one or more messenger RNAs (mRNAs) encoding (i) an influenza virus matrix 1 (M1) protein and (ii) one or more influenza virus hemagglutinin (HA) proteins and/or one or more influenza virus neuraminidase (NA) proteins, wherein the M1 protein and the one or more HA proteins and/or the one or more NA proteins are capable of inducing a mammalian cell to produce a virus-like particle (VLP).

51-56. (canceled)

57. A method of eliciting an immune response against one or more influenza viruses in a subject, wherein the method comprises administering to the subject in need thereof a pharmaceutical composition or vaccine comprising one or more messenger RNAs (mRNAs) encoding (i) an influenza virus matrix 1 (M1) protein and (ii) one or more influenza virus hemagglutinin (HA) proteins and/or one or more influenza virus neuraminidase (NA) proteins, wherein the M1 protein and the one or more HA proteins and/or the one or more NA proteins are capable of inducing a mammalian cell to produce a virus-like particle (VLP).

58-63. (canceled)
